SEQUENCE LISTING (13 sequences)

SEQ ID NO: 1 - DNA, 28 bp, Synthetic construct
  source 1..28: mol_type = other DNA
aatgatacggcgaccaccgagactacac

SEQ ID NO: 2 - DNA, 23 bp, Synthetic construct
  source 1..23: mol_type = other DNA
caagcagaagacggcatacgaat

SEQ ID NO: 3 - DNA, 23 bp, synthetic construct
  source 1..23: mol_type = other DNA
caagcagaagacggcatacagat

SEQ ID NO: 4 - DNA, 35 bp, Synthetic construct
  source 1..35: mol_type = other DNA
gctggcacgtccgaacgcttcgttaatccgttgag

SEQ ID NO: 5 - DNA, 35 bp, Synthetic construct
  source 1..35: mol_type = other DNA
cgtcgtctgccatggcgcttcggtggatatgaact

SEQ ID NO: 6 - DNA, 35 bp, Synthetic construct
  source 1..35: mol_type = other DNA
acggccgctaatatcaacgcgtcgaatccgcaact

SEQ ID NO: 7 - DNA, 34 bp, Synthetic construct
  source 1..34: mol_type = other DNA
gccgcgttacgttagccggactattcgatgcagc

SEQ ID NO: 8 - DNA, 29 bp, Synthetic construct
  source 1..29: mol_type = other DNA
  modified_base 23: mod_base = OTHER; note = uracil
aatgatacggcgaccaccgagatctacac

SEQ ID NO: 9 - DNA, 29 bp, Synthetic construct
  source 1..29: mol_type = other DNA
  modified_base 23: mod_base = OTHER; note = Allyl-T
aatgatacggcgaccaccgaganctacac

SEQ ID NO: 10 - DNA, 24 bp, Synthetic construct
  source 1..24: mol_type = other DNA
  modified_base 22: mod_base = OTHER; note = 8-oxoguanine
caagcagaagacggcatacganat

SEQ ID NO: 11 - DNA, 24 bp, synthetic construct
  source 1..24: mol_type = other DNA
  modified_base 20: mod_base = OTHER; note = 8-oxoguanine
caagcagaagacggcatacnagat

SEQ ID NO: 12 - DNA, 24 bp, Synthetic construct
  source 1..24: mol_type = other DNA
  modified_base 22: mod_base = OTHER; note = 8-oxoguanine
  modified_base 20: mod_base = OTHER; note = 8-oxoguanine
caagcagaagacggcatacnanat

SEQ ID NO: 13 - DNA, 24 bp, Synthetic construct
  source 1..24: mol_type = other DNA
aggaggaggaggaggaggaggagg

(19) United States
(12) Patent Application Publication
(10) Pub. No.: US 2024/0426739 A1
(43) Pub. Date: Dec. 26, 2024

(54) FLOW CELLS AND METHODS FOR PREPARING SURFACES THEREOF

(71) Applicant: ILLUMINA, INC., San Diego, CA (US)

(72) Inventors: Jeffrey S. Fisher, San Diego, CA (US); Justin Fullerton, San Diego, CA (US); Brian D. Mather, San Diego, CA (US); Maxwell Zimmerley, San Diego, CA (US)

(21) Appl. No.: 18/739,578

(22) Filed: Jun. 11, 2024

Related U.S. Application Data
(60) Provisional application No. 63/507,941, filed on Jun. 13, 2023.

(51) Int. Cl. G01N 15/1434

(52) U.S. Cl. CPC G01N 15/1436

(57) ABSTRACT

An example flow cell includes a first substrate; a second substrate attached to the first substrate; a flow channel defined between the first substrate and the second substrate; a first primer set attached to the first substrate, the first primer set including an un-cleavable first primer and a cleavable second primer; a second primer set attached to the second substrate, the second primer set including a cleavable first primer and an un-cleavable second primer; and a removable blocking mechanism passivating i) the first primer set or the second primer set or ii) the cleavable first primer and the cleavable second primer.

### CROSS-REFERENCE TO RELATED APPLICATIONS

This application claims the benefit of U.S. Provisional Application Ser. No. 63/507,941, filed Jun. 13, 2023, the content of which is incorporated by reference herein in its entirety.

### REFERENCE TO SEQUENCE LISTING

The Sequence Listing submitted herewith is hereby incorporated by reference in its entirety. The name of the file is ILI260B_IP-2564-US_Sequence_Listing.xml, the size of the file is 15,772 bytes, and the date of creation of the file is Jun. 5, 2024.

### BACKGROUND

Some available platforms for sequencing nucleic acids utilize a sequencing-by-synthesis approach. With this approach, a nascent strand is synthesized, and the addition of each monomer (e.g., nucleotide) to the growing strand is detected optically and/or electronically. Because a template strand directs synthesis of the nascent strand, one can infer the sequence of the template DNA from the series of nucleotide monomers that were added to the growing strand during the synthesis. In some examples, sequential paired-end sequencing may be used, where forward strands are sequenced and removed, and then reverse strands are constructed and sequenced. In other examples, simultaneous paired-end sequencing may be used, where forward strands and reverse strands are sequenced at the same time.

### SUMMARY

Some of the methods disclosed herein prepare flow cell surfaces for simultaneous paired-end sequencing. With some examples of simultaneous paired-end sequencing, two different primer sets are attached to different regions of a flow cell, two different lanes of a flow cell, or two different flow cells. Together these two primer sets enable forward strands and reverse strands of a seeded library template to be respectively generated in the different regions. With other examples of simultaneous paired-end sequencing, the same primer set, where the primers have opposite linearization chemistries, is attached to different lanes of a flow cell or two different flow cells.

In some example methods, primers of one of the two primer sets are blocked, via a blocking mechanism, while primers of the other of the two primer sets are seeded with a library template, and the library template is amplified. Subsequent to seeding and amplification, the blocking mechanism is removed, and processes are performed to seed and amplify a complement of the library template using the unblocked primers of the other of the two primer sets.

In some other example methods, cleavable primers of the two different primer sets are blocked while un-cleavable primers of the different primer sets are simultaneously seeded. Subsequent to seeding, the blocking mechanism is removed and the seeded library templates are amplified.

The blocking mechanism in these example methods temporarily passivates one primer set or the cleavable primers of both of the primer sets, and leaves, respectively, the other primer set or the un-cleavable primers active for seeding.

In still other example methods, one of the two different primer sets is incorporated into the initial flow cell. A library template is seeded and amplified using this primer set. Subsequent to seeding and amplification, the other of the two different primer sets is grafted to another region of the flow cell and is used in seeding and amplification.

With the blocking mechanisms disclosed herein or the timing of the introduction of the second of the two different primer sets into the flow cell, intricate and complex nanofabrication techniques often used to generate adjacent regions for the two different primer sets are avoided.

With any of the methods disclosed herein, unique molecular indices (UMI) or unique dual indices (UDI) may be incorporated into the library template(s) that are seeded and amplified. These indices aid in linking two reads (e.g., from a library template and its complement) bioinformatically. As an example, the forward and reverse strands of a library template can be linked via the unique molecular indexes, along with the proximity of the strands to one another within a reference genome. As another example, several library templates strands and complements containing the same UDIs may be grouped together.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A is a semi-schematic top view of an example of a flow cell;

FIG. 1B is a cross-sectional view of one example of the architecture within a lane of the flow cell including opposed patterned substrates, where a primer set on one of the patterned substrates is blocked;

FIG. 1C is a cross-sectional view of another example of the architecture within a lane of the flow cell including opposed patterned substrates, where cleavable primers of both of the patterned substrates are blocked;

FIG. 1D is a cross-sectional view of still another example of the architecture within a lane of the flow cell including one patterned substrate and a lid, wherein one primer set in one region of the patterned substrate is blocked;

FIG. 1E is a cross-sectional view of yet another example of the architecture within a lane of the flow cell including one patterned substrate and a lid, wherein the cleavable primers in both regions of the patterned substrate are blocked;

FIG. 1F is a cross-sectional view of yet a further example of the architecture within a lane of the flow cell including two patterned substrates, one of which includes capture primers and the other of which includes blocked capture primers (not shown);

FIG. 2A is a schematic view of two primer sets that are suitable for simultaneous paired-end sequencing;

FIG. 2B is a schematic view of two other primer sets that are suitable for simultaneous paired-end sequencing;

FIG. 3A through FIG. 3D illustrate various removable blocking mechanisms for a single primer set, where FIG. 3A depicts linearizable blocking primers, FIG. 3B depicts blocking primers, FIG. 3C depicts linearizable hairpin primers, and FIG. 3D depicts a hydrophobic barrier layer;

FIG. 4A and FIG. 4B illustrate various removable blocking mechanisms for cleavable primers, where FIG. 4A depicts terminal phosphate groups, and FIG. 4B depicts linearizable hairpin primers;

FIG. 5A through FIG. 5F are schematic illustrations that together illustrate an example method involving the flow cell of FIG. 1B or FIG. 1D, where FIG. 5A depicts a library template seeded to one of the primers of a first primer set while the second primer set is blocked, FIG. 5B depicts first and second amplicons generated from amplification of the seeded library template, FIG. 5C depicts the generation of first amplicon complements and the removal of the second amplicons, FIG. 5D depicts the removal of the blocking mechanism from the second primer set, FIG. 5E depicts dehybridized first amplicon complements seeded to one of the primers of the second primer set, and FIG. 5F depicts the amplicons generated from the first amplicon complements using the second primer set;

FIG. 6A through FIG. 6D are schematic illustrations that together illustrate another example method involving the flow cell of FIG. 1C or FIG. 1E, where FIG. 6A depicts respective library templates seeded to the un-cleavable primers of each of the primer sets while the cleavable primers of each primer set are blocked, FIG. 6B depicts first and second amplicons generated from amplification of the seeded library templates, FIG. 6C depicts the removal of the seeded library templates and the blocking mechanism, and FIG. 6D depicts amplicons generated after simultaneous amplification of the first and second amplicons;

FIG. 7 is a cross-sectional view of still another example of the architecture within a lane of the flow cell including opposed patterned substrates, where one of the substrates does not yet have a primer set introduced thereto;

FIG. 8A through FIG. 8F are schematic illustrations that together illustrate an example method involving the flow cell of FIG. 7, where FIG. 8A depicts a library template seeded to the un-cleavable primer of the first primer set, FIG. 8B depicts the grafting of the second primer set to the opposed substrate, FIG. 8C depicts the generation of first and second amplicons using the first primer set, FIG. 8D depicts cleavage of second amplicons from the cleavable primer of the first primer set and the generation of a first amplicon complement, FIG. 8E depicts dehybridized first amplicon complements seeded to one of the primers of the second primer set, and FIG. 8F depicts the amplicons generated from the first amplicon complements using the second primer set;

FIG. 9 is a cross-sectional view of an example architecture within two lanes of a flow cell including a patterned substrate and a lid, where each lane includes the same primer set;

FIG. 10A through FIG. 10F are schematic illustrations that together illustrate an example method involving the flow cell of FIG. 9, where FIG. 10A depicts a library template seeded to one of the primers in a first lane or flow cell, FIG. 10B depicts first and second amplicons generated from amplification of the seeded library template, FIG. 10C depicts the generation of first amplicon complements and the removal of the second amplicons, FIG. 10D depicts dehybridized first amplicon complements seeded to one of the primers in the second lane or flow cell, FIG. 10E depicts third and fourth amplicons generated from the first amplicon complements using the primers in the second lane or flow cell, and FIG. 10F depicts the first and fourth amplicons in the respective lanes or flow cells; and

FIG. 11 is an enlarged, cross-sectional view depicting another example of the flow cell architecture integrated into a complementary metal oxide semiconductor (CMOS) device.

### DETAILED DESCRIPTION

Some of the methods disclosed herein generate flow cells that are suitable for use in simultaneous paired-end sequencing. For simultaneous paired-end sequencing, different primer sets are attached to different regions of the flow cell. The methods disclosed herein enable library templates to be seeded and clustered in one region, and the complements of the library templates to the seeded and clustered in another region. The primer sets may be controlled so that the cleaving (linearization) chemistry is orthogonal in the different regions. Orthogonal cleaving chemistries are susceptible to different cleaving agent(s). This enables a cluster of forward strands to be generated in one region and a cluster of reverse strands to be generated in another region. The forward and reverse strands are spatially separate, which separates the fluorescence signals from both reads while allowing for simultaneous base calling of each read. Unique molecular indices or unique dual indices incorporated into the library template(s) aid in linking two reads bioinformatically.

### Definitions

It is to be understood that terms used herein will take on their ordinary meaning in the relevant art unless specified otherwise. Several terms used herein and their meanings are set forth below.

The singular forms “a”, “an”, and “the” include plural referents unless the context clearly dictates otherwise.

The terms comprising, including, containing and various forms of these terms are synonymous with each other and are meant to be equally broad.

The terms top, bottom, lower, upper, on, etc. are used herein to describe the flow cell and/or the various components of the flow cell. It is to be understood that these directional terms are not meant to imply a specific orientation, but are used to designate relative orientation between components. The use of directional terms should not be interpreted to limit the examples disclosed herein to any specific orientation(s).

The terms first, second, etc. also are not meant to imply a specific orientation or order, but rather are used to distinguish one component from another.

It is to be understood that the ranges provided herein include the stated range and any value or sub-range within the stated range, as if such values or sub-ranges were explicitly recited. For example, a range of about 400 nm to about 1 μm (1000 nm), should be interpreted to include not only the explicitly recited limits of about 400 nm to about 1 μm, but also to include individual values, such as about 708 nm, about 945.5 nm, etc., and sub-ranges, such as from about 425 nm to about 825 nm, from about 550 nm to about 940 nm, etc.

Furthermore, when “about” and/or “substantially” are/is utilized to describe a value, they are meant to encompass minor variations (up to +/−10%) from the stated value.

An “acrylamide monomer” is a monomer with the structure

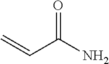

or a monomer including an acrylamide group. Examples of the monomer including an acrylamide group include azido acetamido pentyl acrylamide:

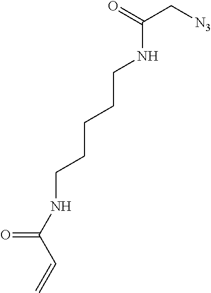 [SMILES: C=CC(=O)NCCCCCNC(=O)CN=[N+]=[N-]]

### and N-isopropylacrylamide:

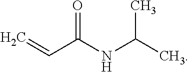

Other acrylamide monomers may be used.

An aldehyde, as used herein, is an organic compound containing a functional group with the structure-CHO, which includes a carbonyl center (i.e., a carbon double-bonded to oxygen) with the carbon atom also bonded to hydrogen and an R group, such as an alkyl or other side chain. The general structure of an aldehyde is:

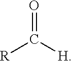

As used herein, “alkyl” refers to a straight or branched hydrocarbon chain that is fully saturated (i.e., contains no double or triple bonds). The alkyl group may have 1 to 20 carbon atoms. Example alkyl groups include methyl, ethyl, propyl, isopropyl, butyl, isobutyl, tertiary butyl, pentyl, hexyl, and the like. As an example, the designation “C1-4 alkyl” indicates that there are one to four carbon atoms in the alkyl chain, i.e., the alkyl chain is selected from the group consisting of methyl, ethyl, propyl, iso-propyl, n-butyl, isobutyl, sec-butyl, and t-butyl.

As used herein, “alkenyl” refers to a straight or branched hydrocarbon chain containing one or more double bonds. The alkenyl group may have 2 to 20 carbon atoms. Example alkenyl groups include ethenyl, propenyl, butenyl, pentenyl, hexenyl, and the like.

As used herein, “alkyne” or “alkynyl” refers to a straight or branched hydrocarbon chain containing one or more triple bonds. The alkynyl group may have 2 to 20 carbon atoms.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms. Examples of aryl groups include phenyl, naphthyl, azulenyl, and anthracenyl.

An “amine” or “amino” functional group refers to an —NRₐR_{b} group, where Rₐ and R_{b} are each independently selected from hydrogen (e.g.,

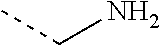

), C1-6 alkyl, C2-6 alkenyl, C2-6 alkynyl, C3-7 carbocycle, C6-10 aryl, 5-10 membered heteroaryl, and 5-10 membered heterocycle, as defined herein.

As used herein, the term “attached” refers to the state of two things being joined, fastened, adhered, connected or bound to each other, either directly or indirectly. For example, a nucleic acid can be attached to a functionalized polymer by a covalent or non-covalent bond. A covalent bond is characterized by the sharing of pairs of electrons between atoms. A non-covalent bond is a physical bond that does not involve the sharing of pairs of electrons and can include, for example, hydrogen bonds, ionic bonds, van der Waals forces, hydrophilic interactions and hydrophobic interactions.

An “azide” or “azido” functional group refers to —N₃.

As used herein, a “barcode” refers to a sequence of nucleotides attached to DNA molecules derived from the same DNA sample. The DNA sample is a large piece of DNA that is fragmented into smaller DNA molecules for analysis. The barcode length determines the plexity of the barcode pool and space.

As used herein, a “bonding region” refers to an area of a substrate that is to be bonded to another material, which may be, as examples, a spacer layer, a lid, another substrate, etc., or combinations thereof (e.g., a spacer layer and a lid, or a spacer layer and another substrate). The bond that is formed at the bonding region may be a chemical bond (as described above), or a mechanical bond (e.g., using a fastener, etc.).

As used herein, “carbocycle” means a non-aromatic cyclic ring or ring system containing only carbon atoms in the ring system backbone. When the carbocycle is a ring system, two or more rings may be joined together in a fused, bridged or spiro-connected fashion. Carbocycles may have any degree of saturation, provided that at least one ring in a ring system is not aromatic. Thus, carbocycles include cycloalkyls, cycloalkenyls, and cycloalkynyls. The carbocycle group may have 3 to 20 carbon atoms. Examples of carbocycle rings include cyclopropyl, cyclobutyl, cyclopentyl, cyclohexyl, cyclohexenyl, 2,3-dihydro-indene, bicyclo[2.2.2]octanyl, adamantyl, and spiro [4.4]nonanyl.

As used herein, the term “carboxylic acid” or “carboxyl” as used herein refers to —COOH.

As used herein, “cycloalkylene” means a fully saturated carbocycle ring or ring system that is attached to the rest of the molecule via two points of attachment.

As used herein, “cycloalkenyl” or “cycloalkene” means a carbocycle ring or ring system having at least one double bond, wherein no ring in the ring system is aromatic. Examples include cyclohexenyl or cyclohexene and norbornenyl or norbornene. Also as used herein, “heterocycloalkenyl” or “heterocycloalkene” means a carbocycle ring or ring system with at least one heteroatom in ring backbone, having at least one double bond, wherein no ring in the ring system is aromatic.

As used herein, “cycloalkynyl” or “cycloalkyne” means a carbocycle ring or ring system having at least one triple bond, wherein no ring in the ring system is aromatic. An example is cyclooctyne. Another example is bicyclononyne. Also as used herein, “heterocycloalkynyl” or “heterocycloalkyne” means a carbocycle ring or ring system with at least one heteroatom in ring backbone, having at least one triple bond, wherein no ring in the ring system is aromatic.

The term “depositing,” as used herein, refers to any suitable application technique, which may be manual or automated, and, in some instances, results in modification of the surface properties. Generally, depositing may be performed using vapor deposition techniques, coating techniques, grafting techniques, or the like. Some specific examples include chemical vapor deposition (CVD), spray coating (e.g., ultrasonic spray coating), spin coating, dunk or dip coating, doctor blade coating, puddle dispensing, flow through coating, aerosol printing, screen printing, microcontact printing, inkjet printing, or the like.

The term “each,” when used in reference to a collection of items, is intended to identify an individual item in the collection, but does not necessarily refer to every item in the collection. Exceptions can occur if explicit disclosure or context clearly dictates otherwise.

The term “epoxy” (also referred to as a glycidyl or oxirane group) as used herein refers to

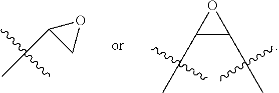

As used herein, the term “flow cell” is intended to mean a vessel having a flow channel where a reaction can be carried out, an inlet for delivering reagent(s) to the flow channel, and an outlet for removing reagent(s) from the flow channel. In some examples, the flow cell accommodates the detection of the reaction that occurs in the flow cell. For example, the flow cell can include one or more transparent surfaces allowing for the optical detection of arrays, optically labeled molecules, or the like.

As used herein, a “flow channel” or “channel” may be an area defined between two bonded components, which can selectively receive a liquid sample. In some examples, the flow channel may be defined between two substrates, and thus may be in fluid communication with surface chemistry of the substrates. In other examples, the flow channel may be defined between a substrate and a lid, and thus may be in fluid communication with surface chemistry of the substrates.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members.

As used herein, “heterocycle” means a non-aromatic cyclic ring or ring system containing at least one heteroatom in the ring backbone. Heterocycles may be joined together in a fused, bridged or spiro-connected fashion. Heterocycles may have any degree of saturation provided that at least one ring in the ring system is not aromatic. In the ring system, the heteroatom(s) may be present in either a non-aromatic or aromatic ring. The heterocycle group may have 3 to 20 ring members (i.e., the number of atoms making up the ring backbone, including carbon atoms and heteroatoms). In some examples, the heteroatom(s) are O, N, or S.

The term “hydrazine” or “hydrazinyl” as used herein refers to a —NHNH₂ group.

As used herein, the term “hydrazone” or “hydrazonyl” as used herein refers to a

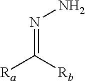

group in which Rₐ and R_{b} are each independently selected from hydrogen, C1-6 alkyl, C2-6 alkenyl, C2-6 alkynyl, C3-7 carbocycle, C6-10 aryl, 5-10 membered heteroaryl, and 5-10 membered heterocycle, as defined herein.

As used herein, “hydroxy” or “hydroxyl” refers to an —OH group.

“Nitrile oxide,” as used herein, means a “RₐC≡N⁺O⁻” group in which Rₐ is defined herein. Examples of preparing nitrile oxide include in situ generation from aldoximes by treatment with chloramide-T or through action of base on imidoyl chlorides [RC(Cl)═NOH] or from the reaction between hydroxylamine and an aldehyde.

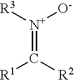

“Nitrone,” as used herein, means a group in which R¹, R², and R³ may be any of the Rₐ and R_{b} groups defined herein, except that R³ is not hydrogen (H).

As used herein, a “nucleotide” includes a nitrogen containing heterocyclic base, a sugar, and one or more phosphate groups. Nucleotides are monomeric units of a nucleic acid sequence. In RNA, the sugar is a ribose, and in DNA, the sugar is a deoxyribose, i.e. a sugar lacking a hydroxyl group that is present at the 2′ position in ribose. The nitrogen containing heterocyclic base (i.e., nucleobase) can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. A nucleic acid analog may have any of the phosphate backbone, the sugar, or the nucleobase altered. Examples of nucleic acid analogs include, for example, universal bases or phosphate-sugar backbone analogs, such as peptide nucleic acid (PNA).

In some examples, the term “over” may mean that one component or material is positioned directly on another component or material. When one is directly on another, the two are in contact with each other. In other examples, the term “over” may mean that one component or material is positioned indirectly on another component or material. By indirectly on, it is meant that a gap or an additional component or material may be positioned between the two components or materials.

As used herein, the “primer” is defined as a single stranded nucleic acid sequence (e.g., single strand DNA). Some primers, referred to herein as amplification primers, serve as a starting point for template amplification and cluster generation. Other primers, referred to herein as sequencing primers, serve as a starting point for DNA synthesis. The 5′ terminus of the primer may be modified to allow a coupling reaction with a functional group of a polymer. The primer length can be any number of bases long and can include a variety of non-natural nucleotides. In an example, the sequencing primer is a short strand, ranging from 10 to 60 bases, or from 20 to 40 bases.

A “spacer layer,” as used herein, refers to a material that bonds two components together. In some examples, the spacer layer can be a radiation absorbing material that aids in bonding, or can be put into contact with a radiation absorbing material that aids in bonding.

The term “substrate” refers to the single layer base support which surface chemistry is introduced.

A “thiol” functional group refers to —SH.

As used herein, the terms “tetrazine” and “tetrazinyl” refer to six-membered heteroaryl group comprising four nitrogen atoms. Tetrazine can be optionally substituted.

“Tetrazole,” as used herein, refers to a five-membered heterocyclic group including four nitrogen atoms. Tetrazole can be optionally substituted.

As used herein, “unique molecular indices” (UMIs) are sequences of nucleotides applied to DNA molecules that may be used to distinguish individual DNA molecules from one another. UMIs may be sequenced along with the DNA molecules with which they are associated to determine whether the read sequences are those of one source DNA molecule or another. UMIs allow for the grouping of DNA molecules in a cluster (i.e., amplicons of the DNA molecule) that are derived from the same source DNA molecule, but that are separately sequenced. The DNA molecule has adapters added thereto, and the complete molecule that is copied by amplification or otherwise to produce multiple instances is referred to herein as the “library template.”

UMIs are similar to barcodes (which are used to distinguish reads from one DNA sample from another DNA sample), except that UMIs instead are used to distinguish one source DNA molecule from another source DNA molecule when many DNA molecules are sequenced together. Because there may be many more DNA molecules in a sample than samples in a sequencing run, there are typically many more distinct UMIs than distinct barcodes in a sequencing run.

UMIs may be added to the DNA molecule. The UMIs are added to the DNA molecules by methods that physically link or bond the UMIs to the DNA molecules, e.g., by ligation or transposition through polymerase, endonuclease, transposases, etc.

“Unique dual indices” refer to two distinct, unrelated index adapters that are added to DNA molecules. In a DNA sample, a mixture of dual indices can be ligated to the DNA molecules. In the examples disclosed herein, these indices are used as a type of bar coding to help bioinformatically identify related reads (and thus narrowing the pool of reads for analysis), rather than sample differentiation.

Flow Cells with a Removable Blocking Mechanism

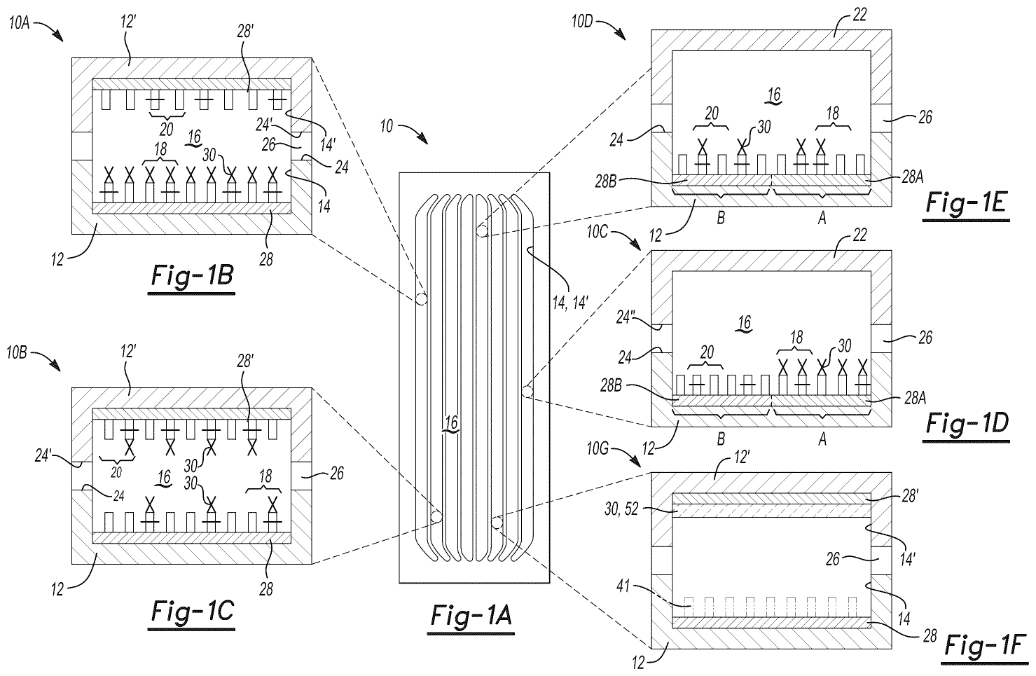

One example of a flow cell 10 is shown in FIG. 1A from a top view. Some of the different architectures within the flow cell 10 are shown in FIG. 1B through FIG. 1F.

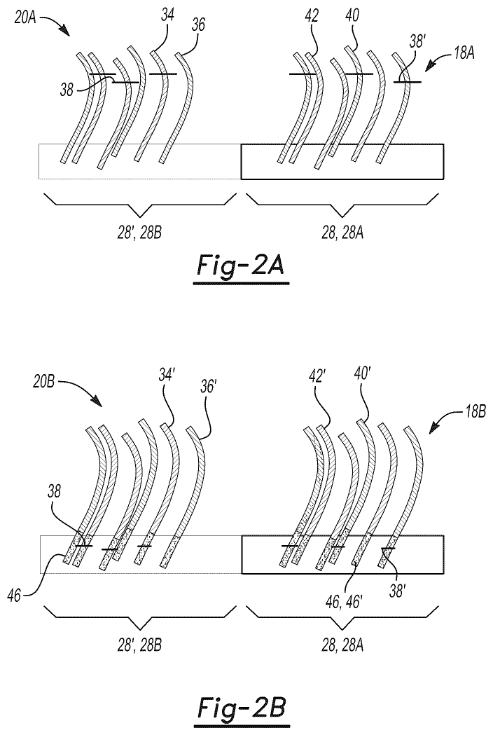

As shown in FIG. 1B and FIG. 1C, some examples of the flow cell 10A, 10B include a first substrate 12, a second substrate 12′ attached to the first substrate 12, a flow channel 16 defined between the first substrate 12 and the second substrate 12′, a first primer set 18 attached to the first substrate 12 (the first primer set 18, 18A, 18B including an un-cleavable first primer 42, 42′ and a cleavable second primer 40, 40′ (see FIG. 2A and FIG. 2B)), a second primer set 20 attached to the second substrate 12′ (the second primer set 20, 20A, 20B including a cleavable first primer 34, 34′ and an un-cleavable second primer 36, 36′ (see FIG. 2A and FIG. 2B)), and a removable blocking mechanism 30 passivating i) the first primer set 18 or the second primer set 20 (FIG. 1B) or ii) the cleavable first primer 34, 34′ and the cleavable second primer 40, 40′ (FIG. 1C).

As shown in FIG. 1D and FIG. 1E, other examples of the flow cell 10C, 10D include a substrate 12, lid 22 attached to the substrate 12, a flow channel 16 defined between the substrate 12 and the lid 22, a first primer set 18 attached to a first region A of the substrate 12 (the first primer set 18, 18A, 18B including an un-cleavable first primer 42, 42′ and a cleavable second primer 40, 40′ (see FIG. 2A and FIG. 2B)), a second primer set 20 attached to a second region B of the substrate 12 (the second primer set 20, 20A, 20B including a cleavable first primer 34, 34′ and an un-cleavable second primer 36, 36′ (see FIG. 2A and FIG. 2B)), and a removable blocking mechanism 30 passivating i) the first primer set 18 or the second primer set 20 (FIG. 1D) or ii) the cleavable first primer 34, 34′ and the cleavable second primer 40, 40′ (FIG. 1E).

Each substrate 12, 12′ is a base support. Examples of suitable base supports include epoxy siloxane, glass, modified or functionalized glass, polymers (including acrylics, polystyrene and copolymers of styrene and other materials, polypropylene, polyethylene, polybutylene, polyurethanes, polytetrafluoroethylene (such as TEFLON® from Chemours), cyclic olefins/cyclo-olefin polymers (COP) (such as ZEONOR® from Zeon), polyimides, etc.), and nylon (polyamides)), ceramics/ceramic oxides, silica, fused silica, or silica-based materials, aluminum silicate, silicon and modified silicon (e.g., boron doped p+ silicon), silicon nitride (Si₃N4), silicon oxide (SiO₂), tantalum pentoxide (Ta₂O₅) or other tantalum oxide(s) (TaOₓ), hafnium oxide (HfO₂), carbon, metals, inorganic glasses, resins, or the like.

The substrate 12, 12′ may be a circular or rectangular sheet, a panel, a wafer, a die, etc. having a diameter ranging from about 2 mm to about 300 mm, e.g., from about 200 mm to about 300 mm, or may be a rectangular sheet, panel, wafer, die etc. having its largest dimension up to about 10 feet (˜ 3 meters). For example, a die may have a width ranging from about 0.1 mm to about 10 mm. While example dimensions have been provided, it is to be understood that a substrate 12, 12′ with any suitable dimensions may be used.

When two opposed substrates 12, 12′ or 12, 12″ (see FIG. 7) are used, the substrate 12, 12′, or 12″ where a second round of seeding and amplification takes place may be larger than the substrate 12, 12′, 12″ where the first round of seeing and amplification takes place. The larger surface provides more space for amplicon complements to seed, thus increasing the likelihood of more completely representing the opposite stand (forward or reverse) of the amplicons generated during the first round of amplification.

The substrate 12, 12′ has a lane 14, 14′ defined therein. The lane 14, 14′ is a concave region in the substrate 12, 12′. The lane 14, 14′ may be defined via etching, imprinting, lithography, or another suitable technique.

The lane 14, 14′ may have any desirable shape. In an example, the lane 14, 14′ has a substantially rectangular configuration with curved ends (as shown in FIG. 1A). The length of the lane 14, 14′ depends, in part, upon the size of the substrate 12, 12′. The width of the lane 14, 14′ depends, in part, upon the size of the substrate 12, 12′, the desired number of lanes 14, 14′ formed in the substrate 12, 12′, the desired space between adjacent lanes 14, 14′, and the desired space at a perimeter of the substrate 12, 12′.

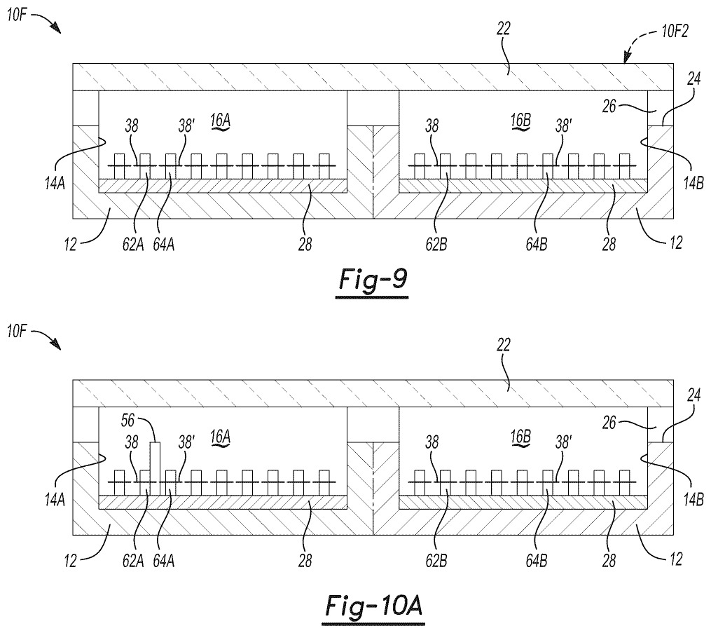

While not shown, any of the flow cells 10A, 10B, 10C, 10D (FIG. 1B through FIG. 1D), 10E (FIG. 7), 10F and 10F2 (FIG. 9), or 10G (FIG. 1F) disclosed herein may include depressions defined in the substrate(s) 12, 12′ instead of the lane 14, 14′. As opposed to a single lane 14, 14′ in fluid communication with the channel 16, multiple depressions are defined across the substrate 12, 12′ so that each individual depression is in fluid communication with the channel 16. When depressions are used, the substrate 12, 12′ may include multiple layers, such as the base support layer and a resin layer that overlies the base support layer and that has the depressions defined therein. The depressions may be defined via nanoimprint lithography, etching, or some other suitable patterning technique.

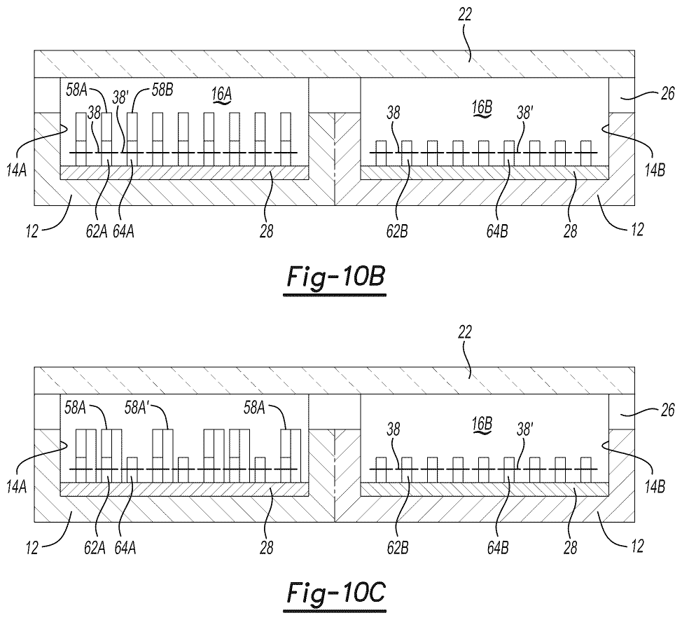

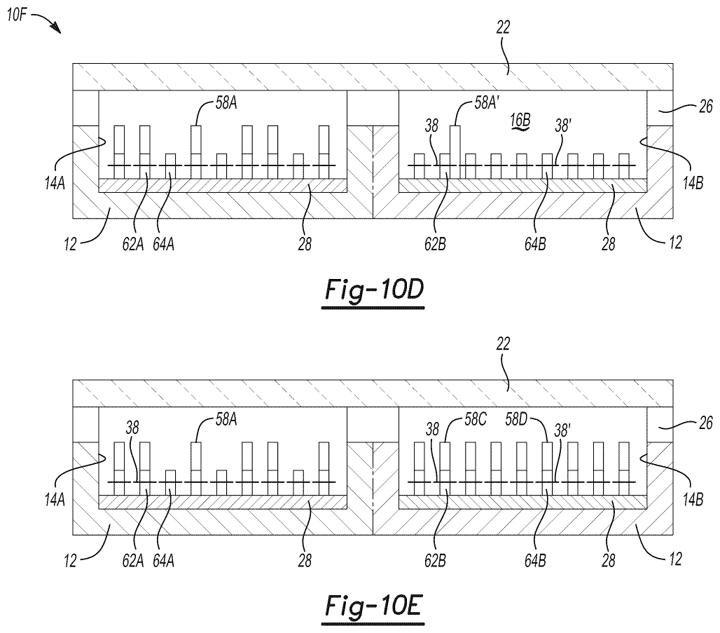

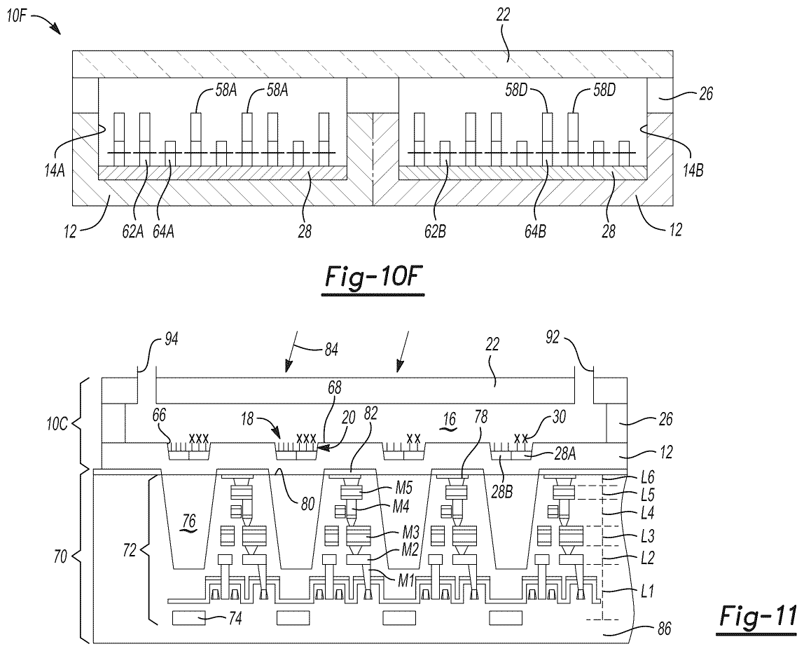

Depressions are individual wells that are capable of housing the polymeric hydrogel 28, 28′, 28A, 28B and the primer sets 18 or 20 or the primers 62, 64 (collectively referring to 62A, 62B, 64A, 64B, see FIG. 10). Each depression is separated by interstitial regions of the substrate (similar to the surfaces 24, 24′) that are free of the polymeric hydrogel 28, 28′, 28A, 28B and the primer sets 18 or 20 or the primers 62, 64.

Many different layouts of the depressions may be envisaged, including regular, repeating, and non-regular patterns. In an example, the depressions are disposed in a hexagonal grid for close packing and improved density. Other layouts may include, for example, rectilinear (rectangular) layouts, triangular layouts, and so forth. In some examples, the layout or pattern can be an x-y format in rows and columns. In some other examples, the layout or pattern can be a repeating arrangement of the depressions, each of which is separated by the interstitial regions.

The layout or pattern of depressions may be characterized with respect to the density (number) of the depressions in a defined area. For example, the depressions may be present at a density of approximately 2 million per mm². The density may be tuned to different densities including, for example, a density of about 100 per mm², about 1,000 per mm², about 0.1 million per mm², about 1 million per mm², about 2 million per mm², about 5 million per mm², about 10 million per mm², about 50 million per mm², or more, or less. It is to be further understood that the density can be between one of the lower values and one of the upper values selected from the ranges above, or that other densities (outside of the given ranges) may be used.

The layout or pattern of the depressions may also or alternatively be characterized in terms of the average pitch, or the spacing from the center of one depression to the center of an adjacent depression, or from the right edge of one depression to the left edge of an adjacent depression (edge-to-edge spacing). The pattern can be regular, such that the coefficient of variation around the average pitch is small, or the pattern can be non-regular in which case the coefficient of variation can be relatively large. In either case, the average pitch can be, for example, about 50 nm, about 0.1 μm, about 0.5 μm, about 1 μm, about 5 μm, about 10 μm, about 100 μm, or more or less.

The size of each depression may be characterized by at least one of its volume, opening area, depth, and/or diameter or length and width. For example, the volume can range from about 1×10−3 μm³ to about 100 μm³, e.g., about 1×10⁻² μm³, about 0.1 μm³, about 1 μm³, about 10 μm³, or more, or less. For another example, the opening area can range from about 1×10⁻³ μm² to about 100 μm², e.g., about 1×10⁻² μm², about 0.1 μm², about 1 μm², at least about 10 μm², or more, or less. For still another example, the depth can range from about 0.1 μm to about 100 μm, e.g., about 0.5 μm, about 1 μm, about 10 μm, or more, or less. For yet another example, the diameter or each of the length and the width of the depressions can range from about 0.1 μm to about 100 μm, e.g., about 0.5 μm, about 1 μm, about 10 μm, or more, or less.

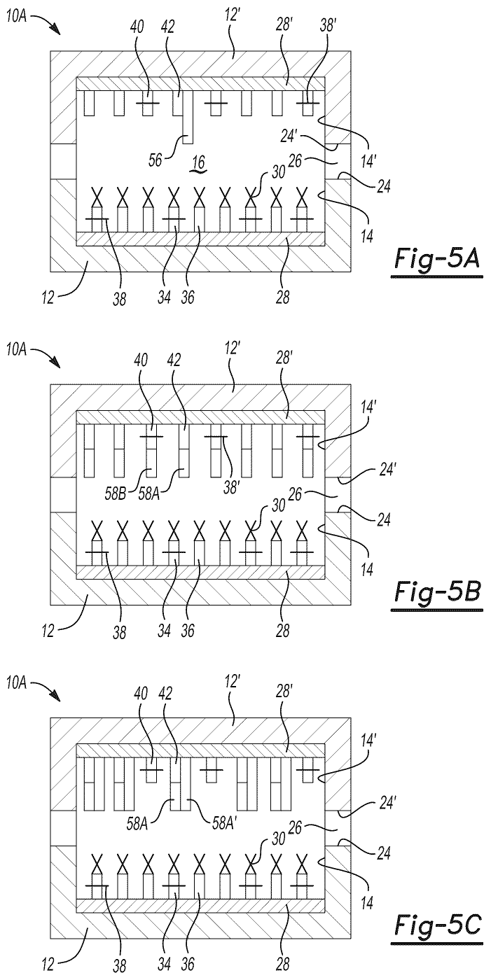

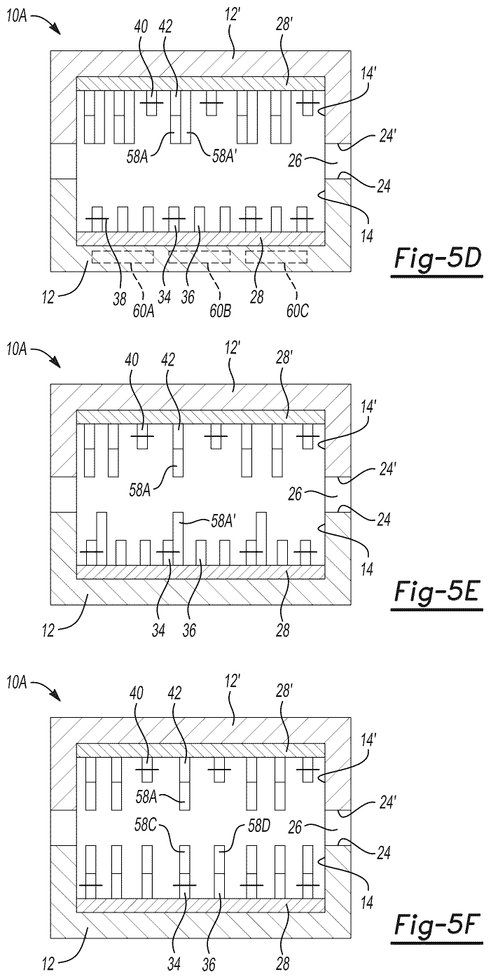

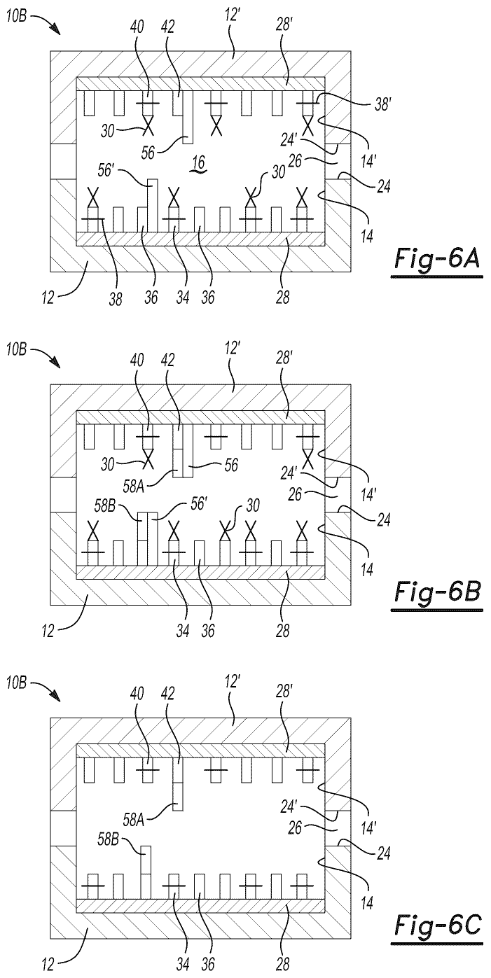

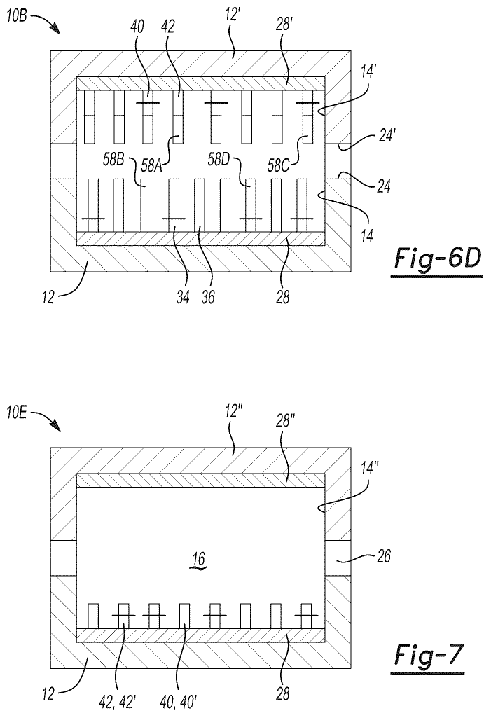

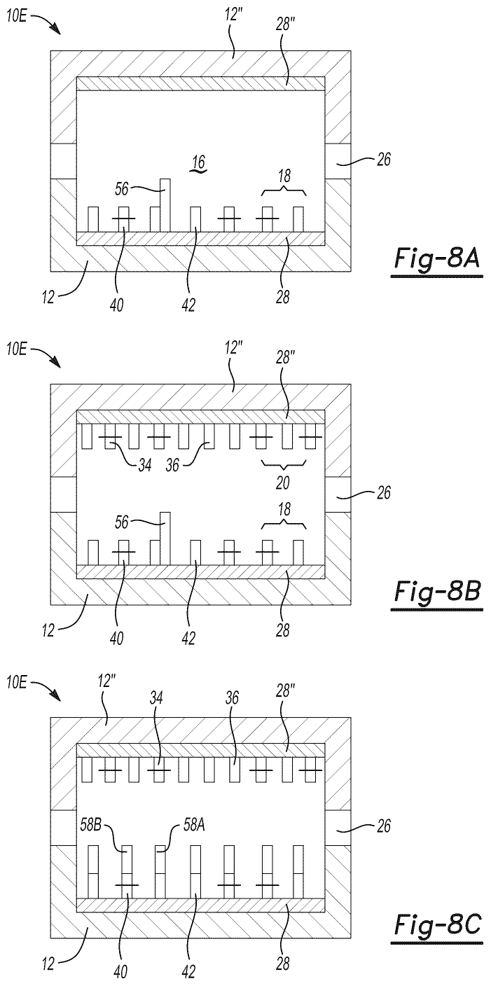

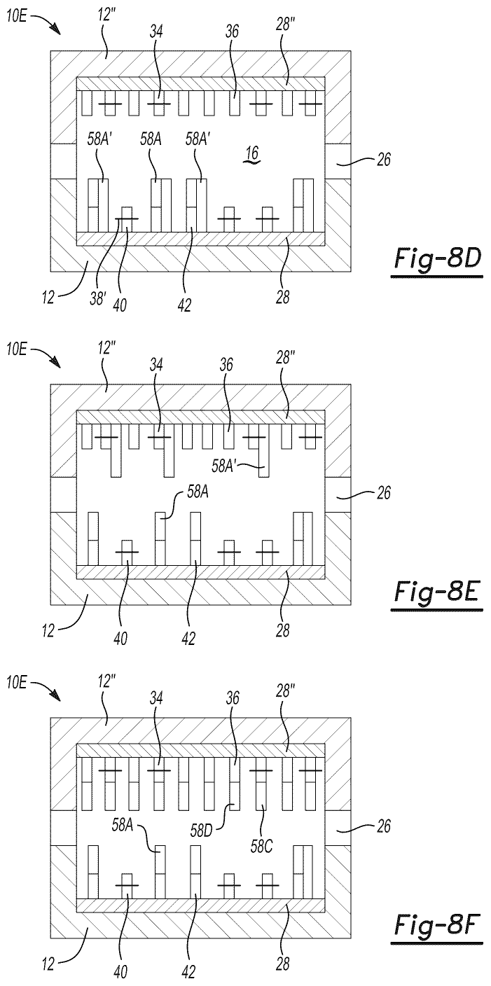

It is to be understood that when the depressions are used in the flow cells 10A, 10B, or 10E, the polymeric hydrogels 28, 28′ or 28, 28″ and primer sets 18, 20 will be respectively positioned within the depressions of the respective substrates 12, 12′ or 12, 12″. For visual reference, the lanes 14, 14′ shown in FIG. 1B, FIG. 1C, and FIG. 7 can represent one depression, and the substrates 12, 12′ would include several depressions separated by interstitial regions within a single channel 16. The methods described in reference to the FIG. 5 series, the FIG. 6 series, and the FIG. 8 series may be used with these patterned flow cells, where the reactions take place in the individual depressions.

Similarly, it is to be understood that when the depressions are used in the flow cell 10G, the polymeric hydrogel 28, 28′ and capture primers 41 will be respectively positioned within the depressions of the respective substrates 12, 12′. When the depressions are used in the flow cell 10F, the polymeric hydrogel 28 and primers 62A, 64A and 62B, 64B will be respectively positioned within the depressions formed where the lanes 14A, 14B are. For visual reference, the lanes 14, 14′ in FIG. 1F and the lanes 14A, 14B in FIG. 9 can represent one depression, and the substrates 12, 12′ would include several depressions separated by interstitial regions within a single channel 16, 16A, 16B. The rolling circle amplification methods or the method described in reference to the FIG. 6 series may be used with these patterned flow cells, where the reactions take place in the individual depressions.

It is to be further understood that when the depressions are used in the flow cells 10C and 10D, the polymeric hydrogels 28A, 28B and primer sets 18, 20 will be positioned within each of the depressions of the respective substrate (see FIG. 11, where the depressions are shown at reference numeral 66). For visual reference, the lanes 14, 14′ shown in FIG. 1D and FIG. 1E can represent one depression, and the substrates 12, 12′ would include several depressions separated by interstitial regions within a single channel 16. The methods described in reference to the FIG. 5 series and the FIG. 6 series, may be used with these patterned flow cells, where the reactions take place in the individual depressions.

The surface 24 of the substrate 12 surrounding each lane 14 or plurality of depressions may be sufficient for attachment to another substrate 12′ (FIG. 1B and FIG. 1C) or the lid 22 (FIG. 1D and FIG. 1E). The other substrate 12′ and the lid 22 also include surfaces 24′, 24″ that are sufficient for attachment to the substrate 12. Thus, these surfaces 24, 24′, 24″ are bonding regions.

To bond the other substrate 12′ or the lid 22 to the substrate 12, a spacer layer 26 may be used. The spacer layer 26 may be any material that will seal portions of the substrate 12 to the second substrate 12′ or to the lid 22. As examples, the spacer layer 26 may be an adhesive, a radiation-absorbing material that aids in bonding, or the like. In some examples, the spacer layer 26 is the radiation-absorbing material, e.g., KAPTON® black.

The substrate 12 and the second substrate 12′ or the lid 22 may be bonded using any suitable technique, such as laser bonding, diffusion bonding, anodic bonding, eutectic bonding, plasma activation bonding, glass frit bonding, or others methods known in the art.

When used, the lid 22 may be any material that is transparent to the excitation light that is directed toward the flow cell 10C, 10D. In optical detection systems, the lid 22 may also be transparent to the emissions generated from reaction(s) taking place in the flow cell 10C, 10D. As examples, the lid 22 may include glass (e.g., borosilicate, fused silica, etc.) or a transparent polymer. A commercially available example of a suitable borosilicate glass is D 2630, available from Schott North America, Inc.

Commercially available examples of suitable polymer materials, namely cyclo olefin polymers, are the ZEONOR® products available from Zeon Specialty Materials, Inc. In some instances, the lid 22 is shaped to form the top of the flow cell 10C, 10D, and in other instances, the lid 22 is shaped to form both the top of the flow cell 10C, 10D as well as sidewalls of the flow channel 16.

The spacer layer 26 is applied to or positioned on one or both of the surfaces 24 and/or 24′ or 24 and/or 24″ (i.e., the respective bonding regions of the substrate 12, 12′ and/or lid 22) where it seals the substrate 12 to the other substrate 12′ or to the lid 22. Within the flow cell 10, 10A, 10B, 10C, it is to be understood that bonding may take place at the perimeter of each lane 14, and at the perimeter of the flow cell 10, 10A, 10B, 10C.

In the examples shown in FIG. 1B and FIG. 1C, the primer sets 18, 20 are respectively attached to the different substrates 12, 12′. In the example shown in FIG. 1D and FIG. 1E, the primer sets 18, 20 are respectively attached to different regions A, B within the lane 14 of the substrate 12. In each of these examples, the primer sets 18, 20 are attached through a polymeric hydrogel 28, 28′ (FIG. 1B and FIG. 1C) or 28A, 28B (FIG. 1D and FIG. 1F).

The polymeric hydrogels 28, 28′ or 28A, 28B may be the same material or different materials, depending, in part, upon how the primer sets 18, 20 are to be grafted to the polymeric hydrogels 28, 28′ or 28A, 28B. In the examples of FIG. 1B and FIG. 1C, the polymeric hydrogel 28 is functionalized to attach one of the primer sets 18 or 20, and the other polymeric hydrogel 28′ is functionalized to attach the other of the primer sets 20 or 18. Similarly, in the example of FIG. 1D and FIG. 1E, the polymeric hydrogel 28A is functionalized to attach one of the primer sets 18 or 20, and the other polymeric hydrogel 28B is functionalized to attach the other of the primer sets 20 or 18. If the primer sets 18, 20 are pre-grafted to the respective polymeric hydrogels 28, 28′ or 28A, 28B, the polymeric hydrogels 28, 28′ or 28A, 28B may be the same or different because the formation of the two pre-grafted hydrogels takes place separately. Similarly, if the hydrogels 28, 28′ are applied to the substrates 12, 12′ and the primer sets 18, 20 are grafted to the hydrogels 28, 28′ before the substrates 12, 12′ are bonded, the polymeric hydrogels 28, 28′ may be the same or different because the addition of the surface chemistry to the respective substrates 12, 12′ takes place separately. In FIG. 1D and FIG. 1E, the polymer hydrogels 28A, 28B may be different materials, and the primer sets 18, 20 may include different 5′ end groups for respective attachment to the polymer hydrogels 28A, 28B. Alternatively, the polymeric hydrogels 28A, 28B may be the same materials, and masking techniques may be used during grafting to attach the desired primer set 18, 20 in the desired region A, B of the substrate 12. In another example, the polymeric hydrogels 28A, 28B can be the same material, which includes blocking groups that can be deblocked using light. The polymeric hydrogel 28A could be deblocked and grafted with one primer set 18 or 20, and then the other polymeric hydrogel 28B could be deblocked and grafted with the other primer set 20 or 18. In another example when the polymeric hydrogels 28A, 28B are the same material, temporary gasketing around the regions A or B could be used during the grafting reaction.

The polymeric hydrogels 28, 28′ or 28A, 28B may be any gel material that can swell when liquid is taken up and can contract when liquid is removed, e.g., by drying.

In an example, the polymeric hydrogel(s) 28, 28′ or 28A, 28B includes an acrylamide copolymer. Some examples of the acrylamide copolymer are represented by the following structure (I):

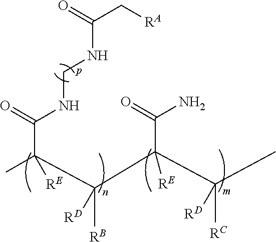

wherein:
R^{A} is selected from the group consisting of azido, optionally substituted amino, optionally substituted alkene, optionally substituted alkenyl, optionally substituted alkyne, halogen, optionally substituted hydrazone, optionally substituted hydrazine, carboxyl, hydroxy, optionally substituted tetrazole, optionally substituted tetrazine, nitrile oxide, nitrone, sulfate, and thiol;
R^{B} is H or optionally substituted alkyl;
R^{C}, R^{D}, and R^{E} are each independently selected from the group consisting of H and optionally substituted alkyl;
each of the —(CH₂)ₚ— can be optionally substituted;
p is an integer in the range of 1 to 50;
n is an integer in the range of 1 to 50,000; and
m is an integer in the range of 1 to 100,000. In structure (I), the R^{A} group may be selected to attach primers of the primer set(s) 18 and/or 20.

One specific example of the acrylamide copolymer represented by structure (I) is poly(N-(5-azidoacetamidylpentyl) acrylamide-co-acrylamide, PAZAM.

One of ordinary skill in the art will recognize that the arrangement of the recurring “n” and “m” features in structure (I) are representative, and the monomeric subunits may be present in any order in the polymer structure (e.g., random, block, patterned, or a combination thereof).

The molecular weight of the acrylamide copolymer may range from about 5 kDa to about 1500 kDa or from about 10 kDa to about 1000 kDa, or may be, in a specific example, about 312 kDa.

In some examples, the acrylamide copolymer is a linear polymer. In some other examples, the acrylamide copolymer is a lightly cross-linked polymer.

In other examples, the polymeric hydrogel(s) 28, 28′ or 28A, 28B may be a variation of structure (I). In one example, the acrylamide unit may be replaced with N,N-dimethylacrylamide

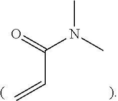

In this example, the acrylamide unit in structure (I) may be replaced with,

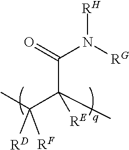

where R^{D}, R^{E}, and RF are each H or a C1-C6 alkyl, and RG and RH are each a C1-C6 alkyl(instead of H as is the case with the acrylamide). In this example, q may be an integer in the range of 1 to 100,000. In another example, the N,N-dimethylacrylamide may be used in addition to the acrylamide unit. In this example, structure (I) may include

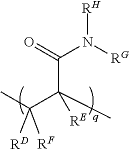

in addition to the recurring “n” and “m” features, where R^{D}, R^{E}, and R^{F} are each H or a C1-C6 alkyl, and RG and RH are each a C1-C6 alkyl. In this example, q may be an integer in the range of 1 to 100,000.

As another example of the polymeric hydrogel(s) 28, 28′ or 28A, 28B, the recurring “n” feature in structure (I) may be replaced with a monomer including a heterocyclic azido group having structure (II):

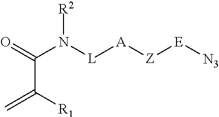

wherein R¹ is H or a C1-C6 alkyl; R² is H or a C1-C6 alkyl; L is a linker including a linear chain with 2 to 20 atoms selected from the group consisting of carbon, oxygen, and nitrogen and 10 optional substituents on the carbon and any nitrogen atoms in the chain; E is a linear chain including 1 to 4 atoms selected from the group consisting of carbon, oxygen and nitrogen, and optional substituents on the carbon and any nitrogen atoms in the chain; A is an N substituted amide with an H or a C1-C4 alkyl attached to the N; and Z is a nitrogen containing heterocycle. Examples of Z include 5 to 10 carbon-containing ring members present as a single cyclic structure or a fused structure. Some specific examples of Z include pyrrolidinyl, pyridinyl, or pyrimidinyl.

As still another example, the polymeric hydrogel(s) 28, 28′ or 28A, 28B may include a recurring unit of each of structure (III) and (IV):

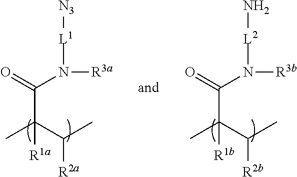

wherein each of R^{1a}, R^{2a}, R^{1b} and R^{2b} is independently selected from hydrogen, an optionally substituted alkyl or optionally substituted phenyl; each of R^{3a} and R^{3b} is independently selected from hydrogen, an optionally substituted alkyl, an optionally substituted phenyl, or an optionally substituted C7-C14 aralkyl; and each L¹ and L² is independently selected from an optionally substituted alkylene linker or an optionally substituted heteroalkylene linker.

In still another example, the acrylamide copolymer is formed using nitroxide mediated polymerization, and thus at least some of the copolymer chains have an alkoxyamine end group. In the copolymer chain, the term “alkoxyamine end group” refers to the dormant species —ONR₁R₂, where each of R₁ and R₂ may be the same or different, and may independently be a linear or branched alkyl, or a ring structure, and where the oxygen atom is attached to the rest of the copolymer chain. In some examples, the alkoxyamine may also be introduced into some of the recurring acrylamide monomers, e.g., at position R^{A} in structure (I). As such, in one example, structure (I) includes an alkoxyamine end group; and in another example, structure (I) includes an alkoxyamine end group and alkoxyamine groups in at least some of the side chains.

Another example of the polymeric hydrogel(s) 28, 28′ or 28A, 28B includes an ester copolymer having an NHS functional group for attachment to the primers of the primer set(s) 18 and/or 20. As another example, the polymeric hydrogel of structure (I) may include the NHS functional group as the R^{A} group.

While some example polymeric hydrogels 28, 28′ or 28A, 28B have been discussed, it is to be understood that the polymer structure may alternatively be a branched polymer, including dendrimers (e.g., multi-arm or star polymers, star-block polymers, and the like). For example, the monomers (e.g., acrylamide, etc.) may be incorporated, either randomly or in block, into the branches (arms) of a dendrimer.

In some examples, the surfaces (24, 24′ and those in the lanes 14, 14′) of the substrates 12, 12′ may activated, and then the polymeric hydrogel(s) 28, 28′ or 28A, 28B may be applied thereto. In one example, a silane or silane derivative (e.g., norbornene silane) may be deposited on the surfaces of the substrates 12, 12′ using vapor deposition, spin coating, or other deposition methods. In another example, the substrate surfaces may be exposed to plasma ashing to generate surface-activating agent(s) (e.g., —OH groups) that can adhere to the polymeric hydrogel 28, 28′ or 28A, 28B.

To introduce the polymeric hydrogels 28, 28′ or 28A, 28B into the lanes 14, 14′, mixtures of the respective polymeric hydrogels 28, 28′ may be generated and then applied to the respective substrates 12, 12′. In one example, the polymeric hydrogel 28, 28′ or 28A, 28B may be present in a mixture with water or with ethanol and water.

In the examples shown in FIG. 1B and FIG. 1C, the mixture may then be applied to the respective substrate surfaces (including in the lane 14, 14′) using spin coating, or dipping or dip coating, or flow of the material under positive or negative pressure, or another suitable technique. Other selective deposition techniques (e.g., involving a mask, controlled printing techniques, etc.) may be used to specifically deposit the polymeric hydrogel 28, 28′ in the lane 14, 14′ and not on the surfaces 24, 24′.

In the examples shown in FIG. 1D and FIG. 1E, the application of the polymeric hydrogels 28A, 28B will depend upon the chemistry of the hydrogels 28A, 28B. If the polymeric hydrogels 28A, 28B are the same material, any of the techniques described for the polymeric hydrogels 28, 28′ may be used, and then portions (e.g., at 28A or at 28B) of the hydrogel may be masked during primer grafting. If the polymeric hydrogels 28A, 28B are pre-grafted or are different materials, selective deposition techniques or masking techniques may be used to apply each of the polymeric hydrogels 28A, 28B in the desired regions of the lane 14. With one example masking technique, a developed photoresist may be to block a region A or B of the substrate 12 while one of the polymeric hydrogels 28A or 28B is applied to the other region B or A. Then, the photoresist may be removed (e.g., using a suitable developer) to expose the region A or B of the substrate 12. The other of the polymeric hydrogels 28B or 28A is then applied under high ionic strength conditions so that it attaches to the desired region A or B, but not to the already applied polymeric hydrogel 28A or 28B. With one example masking technique, one of the hydrogels 28A or 28B can be applied across the substrate 12, and then the photoresist can be developed over the hydrogel 28A or 28B in a region A or B so that a portion of the hydrogel 28A or 28B in another region B or A is exposed. While the developed photoresist is in place, the exposed portion of the hydrogel 28A or 28B is removed and the other of the polymeric hydrogels 28B or 28A is applied in the other region B or A. Then, the photoresist may be removed (e.g., using a suitable developer) to expose the originally applied polymeric hydrogel 28A or 28B. In still another example, the lane 14 may be patterned with two different silanes during activation, and the polymeric hydrogels 28A, 28B may respectively include functional groups that selective attach to the two different silanes. In this example, the polymeric hydrogels 28A, 28B can be simultaneously applied as they will respectively attach to the desired region A, B due to the different silanes.

Depending upon the chemistry of the polymeric hydrogel 28, 28′, 28A, 28B, the applied mixture may be exposed to a curing process. In an example, curing may take place at a temperature ranging from room temperature (e.g., about 25° C.) to about 95° C. for a time ranging from about 1 millisecond to about several days.

If the hydrogel(s) 28, 28′ or 28A, 28B is/are deposited on the surface 24, 24′, polishing may be performed in order to remove the polymeric hydrogel 28, 28′ or 28A, 28B from the surface 24, 24′ at the perimeter of the lane 14, 14′, while leaving the polymeric hydrogel 28, 28′ or 28A, 28B on the surface in the lane 14, 14′ at least substantially intact.

The flow cell architectures shown in FIG. 1B through FIG. 1E also include the primer sets 18, 20 respectively attached to the polymeric hydrogels 28, 28′ or 28A, 28B. Specific examples of the primers sets 18, 20 are shown in FIG. 2A as 18A, 20A and in FIG. 2B as 18B, 20B.

The primers sets 18A, 20A or 18B, 20B are related in that one set 20A, 20B includes a cleavable first primer 34, 34′ and an un-cleavable second primer 36, 36′ and the other set 18A, 18B includes an un-cleavable first primer 42, 42′ and a cleavable second primer 40, 40′. These primer sets 18A, 20A or 18B, 20B allow a single template strand to be amplified and clustered across both primer sets 18A, 20A or 18B, 20B, and also enable the respective generation of forward and reverse strands on the polymeric hydrogels 28, 28′ or 28A, 28B due to the cleavage sites 38, 38′ being present on the opposite primers of the sets 20A, 18A or 20B, 18B. It is to be understood that the prime (′) designations for the primers 34′, 36′, 40′, 42′ do not refer to complementary sequences to the primers 34, 36, 40, 42, but rather are additional examples of the type of primer.

Each of the first primer sets 18A, 18B includes an un-cleavable first primer 42 or 42′ and a cleavable second primer 40 or 40′; and each of the second primer sets 20A, 20B includes a cleavable first primer 34 or 34′ and an un-cleavable second primer 36 or 36′.

The un-cleavable second primer 36 or 36′ and the cleavable first primer 34 or 34′ are oligonucleotide pairs, e.g., where the un-cleavable second primer 36 or 36′ is a forward amplification primer and the cleavable first primer 34 or 34′ is a reverse amplification primer or where the un-cleavable second primer 36 or 36′ is the reverse amplification primer and the cleavable first primer 34 or 34′ is the forward amplification primer. In each example of the primer set 20A, 20B, the cleavable first primer 34 or 34′ includes a cleavage site 38, while the un-cleavable second primer 36 or 36′ does not include a cleavage site 38.

The cleavable second primer 40 or 40′ and the un-cleavable first primer 42 or 42′ are also oligonucleotide pairs, e.g., where the cleavable second primer 40 or 40′ is a forward amplification primer and the un-cleavable first primer 42 or 42′ is a reverse amplification primer or where the cleavable second primer 40 or 40′ is the reverse amplification primer and the un-cleavable first primer 42 or 42′ is the forward amplification primer. In each example of the primer set 18A, 18B, the cleavable second primer 40 or 40′ includes a cleavage site 38′, while the un-cleavable first primer 42 or 42′ does not include a cleavage site 38′.

It is to be understood that the un-cleavable second primer 36 or 36′ of the second primer set 20A, 20B and the cleavable second primer 40 or 40′ of the first primer set 18A, 18B, have the same nucleotide sequence (e.g., both are forward amplification primers), except that the cleavable second primer 40 or 40′ includes the cleavage site 38′ integrated into the nucleotide sequence or into a linker 46 or 46′ attached to the nucleotide sequence. Similarly, the cleavable first primer 34 or 34′ of the second primer set 20A, 20B and the un-cleavable first primer 42 or 42′ of the first primer set 18A, 18B have the same nucleotide sequence (e.g., both are reverse amplification primers), except that the cleavable first primer 34 or 34′ includes the cleavage site 38 integrated into the nucleotide sequence or into a linker 46 attached to the nucleotide sequence.

It is to be understood that when the first primers 34 and 42 or 34′ and 42′ are forward amplification primers, the second primers 36 and 40 or 36′ and 40′ are reverse primers, and vice versa.

The un-cleavable primers 36, 42 or 36′, 42′ may be any primers with a universal sequence for capture and/or amplification purposes, such as P5 and P7 primers, or any combination of PA, PB, PC, and PD primers (e.g., PA and PB or PA and PD, etc.).

Examples of the P5 and P7 primers are used on the surface of commercial flow cells sold by Illumina Inc. for sequencing, for example, on HISEQ™, HISEQX™, MISEQ™, MISEQDX™, MINISEQ™, NEXTSEQ™, NEXTSEQDX™, NOVASEQ™, NOVASEQX™, ISEQ™, GENOME ANALYZER™, and other instrument platforms. The un-cleavable P5 primer is:
un-cleavable P5: 5′→3′

(SEQ. ID. NO. 1)
AATGATACGGCGACCACCGAGACTACAC

The un-cleavable P7 primer may be any of the following:
un-cleavable P7 #1: 5′→3′

(SEQ. ID. NO. 2)
CAAGCAGAAGACGGCATACGAAT

un-cleavable P7 #2: 5′→3′

(SEQ. ID. NO. 3)
CAAGCAGAAGACGGCATACAGAT

The other un-cleavable primers (PA-PD) mentioned above include:
un-cleavable PA 5′→3′

(SEQ. ID. NO. 4)
GCTGGCACGTCCGAACGCTTCGTTAATCCGTTGAG

un-cleavable PB 5′→3′

(SEQ. ID. NO. 5)
CGTCGTCTGCCATGGCGCTTCGGTGGATATGAACT

un-cleavable PC 5′→3′

(SEQ. ID. NO. 6)
ACGGCCGCTAATATCAACGCGTCGAATCCGCAACT

un-cleavable PD 5′→3′

(SEQ. ID. NO. 7)
GCCGCGTTACGTTAGCCGGACTATTCGATGCAGC

These primers are un-cleavable primers 36, 42 or 36′, 42′ because they do not include a cleavage site 38, 38′. It is to be understood that any suitable universal sequence can be used as the un-cleavable primers 36, 42 or 36′, 42′.

Examples of cleavable primers 34, 40 or 34′, 40′ include the P5 and P7 primers or other universal sequence primers (e.g., the PA, PB, PC, PD primers) with the respective cleavage sites 38, 38′, incorporated into the respective nucleic acid sequences (FIG. 2A), or into the linker 46, 46′ (FIG. 2B) that attaches the cleavable primers 34, 40 or 34′, 40′ to the polymeric hydrogel 28′, 28 or 28B, 28A. Examples of suitable cleavage sites 38, 38′ include enzymatically cleavable nucleobases or chemically cleavable nucleobases, modified nucleobases, or other cleavable molecules (e.g., between nucleobases). Some specific examples of the cleavage sites 38, 38′ include uracil, 8-oxoguanine, alkene-thymidine, or allyl-T (a thymine nucleotide analog having an allyl functionality). The cleavage sites 38, 38′ may be incorporated at any point in the primer strand or in the linker 46, 46′. In the primer sets 18 (18A, 18B) and 20 (20A, 20B), the cleavage sites 38, 38′ are selected to be orthogonal. As such, the cleavage site 38 is not susceptible to the cleaving agent(s) used to cleave the cleavage site 38′, and vice versa.

Some specific examples of the cleavable primers 34, 40 or 34′, 40′ are shown below, where the cleavage site is uracil (U) or is shown at “n”:
cleavable P5 #1: 5′→3′

(SEQ. ID. NO. 8)
AATGATACGGCGACCACCGAGAUCTACAC

cleavable P5 #2: 5′→3′

(SEQ. ID. NO. 9)
AATGATACGGCGACCACCGAGAnCTACAC

wherein “n” is allyl T,

The cleavable P7 primer may be any of the following:
P7 #1: 5′→3′

(SEQ. ID. NO. 10)
CAAGCAGAAGACGGCATACGAnAT

P7 #2: 5′→3′

(SEQ. ID. NO. 11)
CAAGCAGAAGACGGCATACnAGAT

P7 #3: 5′→3′

(SEQ. ID. NO. 12)
CAAGCAGAAGACGGCATACnAnAT

where “n” is 8-oxoguanine in each of the sequences.

FIG. 2A and FIG. 2B depict different configurations of the primer sets 18A, 20A, 18B, 20B attached to the polymeric hydrogels 28, 28′ or 28A, 28B. More specifically, FIG. 2A and FIG. 2B depict different configurations of the primers 34, 36 or 34′, 36′ and 40, 42 or 40′, 42′ that may be used.

In the example shown in FIG. 2A, the primers 34, 36 and 40, 42 of the primer sets 20A, 18A are directly attached to the respective polymeric hydrogels 28′, 28 or 28B, 28A, for example, without a linker 46, 46′. The polymeric hydrogels 28′ and 28 or 28B and 28A have surface functional groups that can immobilize the terminal groups at the 5′ end of the respective primers 34, 36 and 40, 42.

Also, in the example shown in FIG. 2A, the cleavage site 38, 38′ of each of the cleavable primers 34, 40 is incorporated into the sequence of the primer 34, 40. It is to be understood that different types of cleavage sites 38, 38′ are used in the cleavable primers 34, 40 of the respective primer sets 20A, 18A. As an example, the cleavage site 38 is a uracil base (U) and the cleavage site 38′ is 8-oxoguanine (8OG), and the cleavable primers 34, 40 are P5-U and P7-8OG, respectively. It is to be understood that any other cleavable nucleotide that can be incorporated by a polymerase may be used as the cleavage sites 38, 38′. Any of the cleavage sites may also be incorporated into any of the PA, PB, PC, and PD primers to generate the cleavable primers 34, 40. In the example where the cleavable primers 34, 40 are P5-U and P7-8OG, the un-cleavable primer 36 of the oligonucleotide pair 34, 36 is P7, and the un-cleavable primer of the oligonucleotide pair 40, 42 is P5. Thus, in this example, the second primer set 20A includes P7, P5U and the first primer set 18A includes P5, P7-8OG.

In each of the examples, at least some of the cleavable primers 34, 40 in the primer sets 20A, 18A have opposite linearization chemistries, which contributes to the formation of forward template strands on one of the polymeric hydrogels 28′, 28 or 28B, 28A and reverse strands on the other the polymeric hydrogels 28, 28′ or 28A, 28B using the methods disclosed herein. There is one example, however, where some of the cleavable primers 34, 40 in the primer sets 20A, 18A have orthogonal linearization chemistries (i.e., cleavage sites 38 and 38′) and some other of the cleavable primers 34, 40 in the primer sets 20A, 18A have the same linearization chemistry (e.g., cleavage site 38 or 38′). The combination of the same and orthogonal linearization chemistries in the primer sets 20A, 18A may be used to adjust the concentration of amplicon complements that are dehybridized from one substrate 12, 12′ or region A, B and thus that are available for hybridization to the other substrate 12′, 12 or region B, A.

In the example shown in FIG. 2B, the primers 34′, 36′ and 40′, 42′ of the primer sets 20B and 18B are attached to the respective polymeric hydrogels 28′, 28 or 28B, 28A through linkers 46 or 46, 46′. The polymeric hydrogels 28′, 28B have surface functional groups that can immobilize the terminal groups of the linkers 46 at the 5′ end of the primers 34′, 36′. The polymeric hydrogels 28, 28A have surface functional groups that can immobilize the terminal groups of the linkers 46 or 46′ at the 5′ end of the primers 40′, 42′.

Examples of suitable linkers 46, 46′ may include nucleic acid linkers (e.g., 10 nucleotides or less) or non-nucleic acid linkers, such as a polyethylene glycol chain, an alkyl group or a carbon chain, an aliphatic linker with vicinal diols, a peptide linker, etc. An example of a nucleic acid linker is a polyT spacer, although other nucleotides can also be used. In one example, the spacer is a 6T to 10T spacer. The following are some examples of nucleotides including non-nucleic acid linkers with terminal alkyne groups (where B is the nucleobase and “oligo” is the primer sequence):

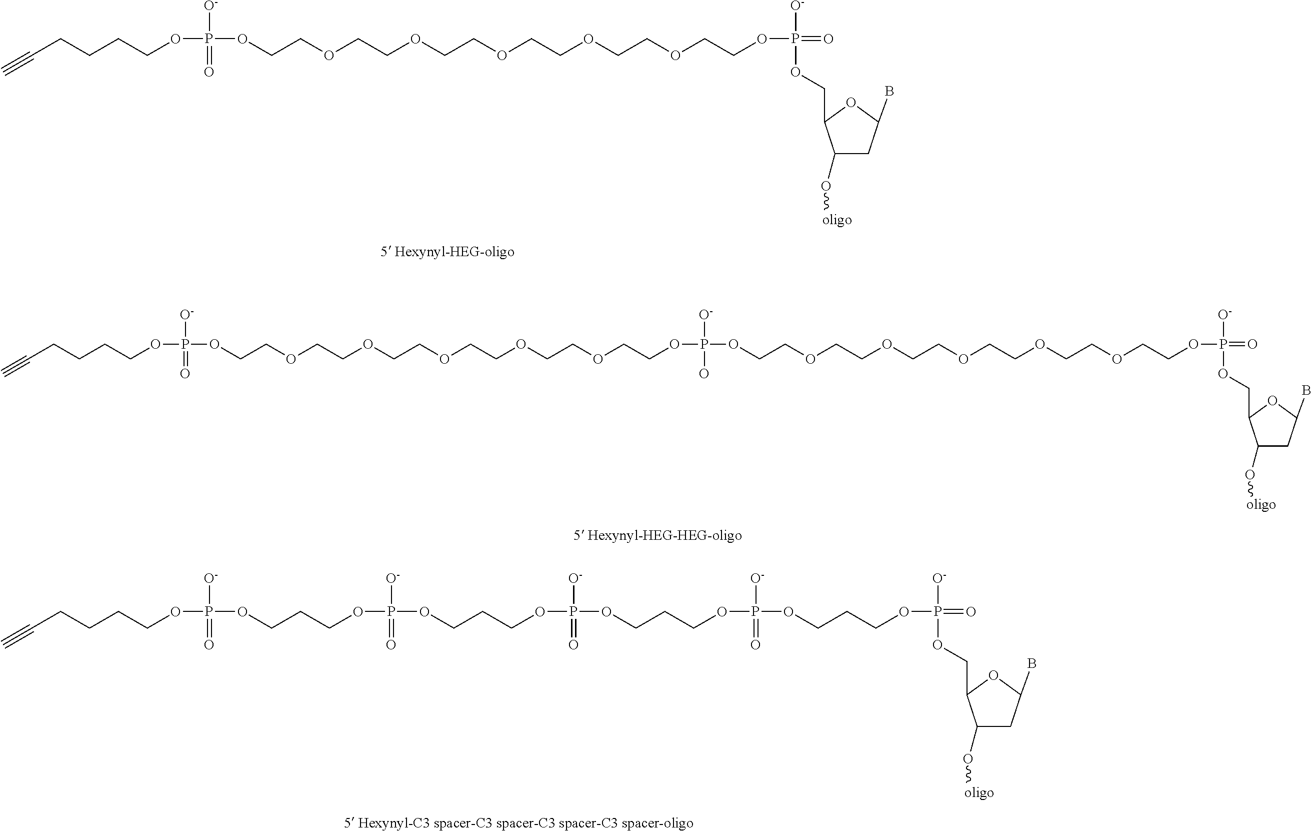

In the example shown in FIG. 2B, the primers 34′, 42′ have the same sequence (e.g., P5) and the same linkers 46 or different linkers 46, 46′. The primer 42′ is un-cleavable, whereas the primer 34′ includes the cleavage site 38 incorporated into the linker 46. Also in this example, the primers 36′, 40′ have the same sequence (e.g., P7) and the same linkers 46 or different linkers 46, 46′. The primer 36′ is un-cleavable, and the primer 40′ includes the cleavage site 38′ incorporated into the linker 46 or 46′. Different types of cleavage sites 38, 38′ are used in the linkers 46 or 46, 46′ of the cleavable primers 34′, 40′. In each of the examples, at least some of the cleavable primers 34, 40 in the primer sets 20B, 18B have opposite linearization chemistries, which, contributes to the formation of forward template strands on one of the polymeric hydrogels 28′, 28 or 28B, 28A and reverse strands on the other the polymeric hydrogels 28, 28′ or 28A, 28B using the methods disclosed herein. There is one example, however, where some of the cleavable primers 34′, 40′ in the primer sets 20B, 18B have orthogonal linearization chemistries (i.e., cleavage sites 38 and 38′) and some other of the cleavable primers 34′, 40′ in the primer sets 20B, 18B have the same linearization chemistry (e.g., cleavage site 38 or 38′). The combination of the same and orthogonal linearization chemistries in the primer sets 20B, 18B may be used to adjust the concentration of amplicon complements that are dehybridized from one substrate 12, 12′ or region A, B and thus that are available for hybridization to the other substrate 12′, 12 or region B, A.

While the cleavage sites 38, 38′ are shown as part of the linkers 46 or 46, 46′ in FIG. 2B, it is to be understood that the cleavage sites 38, 38′ of the primers 34′, 40′ may be incorporated into the primer sequence (at or near the 3′ end) rather than into the linkers 46 or 46, 46′.

As mentioned in the discussion of FIG. 2A and FIG. 2B, at least some of the cleavable primers 34, 40 or 34′, 40′ in the primer sets 20A, 18A or 20B, 18B have opposite/orthogonal linearization chemistries, and thus include different cleavage sites 38, 38′. In one example, the flow cell 10A or 10C includes a plurality of the first primer set 18A, 18B and a plurality of the second primer set 20A, 20B; where some of the cleavable first primers 34, 34′ of the plurality of the second primer set 20A, 20B include a first cleavage site 38 and some other of the cleavable first primers 34, 34′ of the plurality of the second primer set 20A, 20B include a second cleavage site 38′ that is orthogonal to the first cleavage site 38; and the cleavable second primers 40, 40′ of the plurality of the first primer set 18A, 18B include the second cleavage site 38′. In this particular example, the primer set 18 or 20 that is not initially blocked is the one whose cleavable primers 40, 40′ or 34, 34′ can include two orthogonal cleavage sites 38 and 38′. During examples of the method disclosed herein, the inclusion of primers 40, 40′ or 34, 34′ with two orthogonal cleavage sites 38 and 38′ will help to control the number of amplicon complements that are generated in the first round of amplification and thus that are available for hybridization prior to the second round of amplification. Within the single primer set 18 or 20, the number ratio of the cleavable primers 40, 40′ or 34, 34′ including the cleavage site 38 to those including the cleavage site 38′ may be altered to adjust the concentration of amplicon complements that can be formed during the first round of amplification.

In any of the primers 34, 34′, 36, 36′, 40, 40′, 42, 42′, the terminal groups at the 5′ end may be selected to attach to the desired polymeric hydrogel 28, 28′, 28A, 28B. Example 5′ end groups include an alkyne, an azide, an NHS-ester, a thiol, or any other functional group that is capable of single point attachment with a functional group of the polymeric hydrogel 28, 28′, 28A, 28B.

While the polymeric hydrogels 28 and 28′ or 28A and 28B are shown next to one another in FIG. 2A and FIG. 2B (similar to the architecture of FIG. 1D), it is to be understood that the polymeric hydrogels 28, 28′ will face one another as described in reference to FIG. 1B and FIG. 1C.

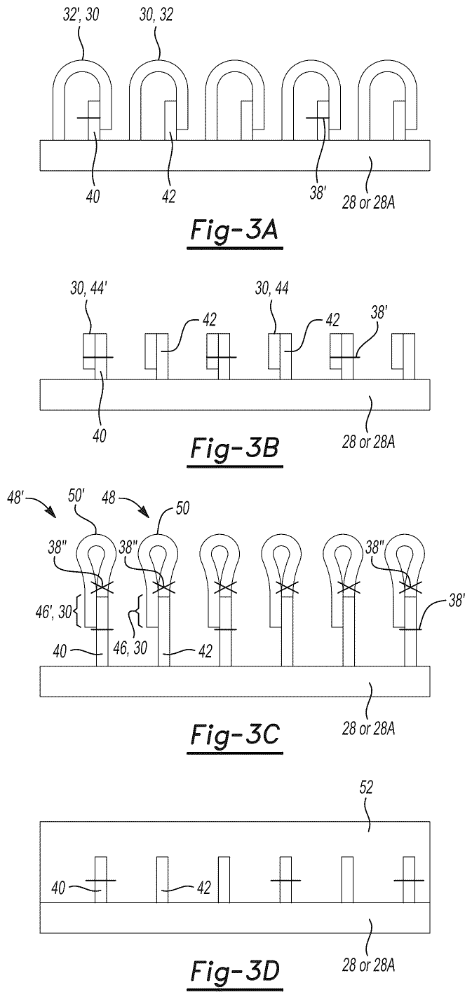

In some examples, one of the primer sets 18 or 20 that is initially incorporated into the flow cell 10A of FIG. 1B or the flow cell 10C of FIG. 1D is part of a linearizable hairpin primer set. The linearizable hairpin primers 48, 48′ in this primer set incorporate both the primers 34, 36 or 34′, 36′ or 40, 42, or 40′, 42′ and the removable blocking mechanism 30 into a single entity. An example of the linearizable hairpin primers 48, 48′ that are part of this set are shown and described in more detail in reference to FIG. 3C.

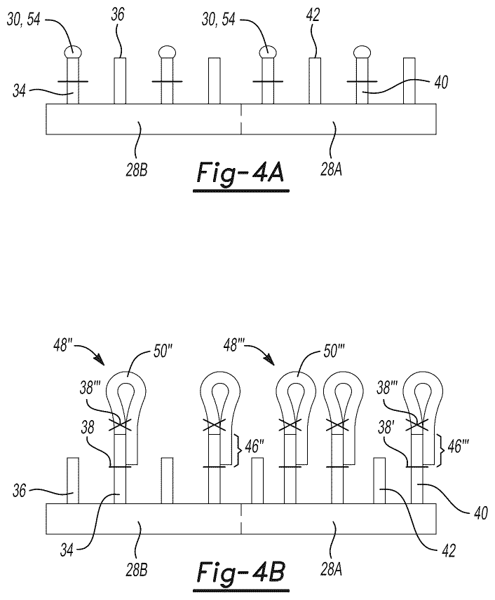

Each of the example flow cells 10A, 10B, 10C, 10D includes the removable blocking mechanism 30. Examples of the blocking mechanisms 30 for the flow cells 10A and 10C are shown in FIG. 3A through FIG. 3D, and examples of the blocking mechanisms 30 for the flow cells 10B and 10D are shown in FIG. 4A and FIG. 4B. For ease of illustration, it is to be understood that the substrate 12, 12′ or lid 22, and additional reference numbers, e.g., 34′, 36′, 40′, 42′, for the primer sets 20B, 18B are not shown in FIG. 3A through FIG. 4B. It is to be understood that any of the blocking mechanisms 30 described in these figures can be used with any of the primer sets 18A, 20A, 18B, 20B, and that the flow cells 10A, 10B, 10C, 10D incorporating the blocking mechanisms 30 include the components described herein in reference to FIG. 1A through FIG. 1E.

In FIG. 3A, the removable blocking mechanism 30 includes a plurality of linearizable blocking primers 32, 32′. The linearizable blocking primers 32, 32′ may be used in the flow cell 10A shown in FIG. 1B or the flow cell 10C shown in FIG. 1D.

When the linearizable blocking primers 32, 32′ of FIG. 3A are used in the flow cell 10A, the removable blocking mechanism 30 can passivate i) the first primer set 18 (e.g., 18A or 18B) or ii) the second primer set 20 (e.g., 20A or 20B); the removable blocking mechanism 30 includes a plurality of the linearizable blocking primers 32, 32′ grafted to the first substrate 12 or to the second substrate 12′ (e.g., through polymeric hydrogel 28 or 28′); and the plurality of linearizable blocking primers 32, 32′ is hybridized to the un-cleavable first primer 42, 42′ and the cleavable second primer 40, 40′ of the first primer set 18A, 18B or to the cleavable first primer 34, 34′ and the un-cleavable second primer 36, 36′ of the second primer set 20A, 20B. In the example shown in FIG. 3A, the plurality of linearizable blocking primers 32, 32′ is hybridized to the un-cleavable first primer 42 and the cleavable second primer 40 of the first primer set 18A, which are attached to the polymeric hydrogel 28. While not shown in FIG. 3A, it is to be understood that the other of the primer sets, e.g., 20, 20A, remains unblocked (as shown in FIG. 1B).

When the linearizable blocking primers 32, 32′ of FIG. 3A are used in the flow cell 10C, the removable blocking mechanism 30 can passivate i) the first primer set 18 (e.g., 18A or 18B) or ii) the second primer set 20 (e.g., 20A or 20B); the removable blocking mechanism 30 includes a plurality of the linearizable blocking primers 32, 32′ grafted to the first region A of the substrate 12 or to the second region B of the substrate 12 (e.g., through polymeric hydrogel 28A or 28B); and the plurality of linearizable blocking primers 32, 32′ is hybridized to the un-cleavable first primer 42, 42′ and the cleavable second primer 40, 40′ of the first primer set 18A, 18B or the cleavable first primer 34, 34′ and the un-cleavable second primer 36, 36′ of the second primer set 20A, 20B. In the example shown in FIG. 3A, the plurality of linearizable blocking primers 32, 32′ is hybridized to the un-cleavable first primer 42 and the cleavable second primer 40 of the first primer set 18A, which are attached to the polymeric hydrogel 28A. While not shown in FIG. 3A, it is to be understood that the other of the primer sets, e.g., 20A, remains unblocked (as shown in FIG. 1D).

The linearizable blocking primer 32 is capable of being grafted to the polymeric hydrogel 28, 28′, 28A, or 28B at its 5′ end. Any of the 5′ end functional groups described herein for the primers 40, 43, 34, 36 may be used. At its 3′ end, the linearizable blocking primer 32 has a single stranded sequence that is complementary to the first primers, i.e., the un-cleavable first primer 42, 42′ and the cleavable first primer 34, 34′. Between the first primer complementary sequence at the 3′ end and the 5′ end functional group, the linearizable blocking primer 32 includes a sequence that is non-complementary to the primers 40, 42, 34, 36 of the primer sets 18, 20. As such, the linearizable blocking primers 32 will not seed the subsequently introduced library template, which includes adapters at opposed ends that are respectively complementary to the primers 34 and 40 and to the primers 36 and 42. Any sequence that is non-complementary to the primers 40, 42, 34, 36 may be used for this portion of the linearizable blocking primer 32. Examples include poly A or poly T sequences.

The linearizable blocking primer 32′ is also capable of being grafted to the polymeric hydrogel 28, 28, 28A, or 28B at its 5′ end. Any of the 5′ end functional groups described herein for the primers 40, 43, 34, 36 may be used. At its 3′ end, however, the linearizable blocking primer 32′ has a single stranded sequence that is complementary to the second primers, i.e., the un-cleavable second primer 36, 36′ and the cleavable second primer 40, 40′. Between the second primer complementary sequence at the 3′ end and the 5′ end functional group, the linearizable blocking primer 32′ includes a sequence that is non-complementary to the primers 40, 42, 34, 36 of the primer sets 18, 20. As such, the linearizable blocking primers 32′ also will not seed the subsequently introduced library template, which includes adapters complementary to the primers 34 and 40 and to the primers 36 and 42.

Each of the linearizable blocking primers 32, 32′ also includes the same cleavage site 38 or 38′ as the cleavable primers 34 or 40 of the primer set 20 or 18 that is not passivated by the removable blocking mechanism 30. For example, when the linearizable blocking primers 32, 32′ are incorporated into the flow cell 10A as it is shown in FIG. 1B, the linearizable blocking primers 32, 32′ are hybridized to the primer set 18. In this example, the cleavage site of these linearizable blocking primers 32, 32′ is the same as the cleavage site 38 of the cleavable primer 34 of the unblocked primer set 20. In this particular example, the cleavage site 38′ of the cleavable primer 40 (which is blocked by the linearizable blocking primers 32′) is orthogonal to the cleavage site 38 of the linearizable blocking primers 32, 32′ and of the cleavable primer 34.

When used, the linearizable blocking primers 32, 32′ are grafted to the polymeric hydrogel 28 or 28′ or 28A or 28B in a similar manner as the primer set 18 or 20. To initiate hybridization of the linearizable blocking primers 32, 32′ to the respective primers 42, 40 or 34, 36, the grafted primers 32, 32′ and 42, 40 or 34, 36 are exposed to heat. During hybridization, the respective portion of the linearizable blocking primers 32, 32′ that is complementary to the primer 42 or 40 or 34 or 36 will hydrogen bond to the primer 42 or 40 or 34 or 36. As shown in FIG. 3A, the hybridized linearizable blocking primers 32, 32′ block the primer set 18, 18A.

Referring now to FIG. 3B, the removable blocking mechanism 30 includes a plurality of blocking primers 44, 44′. The blocking primers 44, 44′ may be used in the flow cell 10A shown in FIG. 1B or the flow cell 10C shown in FIG. 1D.

When the blocking primers 44, 44′ of FIG. 3B are used in the flow cell 10A or 10C, the removable blocking mechanism 30 can passivate i) the first primer set 18 (e.g., 18A or 18B) or ii) the second primer set 20 (e.g., 20A or 20B); the removable blocking mechanism 30 includes a plurality of the blocking primers 44, 44′; and the plurality of blocking primers 44, 44′ is hybridized to the un-cleavable first primer 42, 42′ and the cleavable second primer 40, 40′ of the first primer set 18A, 18B or to the cleavable first primer 34, 34′ and the un-cleavable second primer 36, 36′ of the second primer set 20A, 20B. In the example shown in FIG. 3B, the plurality of blocking primers 44, 44′ is passivating the first primer set 18, 18A and includes a first blocking primer 44 that is complementary to the un-cleavable first primer 42 and a second blocking primer 44′ that is complementary to the cleavable second primer 40. While not shown in FIG. 3B, it is to be understood that the other of the primer sets, e.g., 20, 20A, remains unblocked (as shown in FIG. 1B). It is to be understood that in another example, the plurality of blocking primers 44, 44′ is passivating the second primer set 20, 20A and includes a first blocking primer 44 that is complementary to the cleavable first primer 34 and a second blocking primer 44′ that is complementary to the un-cleavable second primer 36. In this example, the other of the primer sets, e.g., 18, 18A, remains unblocked.

As noted, the blocking primers 44, 44′ are capable of hybridizing, respectively, to the first primers 34, 42 and to the second primers 36, 40 (or vice versa). Thus, in one example, the blocking primer 44 has a single stranded sequence that is complementary to the first primers, i.e., the un-cleavable first primer 42, 42′ and the cleavable first primer 34, 34′, and the blocking primer 44′ has a single stranded sequence that is complementary to the second primers, i.e., the un-cleavable second primer 36, 36′ and the cleavable second primer 40, 40′. In another example, the blocking primer 44 has a single stranded sequence that is complementary to the first primers, i.e., the un-cleavable first primer 42, 42′ and the cleavable first primer 34, 34′, and the blocking primer 44′ has a single stranded sequence that is complementary to the second primers, i.e., the un-cleavable second primer 36, 36′ and the cleavable second primer 40, 40′.

When used, the blocking primers 44, 44′ are hybridized to the respective primers 42, 40 or 34, 36 after the primers 42, 40 or 34, 36 are grafted to the polymeric hydrogel 28 or 28′ or 28A or 28B (either before or after the polymeric hydrogel 28 or 28′ or 28A or 28B is applied to the substrate 12 or 12′ and before bonding to form the flow cell 10A or 10C). Hybridization can be initiated by heat exposure. During hybridization, the blocking primers 44, 44′ that are complementary to the primer 42 or 40 or 34 or 36 will hydrogen bond to the primer 42 or 40 or 34 or 36. As shown in FIG. 3B, the hybridized blocking primers 44, 44′ block the primer set 18, 18A.

Referring now to FIG. 3C, the removable blocking mechanism 30 includes hairpin formation sequences 46, 46′, which are a component of the linearizable hairpin primers 48, 48′. The linearizable hairpin primers 48, 48′, and thus hairpin formation sequences 46, 46′, may be used in the flow cell 10A shown in FIG. 1B or the flow cell 10C shown in FIG. 1D.

When hairpin formation sequences 46, 46′ of FIG. 3C are used in the flow cell 10A or 10C, the removable blocking mechanism 30 can passivate i) the first primer set 18 (e.g., 18A or 18B) or ii) the second primer set 20 (e.g., 20A or 20B). In the example specifically shown in FIG. 3C, the removable blocking mechanism 30 is passivating the first primer set 18 (specifically the primers 40, 42 or set 18A). In this example, the first primer set includes a first linearizable hairpin primer 48 and a second linearizable hairpin primer 48′; the first linearizable hairpin primer 48 includes the un-cleavable first primer 42 and a first hairpin portion 50 attached at a 3′ end of the un-cleavable first primer 42, the first hairpin portion 50 including a cleavage site 38″ that is orthogonal to a cleavage site 38′ of the cleavable second primer 40 and a first hairpin formation sequence 46 that is hybridized to at least a portion of the un-cleavable first primer 42; the second linearizable hairpin primer 48′ includes the cleavable second primer 40 and a second hairpin portion 50′ attached at a 3′ end of the cleavable second primer 40, the second hairpin portion 50′ including the cleavage site 38″ that is orthogonal to the cleavage site 38′ of the cleavable second primer 40 and a second hairpin formation sequence 46′ that is hybridized to at least a portion of the cleavable second primer 40. In this example, the removable blocking mechanism 30 includes both the first hairpin formation sequence 46 and the second hairpin formation sequence 46′.

While not shown, it is to be understood that when the removable blocking mechanism 30 includes both the first hairpin formation sequence 46 and the second hairpin formation sequence 46′, the blocking mechanism 30 can passivate the second primer set 20 (e.g., the primers 34, 36 or set 20A). In this example, the second primer set 20 includes a first linearizable hairpin primer 48 and a second linearizable hairpin primer 48′; the first linearizable hairpin primer 48 includes the cleavable first primer 34 and a first hairpin portion 50 attached at a 3′ end of the cleavable first primer 34, the first hairpin portion 50 including a cleavage site 38″ that is orthogonal to a cleavage site 38 of the cleavable first primer 34 and a first hairpin formation sequence 46 that is hybridized to at least a portion of the cleavable first primer 34; the second linearizable hairpin primer 48′ includes the un-cleavable second primer 36 and a second hairpin portion 50′ attached at a 3′ end of the un-cleavable second primer 36, the second hairpin portion 50′ including the cleavage site 38″ that is orthogonal to the cleavage site 38′ of the cleavable second primer 40 and a second hairpin formation sequence 46′ that is hybridized to at least a portion of the un-cleavable second primer 36. In this example, the removable blocking mechanism 30 includes both the first hairpin formation sequence 46 and the second hairpin formation sequence 46′.

In these examples, the hairpin portions 50, 50′ are attached at the 3′ ends of the respective primers 42, 40 of the primer set 18 or 20 that is passivated. The 5′ end of the hairpin portions 50, 50′ includes a single stranded sequence that is at least partially complementary to the respective primers 42, 40. This enables the hairpin portion 50, 50′ to loop over and form a hairpin configuration with the hybridized hairpin formation sequences 46, 46′ and primers 42, 40 forming the termini, as shown in FIG. 3C.

The portion of the linearizable hairpin primers 48, 48′ between the 3′ end of the primers 42, 40 and the hairpin formation sequences 46, 46′ may be any single stranded DNA sequence. This portion should not be complementary to sequencing adapters or other portions of the library template 56, such as read primer sites. In an example, the portion of the linearizable hairpin primers 48, 48′ between the 3′ end of the primers 42, 40 and the hairpin formation sequences 46, 46′ ranges in length from about 3 nucleotides to about 50 nucleotides.

The first and second hairpin portions 50, 50′ include the cleavage site 38″. This cleavage site 38″ may be any of the examples set forth herein, as long as it is orthogonal to the cleavage site 38′ or 38 of the cleavable primer 40 or 34 that is passivated. This cleavage site 38″ may be the same as or different than the cleavage site 38 or 38′ of the cleavable primer 34 or 40 that is not passivated by the blocking mechanism 30. When the cleavage site 38″ and the cleavage site 38 or 38′ of the unblocked cleavable primer 34 or 40 are the same, the linearization of the unblocked cleavable primer 34 or 40 (after seeding and amplification have taken place) and the removal of the linearizable hairpin primers 48, 48′ will take place simultaneously upon exposure to a single cleaving agent. When the cleavage site 38″ and the cleavage site 38 or 38′ of the unblocked cleavable primer 34 or 40 are different, the linearization of the unblocked cleavable primer 34 or 40 (after seeding and amplification have taken place) and the removal of the linearizable hairpin primers 48, 48′ will take place sequentially upon exposure to two different cleaving agents.

When used, the linearizable hairpin primers 48, 48′ are grafted to the polymeric hydrogel 28 or 28′ or 28A or 28B in a similar manner as the primer set 18 or 20. To initiate hybridization of the hairpin formation sequences 46, 46′ to the respective primers 42, 40 or 34, 36, the grafted linearizable hairpin primers 48, 48′ are exposed to heat. During hybridization, the hairpin formation sequences 46, 46′ that are respectively complementary to the primers 42, 40 or 34, 36 will hydrogen bond to the primers 42, 40 or 34, 36. As shown in FIG. 3C, the hairpin formation block primers the primer set 18, 18A.

In still another example, the removable blocking mechanism 30 is passivating i) the first primer set 18 or the second primer set 20; and, as shown in FIG. 3D, the removable blocking mechanism 30 is a removable hydrophobic barrier layer 52 overlying the first primer set 18 or the second primer set 20.

Examples of the removable hydrophobic barrier layer 52 are selected from the group consisting of a fluorinated polymer, a perfluorinated polymer, a silicon polymer, and a mixture thereof. As examples, the removable hydrophobic barrier layer 52 may include an amorphous fluoropolymer (commercially available examples of which include those in the CYTOP® series from AGC Chemicals, which have one of the following terminal functional groups: A type:—COOH or S type: —CF₃, some of which can be dissolved in fluorinated solvents), parylene C (which can be dissolved in chloro-naphthalene at 175° C.), a fluoroacrylic copolymer (a commercially available example of which includes as FLUOROPEL® from Cytonix, which can be dissolved in several fluorosolvents).

The hydrophobic barrier layer 52 may be formed over the primer set 18 or 20 by depositing the hydrophobic material over the grafted primer set 18 or 20 and then drying or curing the applied hydrophobic material. This example of the removable blocking mechanism 30 may be applied before the substrate 12 is bonded to the second substrate 12′ or before or after the substrate 12 is bonded to the lid 22.

The flow cells 10B and 10D include the blocking mechanism 30 on the cleavable primers 34, 40 or 34′, 40′. As mentioned, examples of the blocking mechanisms 30 for these flow cells 10B and 10D are shown in FIG. 4A and FIG. 4B.

In FIG. 4A, the removable blocking mechanism 30 is passivating the cleavable first primer 34, 34′ and the cleavable second primer 40, 40′; and the removable blocking mechanism 30 is a terminal phosphate group 54 at a 3′ end of each of the cleavable first primer 34, 34′ and the cleavable second primer 40, 40′. These terminal phosphate groups 54 can prevent extension of the cleavable primers 34, 34′ and 40, 40′ when library templates that are hybridized to the un-cleavable primers 36, 36′ and 42, 42′ are extended. The terminal phosphate groups 54 may then be dephosphorylated as described in more detail in reference to the methods.

Another example of the blocking mechanism 30 for the flow cells 10B and 10D is shown in FIG. 4B. In this example, the removable blocking mechanism 30 is passivating the cleavable first primer 34, 34′ and the cleavable second primer 40, 40′; the cleavable first primer 34, 34′ is a part of a first linearizable hairpin primer 48″ including a first hairpin portion 50″ attached at a 3′ end of the cleavable first primer 34, 34′, where the first hairpin portion 50″ includes a cleavage site 38′″ that is orthogonal to a cleavage site 38, 38′ of each of the cleavable first primer 34, 34′ and the cleavable second primer 40, 40′ and a first hairpin formation sequence 46″ that is hybridized to at least a portion of the cleavable first primer 34, 34′; and the cleavable second primer 40, 40′ is a part of a second linearizable hairpin primer 48″ including a second hairpin portion 50′″ attached at a 3′ end of the cleavable second primer 40, 40′, the second hairpin portion 50″ including the cleavage site 38″ that is orthogonal to a cleavage site 38, 38′ of each of the cleavable first primer 34, 34′ and the cleavable second primer 40, 40′, and a second hairpin formation sequence 46′″ that is hybridized to at least a portion of the cleavable second primer 40, 40′.

In this example, the removable blocking mechanism 30 includes both the first hairpin formation sequence 50″ and the second hairpin formation sequence 50″. The hairpin formation sequences 50″, 50″ are similar to the hairpin formation sequences 50, 50′ except that they are attached to the cleavable primers 34, 34′ and 40, 40′ of the primers sets 20, 18 as opposed to being attached to the cleavable and un-cleavable primers 34, 36 or 40, 42 of a single primer set 20 or 18.

Still another example of a flow cell 10G is depicted in FIG. 1F. This figure depicts two examples of the flow cell 10G, one of which is suitable for use with rolling circle amplification (RCA) products generated off-board the flow cell 10G (i.e., no capture primers 41 are present), and the other of which is suitable for generating RCA products on-board the flow cell 10G (i.e., capture primers 41 are present).

The flow cell 10G includes two opposed patterned substrates 12, 12′ that include the polymeric hydrogel 28, 28′ in the lane 14, 14′ or depressions. In these examples, the polymeric hydrogel 28, 28′ is cationic in order to electrostatically seed RCA products and/or complements thereof.

The flow cell 10G includes the hydrophobic barrier layer 52 as the removable blocking mechanism 30. This blocking mechanism 30 covers the polymeric hydrogel 28′ that is to receive the RCA product complements (as described in the methods involving RCA below).

When the flow cell 10G is to receive RCA products generated off-board the flow cell 10G, no capture primers 41 are present. Thus, in this example, the polymeric hydrogel 28 would be exposed to receive and electrostatically seed the RCA products.

Alternatively, when the flow cell 10G is used to generate the RCA products, capture primers 41 are grafted to the polymeric hydrogel 28. The capture primers 41 have any suitable universal sequence for DNA capture, and are able to seed cyclic DNA templates. Examples of suitable capture primers 41 include:
PX 5′→3′

(SEQ. ID. NO. 13)
AGGAGGAGGAGGAGGAGGAGGAGG.

Different methods for using the flow cells 10A, 10B, 10C, 10D, 10G will now be described.

Methods for Using Flow Cells with the Removable Blocking Mechanism

The flow cells 10A, 10B, 10C, 10D can be used for simultaneous paired-end sequencing. With the flow cells 10A and 10C, one of the primer sets 18 or 20 is blocked while library templates are seeded and amplified using the other of the primer sets 20 or 18. With the flow cells 10B and 10D, the cleavable primers 34, 34′, 40, 40′ are blocked while library templates are seeded using the un-cleavable primers 36, 36′, 42, 42′.

Preparation of the library templates may take place off board the flow cells 10A, 10B, 10C, 10D. The library templates may be prepared from any nucleic acid sample (e.g., a DNA sample or an RNA sample).

In one example, the DNA nucleic acid sample may be fragmented into single-stranded, similarly sized (e.g., <1000 bp) DNA fragments. In another example, the RNA nucleic acid sample may be used to synthesize complementary DNA (cDNA), and the cDNA may be fragmented into single-stranded, similarly sized (e.g., <1000 bp) cDNA fragments. The DNA fragments and cDNA fragments are examples of the DNA molecules described herein. During preparation, adapters may be added to the ends of any of the fragments. Through reduced cycle amplification or another suitable technique, different motifs may be introduced in the adapters, such as sequencing primer binding sites, a unique molecular index (UMI) or unique dual indices (UDI), and regions that are complementary to the primers 34, 42 or 36, 40. In some examples, the fragments from a single nucleic acid sample have the same adapters added thereto but have different UMIs or UDIs added thereto. The final library templates include the DNA or cDNA fragment (i.e., DNA molecule) and adapters at both ends. The DNA or cDNA fragment represents the portion of the final library template that is to be sequenced.

As mentioned, the library templates may each include sequencing primer binding site(s). In some instances, is may be desirable to include several sequencing primer binding sites, such as one to initiate a read of the DNA molecule and one to initiate a read of the UMI or respective ones to initiate reads of the respective indices of a UDI. The sequencing primer binding sites can bind sequencing primers for initiating a first read of the DNA molecule (i.e., a Read 1 primer), for initiating a second read of the DNA molecule (i.e., a Read 2 primer), and for initiating reads of the UMI or UDIs (i.e., index primers).

Also mentioned, the library templates may each include a UMI or unique dual indices (UDI). The UMI is uniquely associated with a single DNA molecule (which is a portion of the library template) and its amplicons. Thus, the UMI is used to link clusters of amplicons that are generated from the same library template and thus the same source DNA molecule, but that are respectively generated on the substrates 12, 12′ or the regions A, B using the method disclosed herein. The UDI is uniquely associated with several DNA molecules of a sample. Thus, the UDI is used to link clusters of amplicons that are generated from the same DNA sample, but that are respectively generated on the substrates 12, 12′ or the regions A, B using the method disclosed herein.

Each UMI is an oligonucleotide sequence having a sufficient length to ensure uniqueness for each and every source DNA molecule in a sample. In one example, the UMI has from 6 nucleotides to 40 nucleotides. Alternate example ranges include from 10 nucleotides to 35 nucleotides and from 15 nucleotides to 30 nucleotides. Each index adapter of a UDI is an oligonucleotide sequence having a length fewer than 12 nucleotides. The index adapters of a UDI together provide a type of bar coding for the DNA molecules in a sample. In some examples, each UMI or each index adapter of a UDI includes from 2 to no more than 6 nucleotides, or from 2 to no more than 4 nucleotides.

UMIs may be random, pseudo-random or partially random, or non-random nucleotide sequences that are inserted in adapters or otherwise incorporated in library templates to be sequenced. In some examples, the UMIs may be so unique that each of them is expected to uniquely identify any given library template present in a sample. The collection of adapters is generated, each having a UMI, and those adapters are attached to library templates to be sequenced. As such, each individually sequenced template has a UMI that helps distinguish it from all other templates. In such implementations, a very large number of different UMIs (e.g., many thousands to millions) may be used to uniquely identify DNA fragments in a sample.

A “random UMI” may be considered a UMI selected as a random sample, with or without replacement, from a set of UMIs consisting of all possible different oligonucleotide sequences given one or more sequence lengths. For instance, if each UMI in the set of UMIs has n nucleotides, then the set includes 4 n UMIs having sequences that are different from each other. A random sample selected from the 4 n UMIs constitutes a random UMI.

Conversely, a “nonrandom UMI” is UMI that is not a random UMI. Nonrandom UMIs may be predefined for a particular experiment or application, e.g., using a set of rules. As one example, every nonrandom UMI differs from every other nonrandom UMI by at least two nucleotides at corresponding sequence positions of the nonrandom UMIs.

In addition to or in place of a UMI, a randomer could be introduced to a library template via CRISPR or mutagenesis. Rather than a synthesized UMI, this randomer is a mutated form of the existing DNA.

The library templates may be used in any of the flow cells 10A, 10B, 10C, or 10D. The method shown in FIG. 5A through FIG. 5F utilizes the flow cells 10A or 10C. Flow cell 10A is shown in the FIG. 5 series. In the example shown in FIG. 5A through FIG. 5F, the first primer set 18 includes the un-cleavable first primer 42, 42′ and the cleavable second primer 40, 40′; the second primer set 20 includes the cleavable first primer 34, 34′ and the un-cleavable second primer 36, 36′; and the cleavable first primer 34, 34′ and the un-cleavable second primer 36, 36′ of the second primer set 20 are passivated by the removable blocking mechanism 30. The method may alternatively be performed where the primers 40, 40′, 42, 42′ of the first primer set 18 are passivated at the outset. For ease of illustration, it is to be understood that the additional reference numbers, e.g., 34′, 36′, 40′, 42′, for the primer sets 20B, 18B are not shown in FIG. 5A through FIG. 5F, but these primers 34′, 36′, 40′, 42′ could be used.

This method involves introducing a library template 56 into the flow cell 10A or 10C (which includes spatially separated first and second primer sets 18, 20 as described herein), whereby the library template 56 seeds to one of the primers 40, 40′ or 42, 42′ of the first primer set 18 (FIG. 5A); initiating amplification of the library template 56 using the first primer set 18 while the cleavable first primer 34, 34′ and the un-cleavable second primer 36, 36′ of the second primer set 20 are passivated, thereby generating a first amplicon 58A attached to the un-cleavable first primer 42, 42′ and a second amplicon 58B attached to the cleavable second primer 40, 40′ of the first primer set 18 (FIG. 5B); cleaving the second amplicon 58B from the cleavable second primer 40, 40′ (FIG. 5C); performing an extension reaction along the first amplicon 58A to generate a first amplicon complement 58A′ (FIG. 5C); removing the removable blocking mechanism 30 from the cleavable first primer 34, 34′ and the un-cleavable second primer 36, 36′ of the second primer set 20 (FIG. 5D); dehybridizing the first amplicon complement 58A′ from the first amplicon 58A (FIG. 5E); and initiating seeding of the first amplicon complement 58A′ to one of the primers, e.g., cleavable first primer 34, 34′, of the second primer set 20 (FIG. 5E). FIG. 5F depicts the amplicons 58C, 58D generated from the seeded and amplified first amplicon complements 58A′.

In FIG. 5A, the library template 56 is introduced into the flow cell 10A or 10C. While a single library template 56 is shown, it is to be understood that a plurality of different library templates (e.g., generated from a single DNA sample) may be introduced into the flow cell 10A or 10C together. In this example, the different library templates 56 will seed at random unblocked primers, e.g., un-cleavable first primer 42, 42′ across the substrate 12 or 12′. While not shown, it is to be understood that library template 56 seeding may also occur at random blocked primers depending on the blocking mechanism 30 that is used, but extension will not occur due to the removable blocking mechanism 30.

Prior to introduction into the flow cell 10A or 10C, the plurality of library templates 56 may be introduced to a suspension, which includes a liquid carrier (e.g., water and/or an ionic salt buffer). The library template suspension is then introduced into the flow cell 10A or 10C, where the library templates 56 are respectively hybridized, for example, to one of unblocked primers, e.g., 40, 40′ or 42, 42′, immobilized within the flow channel 16. The flow cell 10A or 10C, or at least the contents within the flow cell 10A or 10C, is/are at or is/are increased to a suitable hybridization temperature. As depicted in FIG. 5A, in this example, the library templates 56 do not hybridize to the blocked primers 34, 34′ and 36, 36′.

Amplification of the seeded library templates 56 is initiated to form clusters of the library templates 56. Each cluster includes amplicons 58A, 58B that are respectively attached to the primers 42, 42′ and 40, 40′. When multiple library templates 56 are seeded across the lane 14 or 14′ of the substrate 12 or 12′, the size of each cluster that is generated and the number of amplicons 58A, 58B within each cluster is limited by the surrounding clusters that are generated. When seeding occurs in depressions, the cluster size is limited by the number library templates 56 that seed in the depression and the size of the depression. In one example, amplification involves cluster generation. In one example of cluster generation, the initially seeded library templates 56 are copied from the hybridized primers by 3′ extension using a high-fidelity DNA polymerase to generate the first amplicons 58A. The original library templates 56 are denatured, leaving the copies immobilized via the primers 42 all around the substrate 12 or 12′. Isothermal bridge amplification, exclusion amplification, or some other form of amplification may be used to amplify the immobilized copies 58A. For example, the copied templates (amplicons 58A) loop over to hybridize to an adjacent, complementary primer, e.g., 40, and a polymerase copies the copied amplicons 58A to form the second amplicons 58B at the 3′ end of the primer 40. The second amplicon 58B is a copy of the first amplicon 58A and thus has the same sequence as the library template 56. The amplicons 58A, 58B form double stranded bridges, which are denatured to form two single stranded strands 58A, 58B. These two amplicons 58A, 58B loop over and hybridize to adjacent, complementary primers 40, 42 and are extended again to form two new double stranded loops. The process is repeated on each template copy by cycles of isothermal denaturation and amplification to create dense clonal clusters of amplicons 58A, 58B. Each cluster of double stranded bridges is denatured, leaving single stranded amplicons 58A, 58B immobilized on the substrate 12 or 12′, as shown in FIG. 5B. This example of clustering is referred to as bridge amplification, and is one example of the amplification that may be performed. It is to be understood that other amplification techniques may be used.

In this example, the amplicons 58B attached to the cleavable second primer 40, 40′ are removed using a cleaving agent for the cleavage site 38′. The cleaving agent that is introduced depends upon the cleavage site 38′ that is to be cleaved. Some cleavage sites 38′ are enzymatically cleavable. For example, 8-oxoguanine can be targeted by Human 8-oxoguanine-DNA glycosylase (hOgg1). For another example, an enzymatically cleavable nucleobase is susceptible to cleavage by reaction with a glycosylase and an endonuclease, or with an exonuclease. One specific example of the cleavable nucleobase is deoxyuracil (dU), which can be targeted by the USER enzyme. Other cleavage sites 38, 38′ are chemically cleavable. As examples, an allyl ether can be cleaved using the reducing agent, such as dithiothreitol (DTT), tris(2-carboxyethyl) phosphine (TCEP), and tris(hydroxypropyl)phosphine (THP); and a vicinal diol (e.g., 1,2-diol) can be cleaved with sodium periodate.

The cleaving agent is introduced into the flow cell 10A or 10C and is allowed to incubate for a time sufficient to cleave the cleavage site 38′. A washing process may then be performed to remove the cleaving agent and the cleaved amplicons 58B. The washing process may involve the introduction of water and a surfactant (e.g., TWEEN® 20 (polyethylene glycol sorbitan monolaurate) from Croda) to the flow cell 10A or 10C. A flow through process may be used for washing.

As the amplicons 58A are attached to the un-cleavable primers 42, they will remain attached after the cleaving agent is removed from the flow cell 10A or 10C in the washing process.

An extension reaction is performed along the first amplicon 58A to generate a first amplicon complement 58A′. This is shown in FIG. 5C.

For the extension reaction, an extension primer is introduced and hybridized, and then a mixture containing nucleotides and a polymerase is introduced into the flow cell 10A or 10C. The extension primer is complementary to the adapter at the 3′ end of the first amplicon 58A. In the example shown in FIG. 5C, the extension primer has the same sequence as the un-cleavable first primer 42, 42′. The nucleotides include the following bases: adenine, cytosine, guanine and thymine. Any polymerase that can accept the nucleotide, and that can successfully incorporate the base of the nucleotide at the 3′ end of the extension primer may be used. Example polymerases include those polymerases from family A, such as Bsu Polymerase, Bst Polymerase, Taq Polymerase, T7 Polymerase, and many others; polymerases from families B and B2, such as Phi29 polymerase and other highly processive polymerases (family B2), Pfu Polymerase (family B), KOD Polymerase (family B), 90N (family B), and many others; polymerases from family C, such as Escherichia coli DNA Pol III, and many others, polymerases from family D, such as Pyrococcus furiosus DNA Pol II, and many others; polymerases from family X, such as DNA Pol μ, DNA Pol β, DNA Pol σ, and many others. The nucleotide mixture may also include a liquid carrier, such as water and/or an ionic salt buffer fluid, e.g., saline citrate at milli-molar to molar concentrations, sodium chloride, potassium chloride, phosphate buffered saline, etc., and other buffers, such as tris(hydroxymethyl)aminomethane (TRIS) or (4-(2-hydroxyethyl)-1-piperazineethanesulfonic acid) (HEPES). The liquid carrier may also include catalytic metal(s) intended for the extension reaction, such as Mg²⁺, Mn²⁺, etc. A single catalytic metal or a combination of catalytic metals may be used, and the total amount may range from about 0.01 mM to about 100 mM.

The temperature of the flow cell 10A or 10C may be adjusted to initiate the template extension reaction. Example temperatures range from about 20° C. to about 70° C. The polymerase enables the extension of the 3′ end of the extension primer using the first amplicon 58A as a template. Because the extension reaction is guided by the first amplicon 58A, the polymerase extension along the first amplicon 58A generates the first amplicon complement 58A′.

FIG. 5D depicts the flow cell 10A after the extension reaction has taken place and after the removable blocking mechanism 30 has been removed. Any of the removable blocking mechanisms 30 described in reference to FIG. 3A through FIG. 3D may be used in this example method, and suitable removal techniques for each of the mechanisms 30 will now be described. It is to be understood that the removal method and the timing of the removal depends upon the type of removable blocking mechanism 30 that is used. As such, while FIG. 5D depicts the first amplicon complement 58A′ formed in the flow cell 10A and the removable blocking mechanism 30 removed from the flow cell 10A, the removable blocking mechanism 30 may be removed before or after the first amplicon complement 58A′ is generated.

In some instances, the removable blocking mechanism 30 includes the plurality of linearizable blocking primers 32, 32′ grafted in a region (e.g., on the substrate 12 as shown in FIG. 5C or in one of the regions A, B) with the primer set 18 or 20 that is to be blocked. In the example shown in FIG. 5A through FIG. 5C, the removable blocking mechanism 30 would include the plurality of linearizable blocking primers 32, 32′ grafted in a region (e.g., on the substrate 12 as shown in FIG. 5C or in one of the regions A, B) with the second primer set 20. In this specific example, one of the plurality of linearizable blocking primers 32, 32′ is respectively hybridized to the cleavable first primer 34, 34′ and the un-cleavable second primer 36, 36′ of the second primer set 20 during amplification; and removal of the removable blocking mechanism 30 (specifically the linearizable blocking primers 32, 32′) involves introducing a cleaving agent to the flow cell 10A or 10C at a temperature that dehybridizes each of the plurality of linearizable blocking primers 32, 32′ from each of the cleavable first primer 34, 34′ and the un-cleavable second primer 36, 36′ of the second primer set 20, whereby the cleaving agent cleaves a cleavage site 38 or 38′ of each of the plurality of linearizable blocking primers 32, 32′.

As described in reference to FIG. 3A, each of the linearizable blocking primers 32, 32′ includes the same cleavage site 38 or 38′ as the cleavable primers 34 or 40 of the primer set 20 or 18 that is not passivated by the removable blocking mechanism 30. In the example shown in FIG. 5C, the linearizable blocking primers 32, 32′ are hybridized to the primer set 20, and the cleavage site of the linearizable blocking primers 32, 32′ is the same as the cleavage site 38′ of the cleavable second primer 40 of the unblocked primer set 18. Thus, the cleaving agent that is used to remove the linearizable blocking primers 32, 32′ also cleaves the second amplicon 58B from the cleavable second primer 40. As such, with this particular blocking mechanism 30, the removal of the second amplicon 58B (described in reference to FIG. 5C) and the removal of the linearizable blocking primers 32, 32′ occurs simultaneously. In this example then, the removable blocking mechanism 30 is removed before the extension reaction takes place to generate the first amplicon complement 58A′.

When the linearizable blocking primers 32, 32′ are used as the removable blocking mechanism 30, cleaving may take place at a temperature that dehybridizes each of the plurality of linearizable blocking primers 32, 32′ from each of the cleavable first primer 34, 34′ and the un-cleavable second primer 36, 36′ of the second primer set 20. The cleaving agent itself may be introduced at this temperature, or the flow cell 10A or 10C may be raised so that the temperature within the channel 16 reaches this temperature. As an example, the temperature ranges from about 70° C. to about 100° C. During cleavage, the linearizable blocking primers 32, 32′ are both cleaved from the substrate 12 or 12′ and are dehybridized from the primers 34, 34′, 36, 36′ of the primer set 20. Alternatively, the linearization of the linearizable blocking primers 32, 32′ may take place at a lower temperature, and then the temperature could be raised to dehybridize each of the plurality of linearizable blocking primers 32, 32′ from each of the cleavable first primer 34, 34′ and the un-cleavable second primer 36, 36′ of the second primer set 20. Dehybridization depends on the length of the oligonucleotides being dehybridized and whether any denaturant (e.g., formamide, NaOH) is added.

In this example, the cleaved and dehybridized linearizable blocking primers 32, 32′ can be removed from the flow cell 10A or 10C with the cleaved second amplicons 58B during the washing process. In this example, the extension reaction described in reference to FIG. 5C then takes place to form the first amplicon complements 58A′.

In other instances, the removable blocking mechanism 30 includes the plurality of blocking primers 44, 44′. In the example shown in FIG. 5A through FIG. 5C, the removable blocking mechanism 30 would include the plurality of blocking primers 44, 44′ respectively hybridized to the cleavable first primer 34, 34′ and the un-cleavable second primer 36, 36′ of the second primer set 20. In this specific example, one of the plurality of blocking primers 44, 44′ is respectively hybridized to the cleavable first primer 34, 34′ and the un-cleavable second primer 36, 36′ of the second primer set 20 during amplification; removal of the removable blocking mechanism 30 (specifically the blocking primers 44, 44′) involves dehybridizing each of the plurality of blocking primers 44, 44′ from the cleavable first primer 34, 34′ and the un-cleavable second primer of the second primer set 30; and the plurality of blocking primers 44, 44′ are washed from the flow cell 10A (or 10C) before cleaving the second amplicon 58B from the cleavable second primer 40, 40′.

When the blocking primers 44, 44′ are used as the removable blocking mechanism 30, removal takes place at a suitable dehybridization temperature. A fluid (e.g., water, or a salt (NAOH), or a buffer) may be introduced at this temperature, or the flow cell 10A or 10C may be raised so that the temperature within the channel 16 reaches this temperature. As an example, the temperature ranges from about 70° C. to about 100° C. At this temperature, the blocking primers 44, 44′ are dehybridized from the primers 34, 34′, 36, 36′ of the primer set 20.

The dehybridized blocking primers 44, 44′ are then removed from the flow cell 10A or 10C using the washing process described herein. In this particular example, removal of the removable blocking mechanism 30 (the blocking primers 44, 44′) takes place after cluster generation is performed with the unblocked primer set, e.g., set 18 in FIG. 5A through FIG. 5F, but before the second amplicon 58B is removed and before the first amplicon complement 58A′ is generated. As such, after the dehybridized blocking primers 44, 44′ are removed from the flow cell 10A, 10C, the method continues with second amplicon 58B removal and the extension reaction, as described in reference to FIG. 5C.

In another example when the blocking primers 44, 44′ are used, the blocking primers 44, 44′ are dehybridized and are present in the flow channel 16 when the cleaving agent is introduced. Once the second amplicons 58B are removed, the blocking primers 44, 44′ and the second amplicons 58B may be removed from the flow channel 16 in a wash process.

In still other instances, the removable blocking mechanism 30 is integrated into linearizable hairpin primers 48, 48′ as described in FIG. 3C. In the example shown in FIG. 5A through FIG. 5C, the removable blocking mechanism 30 would be respectively integrated into the first linearizable hairpin primer 48 with the cleavable first primer 34, 34′ and the second linearizable hairpin primer 38′ with the un-cleavable second primer 36, 36′. In this specific example, the first hairpin formation sequence 46 of the first linearizable hairpin primer 48 would be hybridized to at least a portion of the cleavable first primer 34, 34′ and the second hairpin formation sequence 46′ of the second linearizable hairpin primer 48′ would be hybridized to at least a portion of the un-cleavable second primer 36, 36′ during the amplification; and removal of the removable blocking mechanism 30 would involve introducing a cleaving agent of the first linearizable hairpin primer 48 and the second linearizable hairpin primer 48′.

As described in reference to FIG. 3C, each of the linearizable hairpin primers 48, 48′ includes the cleavage site 38″, which is orthogonal to the cleavage site 38 or 38′ of the cleavable primer 34 or 40 that is passivated. In the example of FIG. 5A through FIG. 50, the cleavage site 38″ is orthogonal to the cleavage site 38 of the cleavable primer 34, 34′ (which is passivated), and may be the same as or different than the cleavage site 38′ of the unblocked cleavable primer 40, 40′.

When the cleavage sites 38″, 38′ are the same, the removal of the second amplicon 58B and the removal of the linearizable hairpin primers 48, 48′ will take place simultaneously upon exposure to a single cleaving agent. In this example, the cleaving agent of the first linearizable hairpin primer 48 and the second linearizable hairpin primer 48′ also cleaves the second amplicon 58B. The single cleaving agent is introduced into the flow cell 10A (or 10C) at a temperature that is sufficient to dehybridize each of the hairpin portions 50, 50′ from each of the cleavable first primer 34, 34′ and the un-cleavable second primer 36, 36′ of the second primer set 20. The cleaving agent itself may be introduced at this temperature, or the flow cell 10A or 10C may be raised so that the temperature within the channel 16 reaches this temperature. As an example, the temperature ranges from about 70° C. to about 100° C. During removal, the hairpin portions 50, 50′ are both cleaved and are dehybridized from the primers 34, 34′, 36, 36′ of the primer set 20. In this example, the cleaved and dehybridized hairpin portions 50, 50′ can be removed from the flow cell 10A or 10C with the cleaved second amplicons 58B during the washing process. In this example then, the removable blocking mechanism 30 is removed before the extension reaction takes place to generate the first amplicon complement 58A′. The extension reaction described in reference to FIG. 5C then takes place to form the first amplicon complements 58A′.

When the cleavage sites 38″, 38′ are different, the removal of the second amplicon 58B and the removal of the linearizable hairpin primers 48, 48′ will take place sequentially upon exposure to two different cleaving agents. The cleaving agent for the cleavage site 38″ may be introduced into the flow cell 10A or 10C before or after the cleaving agent for the cleavage site 38′, and thus the linearizable hairpin primers 48, 48′ may be removed before or after the second amplicons 58B are removed. In one example, the cleaving agent for the cleavage site 38″ is introduced first to dehybridize and cleave the linearizable hairpin primers 48, 48′, the dehybridized and cleaved linearizable hairpin primers 48, 48′ are removed from the flow cell 10A or 10C, and then the cleaving agent for the cleavage site 38′ is introduced into the flow cell 10A or 10C to cleave the second amplicons 58B. In another example, the cleaving agent for the cleavage site 38′ is introduced first into the flow cell 10A or 10C to cleave the second amplicons 58B, the cleaved second amplicons 58B are removed from the flow cell 10A or 10C, and then the cleaving agent for the cleavage site 38″ is introduced first to dehybridize and cleave the linearizable hairpin primers 48, 48′.

In any of these examples, at least the cleaving agent for the site 38″ is added at a temperature that is sufficient to dehybridize each of the plurality of hairpin portions 50, 50′ from each of the cleavable first primer 34, 34′ and the un-cleavable second primer 36, 36′ of the second primer set 20. When exposed to the cleaving agent for the site 38″, the hairpin portions 50, 50′ are both cleaved and dehybridized from the primers 34, 34′, 36, 36′ of the primer set 20.

Regardless of the order in which the linearizable hairpin primers 48, 48′ and the amplicons 58B are removed, both processes occur before the extension reaction takes place to generate the first amplicon complement 58A′. The extension reaction described in reference to FIG. 5C then takes place to form the first amplicon complements 58A′.

In still other instances, the removable blocking mechanism 30 is the hydrophobic barrier layer 52. In the example shown in FIG. 5A through FIG. 5C, the hydrophobic barrier layer 52 overlies the second primer set 20; and removal of the removable blocking mechanism 30 involves washing the hydrophobic barrier layer from the flow cell 10A or 10C before cleaving the second amplicon 58B from the cleavable second primer 40.

When the hydrophobic barrier layer 52 is used as the removable blocking mechanism 30, removal takes place by introducing a reagent that dissolves the hydrophobic barrier layer 52. Example reagents include organic solvents (e.g., toluene, dimethylformamide, methylethylketone, ethanol, etc.) or other developing solutions, such as a base (NaOH, KOH, trimethylamine, DiPEA, NH₄OH, etc.).

The dissolved hydrophobic barrier layer 52 is then removed from the flow cell 10A or 10C using the washing process described herein. In this particular example, removal of the removable blocking mechanism 30 (the hydrophobic barrier layer 52) may take place after cluster generation is performed with the unblocked primer set, e.g., set 18 in FIG. 5A through FIG. 5F, before or after the second amplicon 58B is removed, and before the first amplicon complement 58A′ is generated. As such, in some instances, after the second amplicons 58B and the dissolved hydrophobic barrier layer 52 are removed from the flow cell 10A, 10C, the method continues with the extension reaction, as described in reference to FIG. 5C. In other instances, after the dissolved hydrophobic barrier layer 52 is removed from the flow cell 10A, 10C, the method continues with removal of the second amplicons 58B, followed by the extension reaction, as described in reference to FIG. 5C.

After the removable blocking mechanism 30 is removed and the first amplicon complement 58A′ is generated, the method described in FIG. 5A through FIG. 5F continues with dehybridizing the first amplicon complement 58A′ from the first amplicon 58A (FIG. 5E); and initiating seeding of the first amplicon complement 58A′ to one of the primers, e.g., cleavable first primer 34, 34′ of the second primer set 20 (FIG. 5E). Dehybridization of the first amplicon complement 58A′ is performed using a suitable dehybridization temperature, and may be performed in the presence of a suitable liquid (e.g., a denaturant). Thermal cycling is used to initiate seeding of the first amplicon complement 58A′ to one of the primers, e.g., cleavable first primer 34, 34′ of the second primer set 20 (FIG. 5E). During thermal cycling, the temperature may be reduced from the dehybridization temperature to a temperature suitable for hybridization. At least some of the released first amplicon complements 58A′ will diffuse through the channel 16 toward the now unblocked primer set, e.g., set 20, and will respectively seed to one of the primers, e.g., cleavable first primer 34, 34′ of the second primer set 20 (FIG. 5E).

The first amplicon complements 58A′ will randomly seed on the substrate, e.g., 12 or 12′, or in the region, e.g., A or B opposed to the substrate 12′ or 12 or region B or A on which they were generated. During analysis of the sequencing data, the UMI or UDI may be used to bioinformatically link the amplicons 58A, 58D generated from the same library template 56.

By reducing the channel 16 height/depth of the flow cell 10A, the likelihood of first amplicon complements 58A′ seeding at a spatially co-located x-y region of the opposed substrate, e.g., substrate 12, will increase, as the diffusion distance is reduced. This may be due to the fact that drifting of the first amplicon complements 58A′ in the x and/or y directions is reduced when the channel distance is smaller.

The likelihood of the first amplicon complements 58A′ seeding at a spatially co-located x-y region of the opposed substrate 12 may be further increased using electrophoresis. In this example, the flow cell 10A includes individually addressable electrodes 60A, 60B, 60C embedded in the substrate 12, as shown in hidden line in FIG. 5D. After the dehybridization of the first amplicon complements 58A′, the method further includes addressing an electrode 60A, 60B and/or 60C of the flow cell 10A that is spatially co-located with the second primer set 20, thereby electrophoretically attracting the first amplicon complements 58A to the primers 34, 34′, 36, 36′ overlying the electrodes 60A, 60B and/or 60C. Examples of suitable electrode materials include any electrode materials that are transparent to the optical wavelengths used in sequencing. One example electrode material is indium tin oxide (ITO).

Referring now to FIG. 5F, amplification of the seeded first amplicon complements 58A′ may be performed in the same manner as the amplification of the initially seeded library template 56. This amplification process generates a third amplicon 58C attached to the cleavable first primer 34, 34′ and a fourth amplicon 58D attached to the un-cleavable second primer 36, 36′.

The method then includes cleaving the third amplicon 58C from the cleavable second primer 36, 36′; and initiating a sequencing operation of the first amplicon 58A and the fourth amplicon 58D.

In this example, the amplicons 58C attached to the cleavable first primer 34, 34′ are removed using a cleaving agent for the cleavage site 38. The cleaving agent that is introduced depends upon the cleavage site 38 that is to be cleaved. Any of the examples disclosed herein may be used. The cleaving agent is introduced into the flow cell 10A or 10C and is allowed to incubate for a time sufficient to cleave the cleavage site 38. The washing process may then be performed to remove the cleaving agent and the cleaved amplicons 58C.

As the amplicons 58D are attached to the un-cleavable primers 36, 36′, they will remain attached after the cleaving agent is removed from the flow cell 10A or 10C in the washing process.

Due to the sequences and orthogonal cleavage sites 38, 38′ of the primer sets 20, 18, the first amplicon 58A and the fourth amplicon 58D are opposite strands (forward and reverse strands, or vice versa) of the initially introduced library templates 56. This enables simultaneous paired-end sequencing, which can be performed using a sequencing-by-synthesis method.

It is to be understood that sequencing primers for initiating the first and second reads of the DNA molecules (e.g., Read 1 and Read 2 primers) and for initiating reads of the UMI or for initiating reads of the respective indices of a UDI (e.g., index primers) may be introduced prior to introducing an incorporation mix. These primers may be introduced in any order and render the various regions of the amplicons 58A, 58D ready for sequencing. In the examples disclosed herein, the Read 1 primers bind to the sequencing binding site of the forward amplicons, e.g., 58D, while the Read 2 primers bind to the sequencing binding site of the reverse amplicons, e.g., 58A. This enables the simultaneous sequencing.

The incorporation mix may include a plurality of fully functional nucleotides, the polymerase, and a liquid carrier. The liquid carrier of the incorporation mix may be water and/or an ionic salt buffer fluid, such as saline citrate at milli-molar to molar concentrations, sodium chloride, potassium chloride, phosphate buffered saline, etc., and other buffers, such as tris(hydroxymethyl)aminomethane (TRIS) or (4-(2-hydroxyethyl)-1-piperazineethanesulfonic acid) (HEPES). The liquid carrier may also include catalytic metal(s) intended for the incorporation reaction, such as Mg²⁺, Mn²⁺, Ca²⁺, etc. A single catalytic metal or a combination of catalytic metals may be used, and the total amount may range from about 0.01 mM to about 100 mM.

The fully functional nucleotide (FFN) includes the nucleotide, a 3′ OH blocking group attached to the sugar of the nucleotide, and a fluorophore attached to the base of the nucleotide. The nucleotide of the FFN may be any nucleotide describe herein.

The nucleotide of the FFN also includes a 3′ OH blocking group attached thereto. The 3′ OH blocking group may be linked to the 3′ oxygen atom of the sugar molecule in the nucleotide. The 3′ OH blocking group may be a reversible terminator that allows only a single-base incorporation to occur in each sequencing cycle. The reversible terminator stops additional bases from being incorporated into a nascent strand that is complementary to the amplicon. This enables the detection and identification of a single incorporated base into each of the amplicons 58A, 58D. The 3′ OH blocking group can subsequently be removed, enabling additional sequencing cycles to take place at each amplicon 58A, 58D. Examples of different 3′ OH blocking groups include a 3′-ONH₂ reversible terminator, a 3′-O-allyl reversible terminator (i.e., —CH═CHCH₂), and 3′-O-azidomethyl reversible terminator (i.e., —CH₂N₃). Other suitable reversible terminators include o-nitrobenzyl ethers, alkyl o-nitrobenzyl carbonate, ester moieties, other allyl-moieties, acetals (e.g., tert-butoxy-ethoxy), MOM (—CH₂OCH₃) moieties, 2,4-dinitrobenzene sulfenyl, tetrahydrofuranyl ether, 3′ phosphate, ethers, —F, —H₂, —OCH₃, —N₃, —HCOCH₃, and 2-nitrobenzene carbonate.

The nucleotide of the FFN also includes a fluorophore attached to the base of the nucleotide. The fluorophore may be any optically detectable moiety, including luminescent, chemiluminescent, fluorescent, fluorogenic, chromophoric and/or chromogenic moieties. Some examples of suitable optically detectable moieties include fluorescein labels, rhodamine labels, cyanine labels (e.g., Cy3, Cy5, and the like), and the ALEXA® family of fluorescent dyes and other fluorescent and fluorogenic dyes. The fluorophore may be attached to the base of the nucleotide using any suitable linker molecule. In an example, the linker molecule is a spacer group of formula —((CH₂)₂O)ₙ— wherein n is an integer between 2 and 50.

In one example, the incorporation mix includes a mixture of different FFNs, which include different bases, e.g., A, T, G, C (as well as U or I). It may also be desirable to utilize a different type of fluorophore for the different FFNs. For example, the fluorophores may be selected so that each fluorophore absorbs excitation radiation and/or emits fluorescence, at a distinguishable wavelength from the other fluorophores. Such distinguishable analogs provide an ability to monitor the presence of different fluorophores simultaneously in the same reaction mixture. In some examples, one of the four FFNs in the incorporation mix may include no fluorophore, while the other three labeled FFNs include different fluorophore.

Any polymerase that can accept the fully functional nucleotide, and that can successfully incorporate the base of the fully functional nucleotide into a nascent strand along the amplicons 58A, 58D may be used. Examples polymerases include those polymerases from family A, such as Bsu Polymerase, Bst Polymerase, Taq Polymerase, T7 Polymerase, and many others; polymerases from families B and B2, such as Phi29 polymerase and other highly processive polymerases (family B2), Pfu Polymerase (family B), KOD Polymerase (family B), 90N (family B), and many others; polymerases from family C, such as Escherichia coli DNA Pol III, and many others, polymerases from family D, such as Pyrococcus furiosus DNA Pol II, and many others; polymerases from family X, such as DNA Pol μ, DNA Pol β, DNA Pol σ, and many others.

In this example method, any example of the incorporation mix is introduced into the flow cell 10A or 10C, e.g., via the inlet. When the incorporation mix is introduced into the flow cell 10A or 10C, the mix enters the flow channel 16, and contacts the surface chemistry where the amplicons 58A, 58D are present.

The incorporation mix is allowed to incubate in the flow cell 10A or 10C, and FFNs are incorporated by polymerases into nascent strands respectively generated along the amplicons 58A, 58D. During incorporation, one of FFNs is incorporated, by a respective polymerase, into one nascent strand that extends one sequencing primer and that is complementary to one of the amplicons 58A, 58D. Incorporation is performed in a template strand dependent fashion, and thus detection of the order and type of FFNs added to the nascent strands can be used to determine the sequence of the amplicons 58A, 58D. Incorporation occurs in at least some of the amplicons 58A, 58D across the substrates 12, 12′ or regions A, B during a single sequencing cycle. As such, in at least some of the amplicons 58A, 58D across the flow cell 10A or 10C, respective polymerases extend the hybridized sequencing primers by one of the FFNs in the incorporation mix.

The incorporated FFNs include the reversible termination property due to the presence of the 3′ OH blocking group, which terminates further sequencing primer extension on the nascent strand once the FFN has been added.

After a desired time for incubation and incorporation, the incorporation mix, including at least some non-incorporated FFNs, may be removed from the flow cell 10A or 10C during a wash cycle. The wash cycle may involve a flow-through technique, where a washing solution (e.g., buffer) is directed into, through, and then out of flow channel 16, e.g., by a pump or other suitable mechanism.

Without further incorporation taking place, the most recently incorporated FFNs can be detected through an imaging event. During the imaging event, an illumination system (not shown) may provide an excitation light to the flow cell surfaces containing the surface chemistry. The fluorophore of the incorporated FFNs emit optical signals in response to the excitation light. The most recently incorporate FFNs in the nascent strands of the amplicons 58A, 58C can be detected simultaneously.

After imaging is performed, a cleavage mix may then be introduced into the flow cell 10A or 10C. In this example, the cleavage mix is capable of i) removing the 3′ OH blocking group from the incorporated FFNs, and ii) cleaving the fluorophore from the FFNs. Examples of 3′ OH blocking groups and suitable de-blocking agents/components in the cleavage mix may include: ester moieties that can be removed by base hydrolysis; allyl-moieties that can be removed with Nal, chlorotrimethylsilane and Na₂S₂O₃ or with Hg (II) in acetone/water; azidomethyl which can be cleaved with phosphines, such as tris(2-carboxyethyl) phosphine (TCEP) or tri (hydroxypropyl)phosphine (THP); acetals, such as tert-butoxy-ethoxy which can be cleaved with acidic conditions; MOM (—CH₂OCH₃) moieties that can be cleaved with LiBF₄ and CH₃CN/H₂O; 2,4-dinitrobenzene sulfenyl which can be cleaved with nucleophiles such as thiophenol and thiosulfate; tetrahydrofuranyl ether which can be cleaved with Ag(I) or Hg(II); and 3′ phosphate which can be cleaved by phosphatase enzymes (e.g., polynucleotide kinase). Examples of suitable fluorophore cleaving agents/components in the cleavage mix may include: sodium periodate, which can cleave a vicinal diol; phosphines, such as tris(2-carboxyethyl) phosphine (TCEP) or tri (hydroxypropyl)phosphine (THP), which can cleave azidomethyl linkages; palladium and THP, which can cleave an allyl; bases, which can cleave ester moieties; or any other suitable cleaving agent.

Wash(es) may take place between the various fluid delivery steps. The sequencing cycle can then be repeated n times to extend the sequencing primers by n nucleotides, thereby detecting a sequence of length n.

After sequencing, the method may further include linking reads of the first amplicon 58A with reads of the fourth amplicon 58D by the unique molecular index (UMI) that is part of the library template 56 (from which the amplicons 58A, 58D are generated). Alternatively, the method may further include linking reads of the first amplicon 58A with reads of the fourth amplicon 58D by two indexes (part of a UDI) that are part of the library template 56 (from which the amplicons 58A, 58D are generated).

In the method of FIG. 5A through FIG. 5F, the concentration of the dehybridized amplicon complements that seed to the primer(s) 34, 34′, 36, 36′ of the second primer set 20 may be controlled. In one example, this involves initially grafting the plurality of the first primer set 18 such that some of the cleavable second primers 40, 40′ of the plurality of the first primer set 18 include a first cleavage site 38′ and some other of the cleavable second primers 40, 40′ of the plurality of the first primer set 18 include a second cleavage site, e.g., cleavage site 38, that is orthogonal to the first cleavage site 38′. As such, in this example, some of the cleavable second primers 40, 40′ have the same cleavage site 38 as the cleavable first primers 34, 34′, while some other of the cleavable second primers 40, 40′ have a different cleavage site 38′ than the cleavable first primers 34, 34′. The number ratio of the primers 40, 40′ with the cleavage site 38 to the primers 40, 40′ with the cleavage site 38′ may be controlled so that a desired percentage of the second amplicons 58B remain after the cleaving agent for the first cleavage site 38′ is introduced (e.g., as described in reference to FIG. 5B). In this example, the first amplicons 58A and a predetermined percentage of the second amplicons 58B will remain attached to the substrate 12′, and thus will be available for the extension reaction. In this example, two extension primers are used which generate amplicon complements for both the amplicons 58A and 58B. Both types of amplicon complements are subsequently dehybridized from the substrate 12′ and hybridized to the substrate 12. Thus, by controlling the ratio of the cleavage sites 38: 38′ used in the first round of amplification, one can control the number of amplicon complements, e.g., 58A′ and 58B′, that are generated and available for subsequent dehybridization and re-hybridization.

In another example, the concentration of amplicon complements 58A′ can be controlled by titrating a concentration of the extension primer used during the extension reaction. If the concentration of the extension primers is lower, a lower number of amplicon complements 58A′ will be generated, and thus available for dehybridization and re-hybridization.

In another example method, after clustering is performed on the substrate 12′, the blocking mechanism 30 could be dehybridized and removed from the flow cell 10A or 10C, and then the amplicons 58B could be cleaved and seeded to the primer 34 or 36 on the substrate 12 or region B. In this example, extension of the amplicon 58A on the substrate 12 or region B would not take place.

Referring now to the example method depicted in FIG. 6A through FIG. 6D, the flow cell 10B or 10D can be utilized. In the example shown in FIG. 6A through FIG. 6D, the flow cell 10B is depicted. Also in the example shown in FIG. 6A through FIG. 6D, the first primer set 18 includes the un-cleavable first primer 42, 42′ and the cleavable second primer 40, 40′; the second primer set 20 includes the cleavable first primer 34, 34′ and the un-cleavable second primer 36, 36′; and each of the cleavable first primer 34, 34′ and the cleavable second primer 40, 40′ is blocked with a removable blocking mechanism 30. It is to be understood that in this example, the examples of the removable blocking mechanism 30 described in reference to FIG. 4A and FIG. 4B may be used. It is to be further understood that for ease of illustration, the additional reference numbers, e.g., 34′, 36′, 40′, 42′, for the primer sets 20B, 18B are not shown in FIG. 6A through FIG. 6D, but these primers 34′, 36′, 40′, 42′ could be used.

This method involves introducing complementary library templates 56, 56′ into the flow cell 10B or 10D (which includes spatially separated first and second primer sets 18, 20 as described herein), whereby the complementary library template strands 56, 56′ respectively seed to the un-cleavable first primer 42, 42′ and the un-cleavable second primer 36, 36′ (FIG. 6A); performing a strand extension reaction along the seeded complementary library template strands 56, 56′, thereby generating a first amplicon 58A attached to the un-cleavable first primer 42, 42′ and a second amplicon 58B attached to the un-cleavable second primer 36, 36′ (FIG. 6B); dehybridizing the complementary library template strands 56, 56′ from each of the first amplicon 58A and the second amplicon 58B (FIG. 6C); removing the removable blocking mechanism 30 from each of the cleavable first primer 34, 34′ and from the cleavable second primer 40, 40′ (FIG. 6C); and initiating amplification of the first amplicon 58A using the first primer set 18 and the second amplicon 58B using the second primer set 20 (FIG. 6D). FIG. 6D depicts the amplicons 58A, 58C and 58B, 58D generated after simultaneous amplification.

In this example method, the library templates 56 may be generated as described herein in reference to FIG. 5A. The complementary library template strands 56′ may be generated by exposing the original library template strands 56 to a polymerase chain reaction (PCR). PCR is performed off-board the flow cell 10B or 10D, and is used to generate an amplified library. The amplified library will increase the likelihood that both strands (e.g., the original library template 56 and its complement 56′) are seeded.

The library templates 56, 56′ are then introduced into the flow cell 10B or 10D. While one of each of the library templates 56, 56′ is shown, it is to be understood that a plurality of different library templates 56 (e.g., each generated from a single DNA sample) and their complements 56′ may be introduced into the flow cell 10B or 10D together.

In this example, the cleavable first and second primers 34, 34′ and 40, 40′ are blocked, leaving the un-cleavable second and first primers 36, 36′ and 42, 42′ free to hybridize to the library templates 56, 56′ introduced into the flow cell 10B or 10D. In the example shown in FIG. 6A, the adapters incorporated into opposed ends of each DNA molecule to form the library templates 56 respectively include the complementary sequence of the un-cleavable first primer 42, 42′ (e.g., P5′ without the cleavage site) and the same sequence as the un-cleavable second primer 36, 36′ (e.g., P7 without the cleavage site). As such, the library templates 56 respectively hybridize to the unblocked un-cleavable first primers 42, 42′ at the surface of the substrate 12′ (or in region A of the flow cell 10D). It follows that the adapters of each of the complementary library templates 56′ include the same sequence as the un-cleavable first primer 42, 42′ (e.g., P5 without the cleavage site) and the complementary sequence of the un-cleavable second primer 36, 36′ (e.g., P7′ without the cleavage site). As such, the complementary library templates 56′ respectively hybridize to the unblocked un-cleavable second primers 36, 36′ at the surface of the substrate 12 (or in region B of the flow cell 10D).

In FIG. 6B, an extension reaction is performed from the 3′ end of each of the un-cleavable second and first primers 36, 36′ and 42, 42′ using the respectively hybridized library templates 56′, 56 as the template strand. This extension reaction utilizes the 3′ end of the un-cleavable second and first primers 36, 36′ and 42, 42′ primers as the starting point for the extension reaction, and thus additional extension primers are not introduced in this example. For this extension reaction, a mixture containing nucleotides and a polymerase is introduced into the flow cell 10B or 10D, and the temperature of the flow cell 10B or 10D may be adjusted to initiate the template extension reaction. The polymerase enables the extension of the 3′ end of the un-cleavable second and first primers 36, 36′ and 42, 42′ using the hybridized library templates 56′, 56 as the templates. As such, the polymerase extension generates the first amplicon 58A along the library template 56 and the second amplicon 58B along the complementary library template 56′. As shown in FIG. 6B, the first amplicon 58A is immobilized on the substrate 12′ through the un-cleavable first primer 42, 42′ and the second amplicon 58B is immobilized on the substrate 12 through the un-cleavable second primers 36, 36′. While one of each amplicon 58A, 58B is shown in FIG. 6B, it is to be understood that amplicons 58A, 58B may be generated wherever library templates 56, 56′ are hybridized. The blocking mechanism 30 on the cleavable first and second primers 34, 34′ and 40, 40′ prevents extension at these primers 34, 34′ and 40, 40′.

FIG. 6C depicts the flow cell 10B (or 10D) after the library templates 56, 56′ and the removable blocking mechanism 30 are removed. Removal of these components 56, 56′ and 30 may take place in any order.

Removal of the library templates 56, 56′ takes place at a suitable dehybridization temperature. A fluid (e.g., water or a salt or formamide) may be introduced at this temperature, or the temperature of the flow cell 10B or 10D may be raised so that the temperature within the channel 16 reaches this temperature. As an example, the temperature ranges from about 70° C. to about 100° C. At this temperature, the library templates 56, 56′ are dehybridized from the amplicons 58A, 58B and can be washed from the flow cell 10B or 10D.

Removal of the removable blocking mechanism 30 will depend upon the type of blocking mechanism 30 that is used. In one example, the removable blocking mechanism 30 is the terminal phosphate group 54 positioned at the 3′ end of each of the cleavable first primer 34, 34′ and the cleavable second primer 40, 40′. The terminal phosphate group 54 may be removed by introducing an enzyme with 3′-phosphatase activity (e.g., T4 phage polynucleotide kinase (PNK) or alkaline phosphatase (AP or ALP)). These enzymes process the phosphate ends, converting them into 3′-hydroxyls and render them ready for primer alteration (e.g., amplification).

In another example, the removable blocking mechanism 30 is respectively integrated into the first linearizable hairpin primer 48″ with the cleavable first primer 34 and the second linearizable hairpin primer 48″ with the cleavable second primer 40 (as described in reference to FIG. 4C). During the strand extension reaction described in reference to FIG. 6B, the first hairpin formation sequence 46″ of the first linearizable hairpin primer 50″ is hybridized to at least a portion of the cleavable first primer 34, 34′ and the second hairpin formation sequence 46″ of the second linearizable hairpin primer 50″ is hybridized to at least a portion of the cleavable second primer 40, 40′. Removal of this particular example of the removable blocking mechanism 30, 48″, 48″ involves introducing a cleaving agent of the first linearizable hairpin primer 48″, 48″ and the second linearizable hairpin primer 48″, 48″′.

The cleaving agent used will depend upon the cleavage site 38″ of the hairpin primers 48″, 48″. This cleavage site 38″ is orthogonal to the cleavage sites 38, 38′ of each of the cleavable first primer 34, 34′ and the cleavable second primer 40, 40′. The cleaving agent is introduced into the flow cell 10B or 10D and is allowed to incubate for a time sufficient to cleave the cleavage site 38″. The temperature for this cleaving reaction is also sufficient to dehybridize each of the hairpin portions 50″, 50′″ from each of the cleavable first and second primers 34, 34′ and 40, 40′. As such, in this example, the hairpin portions 50″, 50″ are both cleaved and dehybridized from the cleavable first and second primers 34, 34′ and 40, 40′. In this example, the cleaved and dehybridized hairpin portions 50″, 50″ can be removed from the flow cell 10B or 10D during a washing process.

After the library templates 56, 56′ and the removable blocking mechanism 30 are removed, the amplicons 58A, 58B may then be amplified simultaneously. Amplification is initiated to form clusters of the amplicons 58A, 58B at the surfaces of the respective substrates 12′, 12. Each cluster formed on the substrate 12′ includes amplicons 58A, 58C that are respectively attached to the primers 42, 42′ and 40, 40′. Each cluster formed on the substrate 12 includes amplicons 58B, 58D that are respectively attached to the primers 36, 36′ and 34, 34′. Bridge amplification is one example of the amplification that may be performed. It is to be understood that other amplification techniques may be used.

While not shown in the FIG. 6 series, the amplicons 58C and 58D attached to the cleavable second primer 40, 40′ and the cleavable first primer 34, 34′ are removed using cleaving agents for the cleavage site 38′, 38. The cleaving agents are introduced sequentially and will depend upon the cleavage sites 38′, 38 that are to be cleaved. Each cleaving agent is allowed to incubate for a time sufficient to cleave the cleavage site 38′ or 38. A washing process as described herein may then be performed to remove the cleaving agent and the cleaved amplicons 58B. As the plurality of amplicons 58A, 58B are attached to the un-cleavable primers 42, 42′ and 36, 36′, they will remain attached after the cleaving agents are removed from the flow cell 10A or 10C in the washing process.

The plurality of amplicons 58A, 58B may then be simultaneously sequenced as described in reference to the FIG. 5 series.

In still other example methods, rolling circle amplification is used to generate the library template 56.

In one example, rolling circle amplification is performed off-board the flow cell 10G. In this example, the DNA nucleic acid sample is fragmented, and each library template 56 is incorporated into a circular template and exposed to linear rolling circle amplification (RCA). As a result of RCA, a longer single stranded molecule is formed, which includes several amplicons of the original library template 56. This product is referred to the RCA product.

The RCA products are then introduced into the flow cell 10G, where they electrostatically seed to the polymeric hydrogel 28 (without the capture primers 41) that is not covered by the removable blocking mechanism 30 (i.e., the hydrophobic barrier layer 52). The seeded RCA products are then primed with a non-saturating concentration of an extension primer, followed by an extension reaction, to generate copies of a portion of the RCA product (representing multiple repeats of the DNA molecule and the sequencing primer binding sites). The extension reaction may be initiated by introducing the mixture containing nucleotides and a polymerase. The copy(ies) of the RCA product will be the reverse complements of the original strand.

The blocking mechanism 30, 52 is then removed using any of the methods described herein for the hydrophobic barrier layer 52. The removed blocking mechanism 30 is washed from the flow cell 10G.

The copy(ies) of the RCA product are then dehybridized by increasing the temperature to a suitable dehybridization temperature as described herein. The copy(ies) of the RCA products electrostatically seed on the now exposed polymeric hydrogel 28′.

The RCA products and reverse complements may then be simultaneously sequenced as described in reference to the FIG. 5 series. Due to the likely shorter length of the copied RCA product (i.e., the reverse complements on the other substrate 12 or 12′), the fluorescence intensity of the clusters during sequencing on the other substrate 12 or 12′ may be dimmer than the original RCA products on the substrate 12′ or 12. To compensate for this potential lower intensity, the illumination intensity for the other substrate 12 or 12′ may be adjusted, or changes to the relative Read 1 and Read 2 primer concentrations may be adjusted.

In another example, rolling circle amplification is performed on-board the flow cell 10G. In this example, a circular template (including a library template 56 from a longer DNA sample) is first seeded to the capture primers 41 (which are included in this example). The removable circular template is prevented from sticking to the polymeric hydrogel 28′ by the removable blocking mechanism 30, 52. Rolling circle amplification is then performed (e.g., with a primer, nucleotides, and polymerase) on board the flow cell 10G. After the circular template is amplified, the reaction components are removed from the flow cell 10G.

The generated RCA products are then primed with a non-saturating concentration of an extension primer, followed by an extension reaction, to generate the copies.

The blocking mechanism 30, 52 is then removed from polymeric hydrogel 28′ as described herein. The removed blocking mechanism 30 is washed from the flow cell 10G.

The copy(ies) of the RCA product are dehybridized by increasing the temperature to a suitable dehybridization temperature as described herein. The copy(ies) of the RCA product are then dehybridized by increasing the temperature to a suitable dehybridization temperature as described herein. The copy(ies) of the RCA products electrostatically seed on the now exposed polymeric hydrogel 28′.

The RCA products and reverse complements may then be simultaneously sequenced as described in reference to the FIG. 5 series.

Flow Cells with One Sided Surface Chemistry

As depicted in FIG. 7, another example of the flow cell 10E includes one of the primer sets 18 or 20 immobilized on one of the substrates 12 as described in reference to the flow cells 10A and 10C, and the other substrate 12″ includes the polymeric hydrogel 28″ in the lane 14″, but does not have the other of the primer sets 20 or 18 immobilized thereon. The other primer set 20 or 18 is introduced after the library templates 56 are introduced and seeded on the one substrate 12. As such, the other primer set 20 or 18 is part of a separate grafting fluid that may be included in a kit with the flow cell 10E.

In this example, the substrate 12, polymeric hydrogel 28, and immobilized primer set 18 or 20 may be any of the examples described herein in reference to the flow cells 10A and 10C. Additionally, the other substrate 12″ and the polymeric hydrogel 28″ may be any of the examples described herein for the substrate 12′ and the polymeric hydrogel 28′. The substrate 12 and the other substrate 12″ may be bonded as described herein.

The grafting fluid includes the other of the primer sets 20 or 18 that is not bonded to the substrate 12 in the flow cell 10E. The primers, e.g., 34, 36, or 34′, 36′, or 40, 42, or 40′, 42′ have a 5′ terminal end group that is capable of attaching to the polymeric hydrogel 28″ already present in the flow cell 10E. Any of the examples set forth herein may be used. In another example, the polymeric hydrogel 28″ may be biotinylated. In this example, the polymeric hydrogel 28″ is functionalized with biotin surface groups and the primers, e.g., 34, 36, or 34′, 36′, or 40, 42, or 40′, 42′, include biotin as the 5′ end group. Additional streptavidin, avidin, neutravidin, or the like is added or pre-attached to the biotinylated polymeric hydrogel 28″ or to primer biotin groups to indirectly attach the biotin groups (of the hydrogel 28″ and primers) to one another.

The primer fluid includes a carrier fluid and the primers, e.g., 34, 36, or 34′, 36′, or 40, 42, or 40′, 42′ of the primer set 20 or 18 that is not bonded to the substrate 12 in the flow cell 10E. The carrier fluid may be water and/or an ionic salt buffer.

One specific example of the kit includes the flow cell 10E, which includes the first substrate 12, the second substrate 12″ attached to the first substrate 12 (wherein the second substrate 12″ is free of primers), a flow channel 16 defined between the first substrate 12 and the second substrate 12″, and the first primer set 18 attached to the first substrate 12, the first primer set 18 including an un-cleavable first primer 40, 40′ and a cleavable second primer 42, 42′; and the grafting mix, which includes the liquid carrier, and the second primer set 20 to be attached to the second substrate 12″ of the flow cell 10E (e.g., through the polymeric hydrogel 28″), the second primer set 20 including the cleavable first primer 34, 34′ and the un-cleavable second primer 36, 36′.

Method for Using Flow Cells with One Sided Surface Chemistry

The flow cell 10E shown in FIG. 7 may be used in the method that is shown schematically in FIG. 8A through FIG. 8F. In the example shown in FIG. 8A through FIG. 8F, the first primer set 18 includes the un-cleavable first primer 42, 42′ and the cleavable second primer 40, 40′ and the second primer set 20 includes the cleavable first primer 34, 34′ and the un-cleavable second primer 36, 36′. The method may alternatively be performed where the primers 34, 34′, 36, 36′ of the second primer set 20 are present in the flow cell 10E at the outset and the primer set 18 is introduced during the method. For ease of illustration, it is to be understood that the additional reference numbers, e.g., 34′, 36′, 40′, 42′, for the primer sets 20B, 18B are not shown in FIG. 8A through FIG. 8F, but these primers 34′, 36′, 40′, 42′ could be used.

This example method includes introducing a library template 56 into the flow cell 10E, whereby the library template 56 seeds to one of the primers 40, 40′, 42, 42′ of the first primer set 18 (FIG. 8A); initiating amplification of the library template 56 using the first primer set 18, thereby generating a first amplicon 58A attached to the un-cleavable first primer 42 and a second amplicon 58B attached to the cleavable second primer 40 (FIG. 8C); either after i) the library template 56 is seeded (FIG. 8B) or ii) the library template 56 is amplified (FIG. 8C), introducing a second primer set 20 to the second substrate 12″ (shown in FIG. 8B and FIG. 8C); cleaving the second amplicon 58B from the cleavable second primer 40 (FIG. 8D); performing an extension reaction along the first amplicon 58A to generate a first amplicon complement 58A′ (FIG. 8D); dehybridizing the first amplicon complement 58A′ from the first amplicon 58A (FIG. 8E); and initiating seeding of the first amplicon complement 58A′ to one of the primers 34, 36 of the second primer set 20 (FIG. 8E). FIG. 8F depicts the amplicons 58C, 58D generated from the seeded and amplified first amplicon complements 58A′.

In FIG. 8A, the library template 56 is introduced into the flow cell 10E. While a single library template 56 is shown, it is to be understood that a plurality of different library templates (e.g., generated from a single DNA sample) may be introduced into the flow cell 10E together. In this example, the different library templates 56 will seed at random unblocked primers, e.g., un-cleavable first primer 42, 42′ across the substrate 12.

The library templates 56 used in this example method may be prepared as described herein. The library templates 56 may also be introduced into the flow cell 10E and seeded as described in reference to FIG. 5A.

In one example of this method, the library template 56 is introduced and seeded, and then amplification of the seeded library template 56 is initiated using the first primer set 18, and then the second primer set 20 is introduced into the flow cell 10E after amplification takes place. In this example, the method moves from FIG. 8A to FIG. 8C, where FIG. 8C depicts the flow cell 10E after both processes have been performed. In another example of this method, the library template 56 is introduced and seeded, and then the second primer set 20 is introduced into the flow cell 10E, and then amplification of the library template 56 is initiated using the first primer set 18. In this example, the method moves from FIG. 8A to FIG. 8B to FIG. 8C. Library template 56 amplification and second primer set 20 introduction will now be described.

In the example where amplification takes place immediately after seeding, the polymerase used in amplification is introduced into the flow cell 10E with the library templates 56 and the amplification process may be performed as defined in reference to FIG. 5B. After the amplicons 58A, 58B are generated, the flow cell 10E may be washed as described herein. Following washing, the grafting mix is introduced into the flow cell 10E, and the primers 34, 34′ and 36, 36′ of the second primer set 20 react/interact with surface groups of the polymeric hydrogel 28″ to become attached thereto.

Alternatively, grafting of the second primer set 20 may take place before amplification. In this example, the library templates 56 are introduced into the flow cell 10E without the polymerase used for amplification. After seeding takes place, the flow cell 10E may be washed as described herein. Following washing, the grafting mix is introduced into the flow cell 10E, and the primers 34, 34′ and 36, 36′ of the second primer set 20 react/interact with surface groups of the polymeric hydrogel 28″ to become attached thereto. The grafting temperature may be about 60° C. After grafting takes place, the flow cell 10E may be washed again. Following the additional wash, the polymerase used in amplification is introduced into the flow cell 10E and the temperature may be adjusted to initiate amplification (e.g., 37° C.). It is to be understood that when grafting of the second primer set 20 takes place prior to library template amplification, the second primer set 20 does not participate because the amplified library templates 56 have already been seeded to the first primer set 18.

The amplification process generates the amplicons 58A, 58B, which are respectively attached to the un-cleavable primer 42 and the cleavable primer 40. The amplicons 58B attached to the cleavable primers 40 are then removed, as shown in FIG. 8D. The amplicons 58B are removed using a cleaving agent for the cleavage site 38′. The cleaving agent is introduced into the flow cell 10E and is allowed to incubate for a time sufficient to cleave the cleavage site 38′.

A washing process may then be performed to remove the cleaving agent and the cleaved amplicons 58B from the flow cell 10E. Because the amplicons 58A are attached to the un-cleavable primers 42, they will remain attached after the cleaving agent is removed from the flow cell 10E.

An extension reaction is performed along the first amplicon 58A to generate a first amplicon complement 58A′. This is also depicted in FIG. 8D. The extension reaction may be performed as described in reference to FIG. 5C.

The method continues with dehybridizing the first amplicon complement 58A′ from the first amplicon 58A (FIG. 8E); and initiating seeding of the first amplicon complement 58A′ to one of the primers, e.g., cleavable first primer 34, 34′, of the second primer set 20 (FIG. 8E). Dehybridization of the first amplicon complement 58A′ is performed as described in reference to FIG. 5D. At least some of the released first amplicon complements 58A′ will diffuse through the channel 16 toward the primer set, e.g., set 20, and will respectively seed to one of the primers, e.g., cleavable first primer 34, 34′, of the second primer set 20 (FIG. 8E). The first amplicon complements 58A′ will randomly seed on the substrate, e.g., 12″.

Amplification of the seeded first amplicon complements 58A′ may be performed in the same manner as the amplification of the initially seeded library template 56. This amplification process generates a third amplicon 58C attached to the cleavable first primer 34, 34′ and a fourth amplicon 58D attached to the un-cleavable second primer 36, 36′. The flow cell 10E after the second round of amplification is shown in FIG. 8F.

The method then includes cleaving the third amplicon 58C from the cleavable second primer 36, 36′; and initiating a sequencing operation of the first amplicon 58A and the fourth amplicon 58D.

In this example, the amplicons 58C attached to the cleavable first primer 34, 34′ are removed using a cleaving agent for the cleavage site 38. The cleaving agent that is introduced depends upon the cleavage site 38 that is to be cleaved. Any of the examples disclosed herein may be used. The cleaving agent is introduced into the flow cell 10E and is allowed to incubate for a time sufficient to cleave the cleavage site 38. The washing process may then be performed to remove the cleaving agent and the cleaved amplicons 58C.

As the amplicons 58D are attached to the un-cleavable primers 36, 36′, they will remain attached after the cleaving agent is removed from the flow cell 10E.

Due to the sequences and orthogonal cleavage sites 38, 38′ of the primer sets 20, 18, the first amplicon 58A and the fourth amplicon 58D are opposite strands (forward and reverse strands, or vice versa) of the initially introduced library templates 56. This enables simultaneous paired-end sequencing, which can be performed using a sequencing-by-synthesis method as described herein in reference to the FIG. 5 series.

During analysis of the sequencing data, the UMI or UDI may be used to bioinformatically link the amplicons 58A, 58D generated from the same library template 56. As an example, the method may include linking reads of the first amplicon 58A with reads of the fourth amplicon 58D by the unique molecular index (UMI) that is part of the library template 56 (from which the amplicons 58A, 58D are generated). As another example, the method may further include linking reads of the first amplicon 58A with reads of the fourth amplicon 58D by two indexes (part of a UDI) that are part of the library template 56 (from which the amplicons 58A, 58D are generated).

Flow Cells with Unblocked Primers

Another example of the method disclosed herein uses one flow cell with unblocked primers in each of the lanes, or two different flow cells with unblocked primers in each of the lanes. The unblocked primers are part of a set that includes two orthogonally cleavable primers that do not include the removable blocking mechanism 30 described herein.

FIG. 9 illustrates an example flow cell 10F with two lanes 14A, 14B, each of which contains primers 62A, 64A or 62B, 64B, which are unblocked. The primers 62A, 64A or 62B, 64B are similar to the primers 34, 34′ and 36, 36′ of the primer set 20 or to the primers 40, 40′ and 42, 42′ of the primer set 18 in that they have different sequences for respectively attaching the opposed adapters of the library template 56. However, each of the first and second primers 62A, 64A or 62B, 64B has a cleavage site 38, 38′, which is unlike the cleavable and un-cleavable primers of each of the primer sets 18, 20.

Examples of the primers 62A, 62B include the cleavable P5 primers set forth herein (see SEQ. ID. NOs. 8 and 9), and examples of the primers 64A, 64B include the cleavable P7 primers set forth herein (see SEQ. ID. NOs. 10-12). It is to be understood that the primers 62A and 62B are the same, and the primers 64A and 64B are the same, and thus the same primers are immobilized within each of the lanes 14A, 14B of the substrate 12.

In addition to the primers 62A, 62B, 64A, 64B, the flow cell 10F includes the substrate 12, the lanes 14A, 14B defined in the substrate 12, the polymeric hydrogel 28 positioned within each of the lanes 14A, 14B, and the lid 22 attached to the substrate 12. The substrate 12, the polymeric hydrogel 28, and the lid 22 may be any of the examples set forth herein. Any desired even number of lanes 14A, 14B may be defined in the substrate 12 using the techniques defined herein so that two lanes 14A, 14B make up a pair where forward and reverse strands of library templates 56 can be simultaneously sequenced. The lid 22 may be secured to the surface 24 of the substrate 12 using any technique described herein.

In this example flow cell 10F, a fluid line (not shown) may connect the two lanes 14A, 14B. The fluid line may be part of the flow cell 10F or a separate component of a sequencing instrument into which the flow cell 10F is placed. The fluid line may have a one-way valve that can be opened to allow fluid from the lane 14A to enter lane 14B. The fluid line may also include a bypass valve that can be opened to allow fluid to exit the lane 14A without entering into lane 14B. Alternatively, fluid from the lane 14A could be collected from the outlet and introduced into the inlet of the lane 14B.

Rather than a single flow cell 10F, FIG. 9 could alternatively represent two flow cells 10F, 10F2 that are used in the method described in reference to the FIGS. 10 series. The phantom line illustrates the division of the two flow cells 10F and 10F2, each of which includes the substrate 12 with the respective lane 14A or 14B defined therein, the polymeric hydrogel 28 within the lane 14A, 14B, the primers 62A, 62B, 64A, 64B immobilized to the polymeric hydrogel 28 within the lane 14A, 14B, the lid 22 attached to the substrate 12. The separate flow cells 10F, 10F2 make up a pair where forward and reverse strands of library templates 56 can be simultaneously sequenced.

Still further, the lanes 14A, 14B of the flow cell 10F or the separate flow cells 10F, 10F2 may include depressions as described herein, where the primers 62A, 64A and 62B, 64B are respectively located within separate depression. This/these patterned flow cell(s) may be used in the method(s) described in reference to the FIG. 10 series, where the designated reactions take place within the depressions.

While FIG. 9 illustrates the primers 62A, 62B, 64A, 64B, it is to be understood that this example flow cell 10F or 10F, 10F2 could include the primer sets (e.g., 18 in one lane 14A and 20 in lane 14B) as described herein, except that the cleavage sites of the cleavable primers 34, 40 would be the same.

In still another example, the flow cell(s) 10F or 10F and 10F2 could be modified for use with the RCA products or on-board amplification. In these examples, the capture primers 41 would be present in each of the lanes 14A, 14B or separate flow cells.

Methods for Using Flow Cells with Unblocked Primers

The flow cell 10F or flow cells 10F and 10F2 shown in FIG. 9 may be used in the method that is shown schematically in FIG. 10A through FIG. 10F. While the single flow cell 10F is described, it is to be understood that the method could be performed using the two separate flow cells 10F and 10F2. The method includes introducing a library template 56 into a first lane 14A of a flow cell 10F including first and second primers 62A, 64A with orthogonal cleaving chemistry, whereby the library template 56 seeds to one of the first and second primers 62A, 64A (FIG. 10A); initiating amplification of the library template 56 using the first and second primers 62A, 64A, thereby generating a first amplicon 58A attached to the first primer 62A and a second amplicon 58B attached to the second primer 64A (FIG. 10B); cleaving the second amplicon 58B from the second primer 64A, whereby the first amplicon 58A remains attached to the first primer 62A (FIG. 10C); performing an extension reaction along the first amplicon 58A to generate a first amplicon complement 58A′ (FIG. 10C); dehybridizing the first amplicon complement 58A′ from the first amplicon 58A (FIG. 10D); introducing the first amplicon complement 58A′ into a second lane 14B of the flow cell 10F or a second flow cell 10F2 that includes third and fourth primers 62B, 64B with orthogonal cleaving chemistry, whereby the first amplicon complement 58A′ seeds to one of the third and fourth primers 62B, 64B (FIG. 10D); initiating amplification of the first amplicon complement 58A′ using the third and fourth primers 62B, 64B, thereby generating a third amplicon 58C attached to the third primer 62B and a fourth amplicon 58D attached to the fourth primer 64B (FIG. 10E); cleaving the third amplicon 58C from the third primer 62B (FIG. 10F); and simultaneously initiating sequencing of the first amplicon 58A in the first lane 14A of the flow cell 10F and of the fourth amplicon 58D in the second lane 14B of the flow cell 10F or in the second flow cell 10F2.

In FIG. 10A, the library template 56 is introduced into the lane 14A of the flow cell 10F. While a single library template 56 is shown, it is to be understood that a plurality of different library templates (e.g., generated from a single DNA sample) may be introduced into the flow cell 10F together. In this example, the different library templates 56 will seed at random primers 62A, 64A across the substrate 12.

The library templates 56 used in this example method may be prepared as described herein. The library templates 56 may also be introduced into the lane 14A of the flow cell 10F and seeded as described in reference to FIG. 5A.

In this example, amplification takes place immediately after seeding. The polymerase used in amplification is introduced into the flow cell 10F with the library templates 56 and the amplification process may be performed as defined in reference to FIG. 5B. After the amplicons 58A, 58B are generated (shown in FIG. 10B), the flow cell lane 14A may be washed as described herein.

The amplification process generates the amplicons 58A, 58B, which are respectively attached to the first and second primers 62A, 64A. The amplicons 58B attached to the second primers 64A are then removed, as shown in FIG. 10C. The amplicons 58B are removed using a cleaving agent for the cleavage site 38′. The cleaving agent is introduced into the lane 14A and is allowed to incubate for a time sufficient to cleave the cleavage site 38′.

A washing process may then be performed to remove the cleaving agent and the cleaved amplicons 58B from the lane 14A. Because the amplicons 58A are attached to the first primers 62A and the cleavage site 38 of these primers 62A is orthogonal to the cleavage site 38′ of the primers 64A, they will remain attached after the cleaving agent is removed from the lane 14A. The removed cleaving agent and amplicons 58B may be directed to a waste container (as opposed to into the lane 14B or another flow cell 10F2).

An extension reaction is performed along the first amplicon 58A to generate a first amplicon complement 58A′. This is also depicted in FIG. 10C. The extension reaction may be performed as described in reference to FIG. 5C.

The method continues with dehybridizing the first amplicon complement 58A′ from the first amplicon 58A (FIG. 10D); and initiating seeding of the first amplicon complement 58A′ to one of the primers 62B, 64B in the second lane 14B or second flow cell 10F2 (FIG. 10D). Dehybridization of the first amplicon complement 58A′ is performed as described in reference to FIG. 5D. At least some of the released first amplicon complements 58A′ will be collected from the lane 14A through its outlet and introduced into the lane 14B through its inlet. Alternatively, the fluid line between the lanes 14A, 14B may be opened and the released first amplicon complements 58A′ will be directed from the lane 14A into the lane 14B. In the lane 14B, the amplicon complements 58A′ will respectively and randomly seed to one of the primers 62B, 64B (FIG. 10D).

Amplification of the seeded first amplicon complements 58A′ may be performed in the same manner as the amplification of the initially seeded library template 56. This amplification process generates a third amplicon 58C attached to the primer 62B and a fourth amplicon 58D attached to the primer 64B. The flow cell 10F or flow cells 10F and 10F2 after the second round of amplification is shown in FIG. 10E.

The method then includes cleaving the third amplicon 58C from the primer 62B; and initiating a sequencing operation of the first amplicon 58A and the fourth amplicon 58D.

In this example, the amplicons 58C attached to the third primer 62B are removed using a cleaving agent for the cleavage site 38. The cleaving agent that is introduced depends upon the cleavage site 38 that is to be cleaved. Any of the examples disclosed herein may be used. The cleaving agent is introduced into the flow cell 10E and is allowed to incubate for a time sufficient to cleave the cleavage site 38. The washing process may then be performed to remove the cleaving agent and the cleaved amplicons 58C.

As the amplicons 58D are attached to the primers 64B with the orthogonal cleavage site, they will remain attached after the cleaving agent is removed from the lane 14B of flow cell 10F or from the flow cell 10F2.

Due to the sequences and orthogonal cleavage sites 38, 38′ of the primers 62A, 64B and 62B, 64B, the first amplicon 58A and the fourth amplicon 58D are opposite strands (forward and reverse strands, or vice versa) of the initially introduced library templates 56. This enables simultaneous paired-end sequencing, which can be performed using a sequencing-by-synthesis method as described herein in reference to the FIG. 5 series. In this example, sequencing-by-synthesis is performed in each of the lanes 14A, 14B.

During analysis of the sequencing data, the UMI or UDI may be used to bioinformatically link the amplicons 58A, 58D generated from the same library template 56. As an example, the method may include linking reads of the first amplicon 58A with reads of the fourth amplicon 58D by the unique molecular index (UMI) that is part of the library template 56 (from which the amplicons 58A, 58D are generated). As another example, the method may further include linking reads of the first amplicon 58A with reads of the fourth amplicon 58D by two indexes (part of a UDI) that are part of the library template 56 (from which the amplicons 58A, 58D are generated).

The method set forth in the FIG. 5 series may be performed as described in the FIG. 10 series, where the first primer set 18 is in one lane 14A or flow cell and the second primer set 20 is in a second lane 14B or flow cell. As described in reference to FIG. 9, however, the cleavable primers 34, 40 include the same type of cleavage site.

Alternatively, any of the method set forth using RCA products or on-board RCA amplification may be performed in the flow cell of FIG. 9, where the RCA products are seeded or generated in the lane 14A and the copies are transferred to lane 14B for seeding, amplification, and sequencing.

### Complementary Metal Oxide Flow Cells

Another example of the flow cell architecture is shown in FIG. 11. This example depicts an example of the flow cell 10C that is integrated with a complementary metal oxide semiconductor (CMOS) chip 70.

In this example flow cell 10C, the substrate 12 is positioned over the CMOS chip 70. The substrate 12 includes a plurality of depressions 66 defined therein, which are separated by interstitial regions 68. The polymeric hydrogel 28A, 28B is positioned on a bottom surface in each of the plurality of depressions 66, and the primer sets 18, 20 (one of which is blocked with the removable blocking mechanism 30) are respectively attached to the hydrogels 28A, 28B.

In this example, the substrate 12 may be a passivation layer. With the passivation layer as the substrate 12, the substrate 12 may provide one level of corrosion protection for an embedded metal layer 78 of the CMOS chip 70 that is closest in proximity to the substrate 12. In this example, the substrate 12 may include a passivating material that is transparent to the light emissions (e.g., visible light) resulting from reactions at the primer sets 18, 20, and that is at least initially resistant to the fluidic environment and moisture that may be introduced into or present in the flow channel 16. An “at least initially resistant material” acts as an etch barrier to high pH reagents (e.g., pH ranging from 8 to 14) and as a moisture barrier. Examples of suitable passivation materials for the substrate 12 include silicon nitride (Si₃N₄), silicon oxide (SiO₂), tantalum pentoxide (TaO₅), hafnium oxide (HfO₂), boron doped p+ silicon, or the like. The thickness of the substrate 12 ranges from about 100 nm to about 500 nm.

As mentioned, at least a portion of the substrate 12 (i.e., the passivation layer) is in contact with the first embedded metal layer 78 of the CMOS chip 70 and also with an input region 80 of the optical waveguide 76. The contact between the substrate 12 and the first embedded metal layer 78 may be direct contact or may be indirect contact through a shield layer 82.

In the illustrated example, the substrate 12 may be affixed directly to, and thus be in physical contact with, the CMOS chip 70 through one or more securing mechanisms (e.g., adhesive, bond, fasteners, and the like). It is to be understood that the substrate 12 may be removably coupled to the CMOS chip 70.

The CMOS chip 70 includes a plurality of stacked layers 72 including, for example, silicon layer(s), dielectric layer(s), metal-dielectric layer(s), metal layer(s), etc.). The stacked layers 72 make up the device circuitry, which includes detection circuitry.

The CMOS chip 70 includes optical components, such as optical sensor(s) 74 and optical waveguide(s) 76. The optical components are arranged such that each optical sensor 74 at least substantially aligns with, and thus is operatively associated with, a single optical waveguide 76 and a single reaction site (i.e., each location where the hydrogels 28A, 28B and primer sets 18, 20 are located) of the flow cell 10C. However, in other examples, a single optical sensor 74 may receive photons through more than one optical waveguide 76 and/or from more than one reaction site. In these other examples, the single optical sensor 74 is operatively associated with more than one optical waveguide 76 and/or more than one reaction site.

As used herein, a single optical sensor 74 may be a light sensor that includes one pixel or more than one pixel. As an example, each optical sensor 74 may have a detection area that is less than about 50 μm². As another example, the detection area may be less than about 10 μm². As still another example, the detection area may be less than about 2 μm². In the latter example, the optical sensor 74 may constitute a single pixel. An average read noise of each pixel the optical sensor 74 may be, for example, less than about 150 electrons. In other examples, the read noise may be less than about 5 electrons. The resolution of the optical sensor(s) 98 may be greater than about 0.5 megapixels (Mpixels). In other examples, the resolution may be greater than about 5 Mpixels, or greater than about 10 Mpixels.

Also as used herein, a single optical waveguide 76 may be a light guide including a cured filter material that i) filters the excitation light 84 (propagating from an exterior of the flow cell 10C into the flow channel 16), and ii) permits the light emissions (not shown, resulting from reactions at the reaction site(s)) to propagate therethrough toward corresponding optical sensor(s) 74. In an example, the optical waveguide 76 may be, for example, an organic absorption filter. As a specific example, the organic absorption filter may filter excitation light 84 of about 532 nm wavelength and permit light emissions of about 570 nm or more wavelengths. The optical waveguide 76 may be formed by first forming a guide cavity in a dielectric layer 86, and then filling the guide cavity with a suitable filter material.

The optical waveguide 76 may be configured relative to the dielectric material 86 in order to form a light-guiding structure. For example, the optical waveguide 76 may have a refractive index of about 2.0 so that the light emissions are substantially reflected at an interface between the optical waveguide 76 and the surrounding dielectric material 86. In certain examples, the optical waveguide 76 is selected such that the optical density (OD) or absorbance of the excitation light 84 is at least about 4 OD. More specifically, the filter material may be selected and the optical waveguide 76 may be dimensioned to achieve at least 4 OD. In other examples, the optical waveguide 76 may be configured to achieve at least about 5 OD or at least about 6 OD.

The flow cell 10C also includes the lid 22 that is operatively connected to the substrate 12 to partially define the flow channel 16 between the substrate 12 (and the reaction site(s) therein or thereon) and the lid 22. The lid 22 may be any of the example materials set forth herein that are transparent to the excitation light 84 that is directed toward the reaction site(s).

The lid 22 may include inlet and outlet ports 92, 94 that are configured to fluidically engage other ports (not shown) for directing fluid(s) into the flow channel 16 (e.g., from a reagent cartridge or other fluid storage system component) and out of the flow channel 16 (e.g., to a waste removal system).

The flow channel 16 may be sized and shaped to direct a fluid along the reaction site(s). The height of the flow channel 16 and other dimensions of the flow channel 16 may be configured to maintain a substantially even flow of the fluid along the reaction site(s). The dimensions of the flow channel 16 may also be configured to control bubble formation. In an example, the height of the flow channel 50 may range from about 50 μm to about 400 μm. In another example, the height of the flow channel 16 may range from about 80 μm to about 200 μm. It is to be understood that the height of the flow channel 16 may vary, and may be the greatest when the reaction site is located in a reaction chamber (e.g., depression 66) that is defined in the surface of the substrate 12. In these examples, the depression 66 increases the height of the flow channel 16 at this particular area.

Each reaction site is a localized region in the substrate 12 where the primer sets 18, 20 (or capture primers 41 or primers 62A, 64A) are positioned and where designated reactions involving these components may occur.

In an example, the reaction site is at least substantially aligned with the input region 80 of a single optical waveguide 76. As such, light emissions at the reaction site may be directed into the input region 80, through the waveguide 76, and to an associated optical sensor 74. In other examples, one reaction site may be aligned with several input regions 80 of several optical waveguides 76. In still other examples, several reaction sites may be aligned with one input region 80 of one optical waveguide 76.

The embedded metal layer 78 may be any suitable CMOS metal, such as aluminum (Al), aluminum chloride (AICu), tungsten (W), nickel (Ni), or copper (Cu). The embedded metal layer 78 is a functioning part of the CMOS AVdd line, and through the stacked layers 72, is also electrically connected to the optical sensor 74. Thus, the embedded metal layer 78 participates in the detection/sensing operation.

It is to be understood that the other optical sensors 74 and associated components may be configured in an identical or similar manner. It is also to be understood, however, that the CMOS chip 70 may not be manufactured identically or uniformly throughout. Instead, one or more optical sensor 74 and/or associated components may be manufactured differently or have different relationships with respect to one another

The stacked layers 72 may include interconnected conductive elements (e.g., conductors, traces, vias, interconnects, etc.) that can conduct electrical current. The circuitry may be configured for selectively transmitting data signals that are based on detected photons. The circuitry may also be configured for signal amplification, digitization, storage, and/or processing. The circuitry may collect and analyze the detected light emissions and generate data signals for communicating detection data to a bioassay system. The circuitry may also perform additional analog and/or digital signal processing in the CMOS chip 70.

The CMOS chip 70 may be manufactured using integrated circuit manufacturing processes. The CMOS chip 70 may include multiple layers, such as a sensor base/layer (e.g., a silicon layer or wafer or dielectric layer 86). The sensor base may include the optical sensor 74. When the CMOS chip 70 is fully formed, the optical sensor 74 may be electrically coupled to the rest of the circuitry in the stacked layers 72 through gate(s), transistor(s), etc.

As used in reference to FIG. 11, the term “layer” is not limited to a single continuous body of material unless otherwise noted. For example, the sensor base/layer may include multiple sub-layers that are different materials and/or may include coatings, adhesives, and the like. Furthermore, one or more of the layers (or sub-layers) may be modified (e.g., etched, deposited with material, etc.) to provide the features described herein.

The stacked layers 72 also include a plurality of metal-dielectric layers. Each of these layers includes metallic elements (e.g., M1-M5, which may be, for example, W (tungsten), Cu (copper), Al (aluminum), or any other suitable CMOS conductive material) and dielectric material 86 (e.g., SiO₂). Various metallic elements M1-M5 and dielectric materials 86 may be used, such as those suitable for integrated circuit manufacturing.

In the example shown in FIG. 11, each of the plurality of metal-dielectric layers L1-L6 includes both metallic elements M1, M2, M3, M4, M5 and dielectric material 86. In each of the layers L1-L6, the metallic elements M1, M2, M3, M4, M5 are interconnected and are embedded within dielectric material 86. In some of the metal-dielectric layers L1-L6, additional metallic elements may also be included. Some of these additional metallic elements may be used to address individual pixels through a row and column selector. The voltages at these elements may vary and switch between about-1.4 V and about 4.4 V depending upon which pixel the device is reading out.

The configuration of the metallic elements M1, M2, M3, M4, M5 and dielectric layer 86 in FIG. 11 is illustrative of the circuitry, and it is to be understood that other examples may include fewer or additional layers and/or may have different configurations of the metallic elements M1-M5.

In the example shown in FIG. 11, the shield layer 82 is in contact with at least a portion of the substrate 12. The shield layer 82 has an aperture at least partially adjacent to the input region 80 of the optical waveguide 76. This aperture enables the reaction site (and at least some of the light emissions therefrom) to be optically connected to the waveguide 76. It is to be understood that the shield layer 82 may have an aperture at least partially adjacent to the input region 80 of each optical waveguide 76. The shield layer 82 may extend continuously between adjacent apertures.

The shield layer 82 may include any material that can block, reflect, and/or significantly attenuate the light signals that are propagating through the flow channel 16. The light signals may be the excitation light 84 and/or the light emissions from the reaction site(s). As an example, the shield layer 82 may be tungsten (W).

It is to be understood that the flow cell 10C (or any of the other flow cells described herein integrated with the CMOS chip 70) may also be used for optical detection.

### Additional Notes

Any of the examples including the regions A, B or the separate lanes 14A, 14B or flow cells 10F, 10F2 can be performed with a temporary lid, which is removed after amplicon generation and prior to sequencing. The temporary lid may be any of the examples set forth herein and may be removed using any suitable de-bonding process.

In any of the examples including the second substrate 12′, 12″, after the amplicons are generated on the desired substrate or region and prior to sequencing, the flow cell 10A, 10B, 10E, 10G can be de-bonded. Each of the open substrates 12, 12′, 12″ can then be sequenced.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with0 the example is included in at least one example described herein, and may or may not be present in other examples. In addition, it is to be understood that the described elements for any example may be combined in any suitable manner in the various examples unless the context clearly dictates otherwise.

While several examples have been described in detail, it is to be understood that the disclosed examples may be modified. Therefore, the foregoing description is to be considered non-limiting.

1. A flow cell, comprising:
a first substrate;
a second substrate attached to the first substrate;
a flow channel defined between the first substrate and the second substrate;
a first primer set attached to the first substrate, the first primer set including an un-cleavable first primer and a cleavable second primer;
a second primer set attached to the second substrate, the second primer set including a cleavable first primer and an un-cleavable second primer; and
a removable blocking mechanism passivating i) the first primer set or the second primer set or ii) the cleavable first primer and the cleavable second primer.

2. The flow cell as defined in claim 1, wherein:
the removable blocking mechanism is passivating i) the first primer set or the second primer set;
the removable blocking mechanism includes a plurality of linearizable blocking primers grafted to the first substrate or to the second substrate; and
the plurality of linearizable blocking primers is hybridized to the un-cleavable first primer and the cleavable second primer of the first primer set or to the cleavable first primer and the un-cleavable second primer of the second primer set.

3. The flow cell as defined in claim 1, wherein:
the removable blocking mechanism is passivating i) the first primer set or the second primer set;
the removable blocking mechanism includes a plurality of blocking primers; and
the plurality of blocking primers is respectively to the un-cleavable first primer and the cleavable second primer of the first primer set or to the cleavable first primer and the un-cleavable second primer of the second primer set.

4. The flow cell as defined in claim 3, wherein:
the plurality of blocking primers is passivating the first primer set; and
the plurality of blocking primers includes a first blocking primer that is complementary to the un-cleavable first primer and a second blocking primer that is complementary to the cleavable second primer.

5. The flow cell as defined in claim 3, wherein:
the plurality of blocking primers is passivating the second primer set; and
the plurality of blocking primers includes a first blocking primer that is complementary to the cleavable first primer and a second blocking primer that is complementary to the un-cleavable second primer.

6. The flow cell as defined in claim 1, wherein:
the removable blocking mechanism is passivating the first primer set;
the first primer set includes a first linearizable hairpin primer and a second linearizable hairpin primer;
the first linearizable hairpin primer includes:
the un-cleavable first primer; and
a first hairpin portion attached at a 3′ end of the un-cleavable first primer, the first hairpin portion including:
a cleavage site that is orthogonal to a cleavage site of the cleavable second primer; and
a first hairpin formation sequence that is hybridized to at least a portion of the un-cleavable first primer;
the second linearizable hairpin primer includes:
the cleavable second primer; and
a second hairpin portion attached at a 3′ end of the cleavable second primer, the second hairpin portion including:
the cleavage site that is orthogonal to the cleavage site of the cleavable second primer; and
a second hairpin formation sequence that is hybridized to at least a portion of the cleavable second primer; and
the removable blocking mechanism includes both the first hairpin formation sequence and the second hairpin formation sequence.

7. The flow cell as defined in claim 1, wherein:
the removable blocking mechanism is passivating the second primer set;
the second primer set includes a first linearizable hairpin primer and a second linearizable hairpin primer;
the first linearizable hairpin primer includes:
the cleavable first primer; and
a first hairpin portion attached at a 3′ end of the cleavable first primer, the first hairpin portion including:
a cleavage site that is orthogonal to a cleavage site of the cleavable first primer; and
a first hairpin formation sequence that is hybridized to at least a portion of the cleavable first primer;
the second linearizable hairpin primer includes:
the un-cleavable second primer; and
a second hairpin portion attached at a 3′ end of the un-cleavable second primer, the second hairpin portion including:
the cleavage site that is orthogonal to the cleavage site of the cleavable first primer; and
a second hairpin formation sequence that is hybridized to at least a portion of the un-cleavable second primer; and
the removable blocking mechanism includes both the first hairpin formation sequence and the second hairpin formation sequence.

8. The flow cell as defined in claim 1, wherein:
the removable blocking mechanism is passivating ii) the cleavable first primer and the cleavable second primer; and
the removable blocking mechanism is a terminal phosphate group at a 3′ end of each of the cleavable first primer and the cleavable second primer.

9. The flow cell as defined in claim 1, wherein:
the removable blocking mechanism is passivating ii) the cleavable first primer and the cleavable second primer;
the cleavable first primer is a part of a first linearizable hairpin primer including a first hairpin portion attached at a 3′ end of the cleavable first primer, the first hairpin portion including:
a cleavage site that is orthogonal to a cleavage site of each of the cleavable first primer and the cleavable second primer; and
a first hairpin formation sequence that is hybridized to at least a portion of the cleavable first primer;
the cleavable second primer is a part of a second linearizable hairpin primer including a second hairpin portion attached at a 3′ end of the cleavable second primer, the second hairpin portion including:
the cleavage site that is orthogonal to a cleavage site of each of the cleavable first primer and the cleavable second primer; and
a second hairpin formation sequence that is hybridized to at least a portion of the cleavable second primer;
the removable blocking mechanism includes both the first hairpin formation sequence and the second hairpin formation sequence.

10. The flow cell as defined in claim 1, wherein:
the removable blocking mechanism is passivating i) the first primer set or the second primer set; and
the removable blocking mechanism is a removable hydrophobic barrier layer overlying the first primer set or the second primer set.

11. The flow cell as defined in claim 1, wherein:
the flow cell includes a plurality of the first primer set and a plurality of the second primer set;
some of the cleavable second primers of the plurality of the first primer set include a first cleavage site and some other of the cleavable second primers of the plurality of the first primer set include a second cleavage site that is orthogonal to the first cleavage site; and
the cleavable first primers of the plurality of the second primer set include the second cleavage site.

12.-23. (canceled)

24. A method, comprising:
introducing a library template into a flow cell including spatially separated first and second primer sets, wherein:
the first primer set includes an un-cleavable first primer and a cleavable second primer;
the second primer set includes a cleavable first primer and an un-cleavable second primer; and
the cleavable first primer and the un-cleavable second primer of the second primer set are passivated by a removable blocking mechanism,
whereby the library template seeds to one of the primers of the first primer set;
initiating amplification of the library template using the first primer set while the cleavable first primer and the un-cleavable second primer of the second primer set are passivated, thereby generating a first amplicon attached to the un-cleavable first primer and a second amplicon attached to the cleavable second primer of the first primer set;
cleaving the second amplicon from the cleavable second primer;
performing an extension reaction along the first amplicon to generate a first amplicon complement;
removing the removable blocking mechanism from the cleavable first primer and the un-cleavable second primer of the second primer set;
dehybridizing the first amplicon complement from the first amplicon; and
initiating seeding of the first amplicon complement to one of the primers of the second primer set.

25. The method as defined in claim 24, wherein:
the removable blocking mechanism includes a plurality of linearizable blocking primers grafted in a region with the second primer set;
during the amplification, one of the plurality of linearizable blocking primers is respectively hybridized to the cleavable first primer and the un-cleavable second primer of the second primer set; and
removing the removable blocking mechanism involves introducing a cleaving agent to the flow cell at a temperature that dehybridizes each of the plurality of linearizable blocking primers from the cleavable first primer and the un-cleavable second primer of the second primer set, whereby the cleaving agent cleaves a cleavage site of each of the plurality of linearizable blocking primers.

26. The method as defined in claim 25, wherein the cleaving agent also cleaves the second amplicon from the cleavable second primer.

27. The method as defined in claim 24, wherein:
the removable blocking mechanism includes a plurality of blocking primers;
during the amplification, the plurality of blocking primers is hybridized to the cleavable first primer and the un-cleavable second primer of the second primer set; and
removing the removable blocking mechanism involves:
dehybridizing the plurality of blocking primers from the cleavable first primer and the un-cleavable second primer of the second primer set; and
washing the plurality of blocking primers from the flow cell before cleaving the second amplicon from the cleavable second primer.

28. The method as defined in claim 27, wherein the plurality of blocking primers includes a first blocking primer that is complementary to the cleavable first primer and a second blocking primer that is complementary to the un-cleavable second primer.

29. The method as defined in claim 24, wherein:
the removable blocking mechanism is respectively integrated into a first linearizable hairpin primer with the cleavable first primer and a second linearizable hairpin primer with the un-cleavable second primer;
during the amplification, a first hairpin formation sequence of the first linearizable hairpin primer is hybridized to at least a portion of the cleavable first primer and a second hairpin formation sequence of the second linearizable hairpin primer is hybridized to at least a portion of the un-cleavable second primer; and
removing the removable blocking mechanism involves introducing a cleaving agent of the first linearizable hairpin primer and the second linearizable hairpin primer.

30. The method as defined in claim 29, wherein the cleaving agent of the first linearizable hairpin primer and the second linearizable hairpin primer also cleaves the second amplicon.

31. The method as defined in claim 24, wherein:
the removable blocking mechanism is a hydrophobic barrier layer overlying the second primer set; and
removing the removable blocking mechanism involves washing the hydrophobic barrier layer from the flow cell before cleaving the second amplicon from the cleavable second primer.

32. The method as defined in claim 24, further comprising:
initiating amplification of the first amplicon complement using the second primer set, thereby generating a third amplicon attached to the cleavable first primer and a fourth amplicon attached to the un-cleavable second primer;
cleaving the third amplicon from the cleavable second primer; and
initiating a sequencing operation of the first amplicon and the fourth amplicon.

33. The method as defined in claim 32, further comprising one of:
linking reads of the first amplicon with reads of the fourth amplicon by a unique molecular index that is part of the library template; or
linking reads of the first amplicon with reads of the fourth amplicon by two indexes that are part of the library template.

34. (canceled)

35. The method as defined in claim 24, wherein:
the flow cell includes a plurality of the first primer set and a plurality of the second primer set; and
the method further comprises initially grafting the plurality of the first primer set such that some of the cleavable second primers of the plurality of the first primer set include a first cleavage site and some other of the cleavable second primers of the plurality of the first primer set include a second cleavage site that is orthogonal to the first cleavage site.

36. The method as defined in claim 24, wherein:
a plurality of the first amplicon complement is generated; and
the method further comprises controlling a concentration of the plurality of the first amplicon complement by titrating a concentration of an extension primer used during the extension reaction.

37. The method as defined in claim 24, wherein after the dehybridization of the first amplicon complement, the method further comprises addressing an electrode of the flow cell that is spatially co-located with the second primer set, thereby electrophoretically attracting the first amplicon complement.

38. A method, comprising:
introducing complementary library template strands into a flow cell including spatially separated first and second primer sets, wherein:
the first primer set includes an un-cleavable first primer and a cleavable second primer;
the second primer set includes a cleavable first primer and an un-cleavable second primer; and
each of the cleavable first primer and the cleavable second primer is block with a removable blocking mechanism;
whereby the complementary library template strands respectively seed to the un-cleavable first primer and the un-cleavable second primer;
performing a strand extension reaction along the seeded complementary library template strands, thereby generating a first amplicon attached to the un-cleavable first primer and a second amplicon attached to the un-cleavable second primer;
dehybridizing the complementary library template strands from each of the first amplicon and the second amplicon;
removing the removable blocking mechanism from each of the cleavable first primer and from the cleavable second primer; and
initiating amplification of the first amplicon using the first primer set and the second amplicon using the second primer set.

39. The method as defined in claim 38, wherein:
the removable blocking mechanism is a terminal phosphate group at a 3′ end of each of the cleavable first primer and the cleavable second primer; and
removing the removable blocking mechanism involves dephosphorylating the terminal phosphate groups.

40. The method as defined in claim 38, further comprising generating the complementary library template strands by exposing an original library template strand to a polymerase chain reaction.

41. The method as defined in claim 38, wherein:
the removable blocking mechanism is respectively integrated into a first linearizable hairpin primer with the cleavable first primer and a second linearizable hairpin primer with the cleavable second primer;
during the strand extension reaction, a first hairpin formation sequence of the first linearizable hairpin primer is hybridized to at least a portion of the cleavable first primer and a second hairpin formation sequence of the second linearizable hairpin primer is hybridized to at least a portion of the cleavable second primer; and
removing the removable blocking mechanism involves introducing a cleaving agent of the first linearizable hairpin primer and the second linearizable hairpin primer.

42.-49. (canceled)
